SEQUENCE LISTING (200 sequences)

SEQ ID NO: 1 - protein, 20 aa, Artificial Sequence
  Fc region binding polypeptide
ETQRCTWHMGELVWCEREHN

SEQ ID NO: 2 - protein, 20 aa, Artificial Sequence
  Fc region binding polypeptide
KEASCSYWLGELVWCVAGVE

SEQ ID NO: 3 - protein, 6 aa, Artificial Sequence
  motif common to known Fc region binders
GELVWC

SEQ ID NO: 4-7 - skipped (4 entries)

SEQ ID NO: 8 - protein, 12 aa, Artificial Sequence
  variegation template for phage display library
XXXCXXXXCXXX

SEQ ID NO: 9 - protein, 13 aa, Artificial Sequence
  variegation template for phage display library
XXXCXXXXXCXXX

SEQ ID NO: 10 - protein, 14 aa, Artificial Sequence
  variegation template for phage display library
XXXCXXXXXXCXXX

SEQ ID NO: 11 - protein, 15 aa, Artificial Sequence
  variegation template for phage display library
XXXCXXXXXXXCXXX

SEQ ID NO: 12 - protein, 16 aa, Artificial Sequence
  variegation template for phage display library
XXXCXXXXXXXXCXXX

SEQ ID NO: 13 - protein, 18 aa, Artificial Sequence
  variegation template for phage display library
XXXCXXXXXXXXXXCXXX

SEQ ID NO: 14 - protein, 18 aa, Artificial Sequence
  Fc region binding polypeptide
RRACSRDWSGALVWCAGH

SEQ ID NO: 15 - protein, 18 aa, Artificial Sequence
  Fc region binding polypeptide
WGECTVTSYGELIWCGGL

SEQ ID NO: 16 - protein, 18 aa, Artificial Sequence
  Fc region binding polypeptide
SSACAFDPMGAVIWCTYD

SEQ ID NO: 17 - protein, 18 aa, Artificial Sequence
  Fc region binding polypeptide
LLECAYNTSGELIWCNGS

SEQ ID NO: 18 - protein, 18 aa, Artificial Sequence
  Fc region binding polypeptide
PDDCSIHFSGELIWCEPL

SEQ ID NO: 19 - protein, 18 aa, Artificial Sequence
  Fc region binding polypeptide
LGECTVTSYGELIWCGGL

SEQ ID NO: 20 - protein, 18 aa, Artificial Sequence
  Fc region binding polypeptide
WGECTVTSYGELIWCGGH

SEQ ID NO: 21 - protein, 18 aa, Artificial Sequence
  Fc region binding polypeptide
DHMCVYTTWGELIWCDDH

SEQ ID NO: 22 - protein, 18 aa, Artificial Sequence
  Fc region binding polypeptide
WGECTVTSYGELIWCGGL

SEQ ID NO: 23 - protein, 18 aa, Artificial Sequence
  Fc region binding polypeptide
CRACSRDWPGALVWCAGH

SEQ ID NO: 24 - protein, 18 aa, Artificial Sequence
  Fc region binding polypeptide
RRACSRDWSGALVWCAGH

SEQ ID NO: 25 - protein, 18 aa, Artificial Sequence
  Fc region binding polypeptide
LHACAFDPMGAVIWCTYD

SEQ ID NO: 26 - protein, 18 aa, Artificial Sequence
  Fc region binding polypeptide
DHMCVYTTWGELMWCDNH

SEQ ID NO: 27 - protein, 18 aa, Artificial Sequence
  Fc region binding polypeptide
PPTCTWDWQGILVWCSGH

SEQ ID NO: 28 - protein, 18 aa, Artificial Sequence
  Fc region binding polypeptide
SNKCSNTWDGSLIWCSAN

SEQ ID NO: 29 - protein, 18 aa, Artificial Sequence
  Fc region binding polypeptide
FPECTFDMEGFLIWCSSF

SEQ ID NO: 30 - protein, 18 aa, Artificial Sequence
  Fc region binding polypeptide
HDLCAQAPFGDATWCDLR

SEQ ID NO: 31 - protein, 18 aa, Artificial Sequence
  Fc region binding polypeptide
PNHCSYNLKSELIWCQDL

SEQ ID NO: 32 - protein, 18 aa, Artificial Sequence
  Fc region binding polypeptide
PLDCARDIHNSLIWCSLG

SEQ ID NO: 33 - protein, 18 aa, Artificial Sequence
  Fc region binding polypeptide
GSECSWTSLNELIWCAHW

SEQ ID NO: 34 - protein, 18 aa, Artificial Sequence
  Fc region binding polypeptide
WPDCSFTVQRDLIWCEAL

SEQ ID NO: 35 - protein, 18 aa, Artificial Sequence
  Fc region binding polypeptide
SHSCAYDYAHMLVWCTHF

SEQ ID NO: 36 - protein, 18 aa, Artificial Sequence
  Fc region binding polypeptide
DHMCVYTTWGELIWCDNH

SEQ ID NO: 37 - protein, 18 aa, Artificial Sequence
  Fc region binding polypeptide
RPNCTFAASGELIWCMHY

SEQ ID NO: 38 - protein, 18 aa, Artificial Sequence
  Fc region binding polypeptide
WWGCQFDWRGELVWCPYL

SEQ ID NO: 39 - protein, 18 aa, Artificial Sequence
  Fc region binding polypeptide
GGVCSYSGMGEIVWCRWF

SEQ ID NO: 40 - protein, 18 aa, Artificial Sequence
  Fc region binding polypeptide
ALMCSHDMWGSLIWCKHF

SEQ ID NO: 41 - protein, 18 aa, Artificial Sequence
  Fc region binding polypeptide
WWNCHNGWTWTGGWCWWF

SEQ ID NO: 42 - protein, 18 aa, Artificial Sequence
  Fc region binding polypeptide
YHVCARDSWDQLIWCEAF

SEQ ID NO: 43 - protein, 15 aa, Artificial Sequence
  Fc region binding polypeptide
NYWCNFWQLPTCDNL

SEQ ID NO: 44 - protein, 16 aa, Artificial Sequence
  Fc region binding polypeptide
YWYCKWFSESASCSSR

SEQ ID NO: 45 - protein, 16 aa, Artificial Sequence
  Fc region binding polypeptide
YWYCKWFEDKHPCDSS

SEQ ID NO: 46 - protein, 16 aa, Artificial Sequence
  Fc region binding polypeptide
YWYCSWFPDRPDCPLY

SEQ ID NO: 47 - protein, 16 aa, Artificial Sequence
  Fc region binding polypeptide
NYWCNVWLLGDVCRSH

SEQ ID NO: 48 - protein, 18 aa, Artificial Sequence
  Fc region binding polypeptide
LYWCHVWFGQHAWQCKYP

SEQ ID NO: 49 - protein, 14 aa, Artificial Sequence
  Fc region binding polypeptide
YWKCKWMPWMCGFD

SEQ ID NO: 50 - protein, 18 aa, Artificial Sequence
  Fc region binding polypeptide
DDHCYWFREWFNSECPHG

SEQ ID NO: 51 - protein, 15 aa, Artificial Sequence
  Fc region binding polypeptide
NYWCNIWGLHGCNSH

SEQ ID NO: 52 - protein, 16 aa, Artificial Sequence
  Fc region binding polypeptide
YWFCQWFSQNHTCFRD

SEQ ID NO: 53 - protein, 16 aa, Artificial Sequence
  Fc region binding polypeptide
HYWCDIWFGAPACQFR

SEQ ID NO: 54 - protein, 17 aa, Artificial Sequence
  Fc region binding polypeptide
SGDCGFWPRIWGLCMDN

SEQ ID NO: 55 - protein, 16 aa, Artificial Sequence
  Fc region binding polypeptide
FWYCKWFYEDAQCSHD

SEQ ID NO: 56 - protein, 13 aa, Artificial Sequence
  Fc region binding polypeptide
YYWCNYWGLCPDQ

SEQ ID NO: 57 - protein, 13 aa, Artificial Sequence
  Fc region binding polypeptide
SYWCKIWDVCPQS

SEQ ID NO: 58 - protein, 13 aa, Artificial Sequence
  Fc region binding polypeptide
KYWCNLWGVCPAN

SEQ ID NO: 59 - protein, 13 aa, Artificial Sequence
  Fc region binding polypeptide
QYWCYQWGLCGAN

SEQ ID NO: 60 - protein, 13 aa, Artificial Sequence
  Fc region binding polypeptide
KYWCQQWGVCNGS

SEQ ID NO: 61 - protein, 13 aa, Artificial Sequence
  Fc region binding polypeptide
KYWCVQWGVCPES

SEQ ID NO: 62 - protein, 13 aa, Artificial Sequence
  Fc region binding polypeptide
KYWCMQWGLCGWE

SEQ ID NO: 63 - protein, 13 aa, Artificial Sequence
  Fc region binding polypeptide
HFWCEVWGLCPSI

SEQ ID NO: 64 - protein, 13 aa, Artificial Sequence
  Fc region binding polypeptide
QYWCTKWGLCTNV

SEQ ID NO: 65 - protein, 13 aa, Artificial Sequence
  Fc region binding polypeptide
AYWCKVWGLCQGE

SEQ ID NO: 66 - protein, 13 aa, Artificial Sequence
  Fc region binding polypeptide
KYWCNLWGVCPAN

SEQ ID NO: 67 - protein, 13 aa, Artificial Sequence
  Fc region binding polypeptide
QYWCNVWGVCLPS

SEQ ID NO: 68 - protein, 13 aa, Artificial Sequence
  Fc region binding polypeptide
HYWCQQWGICERP

SEQ ID NO: 69 - protein, 13 aa, Artificial Sequence
  Fc region binding polypeptide
RYWCNIWDVCPEQ

SEQ ID NO: 70 - protein, 13 aa, Artificial Sequence
  Fc region binding polypeptide
QYWCTHWGLCGKY

SEQ ID NO: 71 - protein, 13 aa, Artificial Sequence
  Fc region binding polypeptide
TYWCTKWGLCPHN

SEQ ID NO: 72 - protein, 13 aa, Artificial Sequence
  Fc region binding polypeptide
FYWCGQWGLCAPP

SEQ ID NO: 73 - protein, 13 aa, Artificial Sequence
  Fc region binding polypeptide
GYWCNVWGLCSTE

SEQ ID NO: 74 - protein, 13 aa, Artificial Sequence
  Fc region binding polypeptide
RYWCGVWGVCEID

SEQ ID NO: 75 - protein, 13 aa, Artificial Sequence
  Fc region binding polypeptide
KFWCTIWGVCHMP

SEQ ID NO: 76 - protein, 13 aa, Artificial Sequence
  Fc region binding polypeptide
HYWCQQWGICERP

SEQ ID NO: 77 - protein, 13 aa, Artificial Sequence
  Fc region binding polypeptide
RYWCNIWDVCPEQ

SEQ ID NO: 78 - protein, 13 aa, Artificial Sequence
  Fc region binding polypeptide
FYWCSQWGLCKYD

SEQ ID NO: 79 - protein, 13 aa, Artificial Sequence
  Fc region binding polypeptide
HYWCEKWGLCLMS

SEQ ID NO: 80 - protein, 13 aa, Artificial Sequence
  Fc region binding polypeptide
HYWCQKWGVCPTD

SEQ ID NO: 81 - protein, 13 aa, Artificial Sequence
  Fc region binding polypeptide
HYWCSLWGVCDIN

SEQ ID NO: 82 - protein, 12 aa, Artificial Sequence
  Fc region binding polypeptide
RFWCSAWGVCPA

SEQ ID NO: 83 - protein, 13 aa, Artificial Sequence
  Fc region binding polypeptide
SYWCKIWDVCPQS

SEQ ID NO: 84 - protein, 13 aa, Artificial Sequence
  Fc region binding polypeptide
QYWCSIWKVCPGR

SEQ ID NO: 85 - protein, 13 aa, Artificial Sequence
  Fc region binding polypeptide
YWYCEWFGACIND

SEQ ID NO: 86 - protein, 14 aa, Artificial Sequence
  Fc region binding polypeptide
EYWCKYWGLECVHR

SEQ ID NO: 87 - protein, 14 aa, Artificial Sequence
  Fc region binding polypeptide
KYWCTQWGLKCDKQ

SEQ ID NO: 88 - protein, 13 aa, Artificial Sequence
  Fc region binding polypeptide
KYWCSFWGLQCKT

SEQ ID NO: 89 - protein, 14 aa, Artificial Sequence
  Fc region binding polypeptide
RYWCNFWGVNCDAN

SEQ ID NO: 90 - protein, 14 aa, Artificial Sequence
  Fc region binding polypeptide
NYWCTHWGVMCLDH

SEQ ID NO: 91 - protein, 14 aa, Artificial Sequence
  Fc region binding polypeptide
YWFCKWFPSQCQFM

SEQ ID NO: 92 - protein, 14 aa, Artificial Sequence
  Fc region binding polypeptide
AYWCKQWGLKCQLG

SEQ ID NO: 93 - protein, 14 aa, Artificial Sequence
  Fc region binding polypeptide
KYWCKFWGLECKVG

SEQ ID NO: 94 - protein, 14 aa, Artificial Sequence
  Fc region binding polypeptide
NYWCTEWGLNCNNK

SEQ ID NO: 95 - protein, 14 aa, Artificial Sequence
  Fc region binding polypeptide
SYWCEKWGLTCETH

SEQ ID NO: 96 - protein, 14 aa, Artificial Sequence
  Fc region binding polypeptide
EYWCRIWGLQCNMV

SEQ ID NO: 97 - protein, 14 aa, Artificial Sequence
  Fc region binding polypeptide
KYWCKKWGVNCDFN

SEQ ID NO: 98 - protein, 14 aa, Artificial Sequence
  Fc region binding polypeptide
KYWCSVWGVQCPHS

SEQ ID NO: 99 - protein, 14 aa, Artificial Sequence
  Fc region binding polypeptide
FYWCTKWGLECIHS

SEQ ID NO: 100 - protein, 14 aa, Artificial Sequence
  Fc region binding polypeptide
HYWCQQWGLMCFET

SEQ ID NO: 101 - protein, 14 aa, Artificial Sequence
  Fc region binding polypeptide
KYWCKRWGLMCNGG

SEQ ID NO: 102 - protein, 14 aa, Artificial Sequence
  Fc region binding polypeptide
AYWCMTWGVPCISW

SEQ ID NO: 103 - protein, 14 aa, Artificial Sequence
  Fc region binding polypeptide
KYWCKKWGVNCDFN

SEQ ID NO: 104 - protein, 14 aa, Artificial Sequence
  Fc region binding polypeptide
KYWCSVWGVQCPDS

SEQ ID NO: 105 - protein, 14 aa, Artificial Sequence
  Fc region binding polypeptide
KYWCSVWGVQCPHS

SEQ ID NO: 106 - protein, 14 aa, Artificial Sequence
  Fc region binding polypeptide
LYWCTKWGVTCQKD

SEQ ID NO: 107 - protein, 14 aa, Artificial Sequence
  Fc region binding polypeptide
TYWCHKWGVKCATT

SEQ ID NO: 108 - protein, 14 aa, Artificial Sequence
  Fc region binding polypeptide
TYWCTFWELPCDPA

SEQ ID NO: 109 - protein, 14 aa, Artificial Sequence
  Fc region binding polypeptide
KYWCTKWQLNCEEV

SEQ ID NO: 110 - protein, 14 aa, Artificial Sequence
  Fc region binding polypeptide
NYWCHFWQVPCLEQ

SEQ ID NO: 111 - protein, 14 aa, Artificial Sequence
  Fc region binding polypeptide
TYWCVVWNVPCSTD

SEQ ID NO: 112 - protein, 14 aa, Artificial Sequence
  Fc region binding polypeptide
NFWCHTWGLQCNDL

SEQ ID NO: 113 - protein, 14 aa, Artificial Sequence
  Fc region binding polypeptide
FWYCYWFNEKCKTP

SEQ ID NO: 114 - protein, 14 aa, Artificial Sequence
  Fc region binding polypeptide
GFWCTFWGVTCEAG

SEQ ID NO: 115 - protein, 14 aa, Artificial Sequence
  Fc region binding polypeptide
PHNCDDHYWYCKWF

SEQ ID NO: 116 - protein, 14 aa, Artificial Sequence
  Fc region binding polypeptide
EMTCSSHYWYCTWM

SEQ ID NO: 117 - protein, 14 aa, Artificial Sequence
  Fc region binding polypeptide
HIDCKTNYWWCRWT

SEQ ID NO: 118 - protein, 14 aa, Artificial Sequence
  Fc region binding polypeptide
EMRCGQHFWYCEWF

SEQ ID NO: 119 - protein, 15 aa, Artificial Sequence
  Fc region binding polypeptide
NYWCNFWQLPTCDNL

SEQ ID NO: 120 - protein, 16 aa, Artificial Sequence
  Fc region binding polypeptide
YWYCQWFQEVNKCFNS

SEQ ID NO: 121 - protein, 16 aa, Artificial Sequence
  Fc region binding polypeptide
YYWCRHWFPDFDCVHS

SEQ ID NO: 122 - protein, 16 aa, Artificial Sequence
  Fc region binding polypeptide
YWYCSWFPDRPDCPLY

SEQ ID NO: 123 - protein, 16 aa, Artificial Sequence
  Fc region binding polypeptide
YWYCVWFDNADQCVHH

SEQ ID NO: 124 - protein, 18 aa, Artificial Sequence
  Fc region binding polypeptide
AATCSTSYWYYQWFCTDS

SEQ ID NO: 125 - protein, 14 aa, Artificial Sequence
  Fc region binding polypeptide
YWACVWGLKSCVDR

SEQ ID NO: 126 - protein, 13 aa, Artificial Sequence
  Fc region binding polypeptide
YWRCVWFPASCPT

SEQ ID NO: 127 - protein, 18 aa, Artificial Sequence
  Fc region binding polypeptide
DWQCLWWGNSFWPYCANL

SEQ ID NO: 128 - protein, 14 aa, Artificial Sequence
  Fc region binding polypeptide
FWRCHWWPERCPVD

SEQ ID NO: 129 - protein, 18 aa, Artificial Sequence
  Fc region binding polypeptide
NPMCWKKSWWEDAYCINH

SEQ ID NO: 130 - protein, 18 aa, Artificial Sequence
  Fc region binding polypeptide
SWVCWKAKWWEDKRCAPF

SEQ ID NO: 131 - protein, 18 aa, Artificial Sequence
  Fc region binding polypeptide
SRQCWKELWWTDQMCLDL

SEQ ID NO: 132 - protein, 16 aa, Artificial Sequence
  Fc region binding polypeptide
SFRCQSSFPSWYCDYY

SEQ ID NO: 133 - protein, 16 aa, Artificial Sequence
  Fc region binding polypeptide
SWHCQNTYPEWYCQWY

SEQ ID NO: 134 - protein, 17 aa, Artificial Sequence
  Fc region binding polypeptide
GSKCKQTGFPRWWCEHY

SEQ ID NO: 135 - protein, 18 aa, Artificial Sequence
  Fc region binding polypeptide
DGVCGPRGFGPAWFCMHY

SEQ ID NO: 136 - protein, 16 aa, Artificial Sequence
  Fc region binding polypeptide
YSHCATHYPTWYCLHF

SEQ ID NO: 137 - protein, 16 aa, Artificial Sequence
  Fc region binding polypeptide
FCNCWGSHEFTFCVDD

SEQ ID NO: 138 - protein, 18 aa, Artificial Sequence
  Fc region binding polypeptide
PGWCYSDIWGFKHFCNLD

SEQ ID NO: 139 - protein, 16 aa, Artificial Sequence
  Fc region binding polypeptide
DSSCIKHHNKVTCFFP

SEQ ID NO: 140 - protein, 13 aa, Artificial Sequence
  Fc region binding polypeptide
RWSCWGVWGCVWV

SEQ ID NO: 141 - protein, 14 aa, Artificial Sequence
  Fc region binding polypeptide
PVDCKHHFWWCYWN

SEQ ID NO: 142 - protein, 17 aa, Artificial Sequence
  Fc region binding polypeptide
SWNCAFHHNEMVWCDDG

SEQ ID NO: 143 - protein, 15 aa, Artificial Sequence
  Fc region binding polypeptide
YWYCWFPDRPECPLY

SEQ ID NO: 144 - protein, 29 aa, Artificial Sequence
  Fc region binding polypeptide
GDDHMCVYTTWGELIWCDNHEPGPEGGGK

SEQ ID NO: 145 - protein, 29 aa, Artificial Sequence
  Fc region binding polypeptide
GDRRACSRDWSGALVWCAGHEPGPEGGGK

SEQ ID NO: 146 - protein, 25 aa, Artificial Sequence
  Fc region binding polypeptide
AGKYWCSFWGLQCKTGTPGPEGGGK

SEQ ID NO: 147 - protein, 23 aa, Artificial Sequence
  Fc region binding polypeptide
AGSYWCKIWDVCPQSPGPEGGGK

SEQ ID NO: 148 - protein, 23 aa, Artificial Sequence
  Fc region binding polypeptide
AGKYWCNLWGVCPANPGPEGGGK

SEQ ID NO: 149 - protein, 24 aa, Artificial Sequence
  Fc region binding polypeptide
AGTYWCTFWELPCDPAPGPEGGGK

SEQ ID NO: 150 - protein, 24 aa, Artificial Sequence
  Fc region binding polypeptide
AGPHNCDDHYWYCKWFPGPEGGGK

SEQ ID NO: 151 - protein, 28 aa, Artificial Sequence
  Fc region binding polypeptide
AGAATCSTSYWYYQWFCTDSPGPEGGGK

SEQ ID NO: 152 - protein, 25 aa, Artificial Sequence
  Fc region binding polypeptide
AGYWYCWFPDRPECPLYPGPEGGGK

SEQ ID NO: 153 - protein, 26 aa, Artificial Sequence
  Fc region binding polypeptide
AGPVDCKHHFWWCYWNGTPGPEGGGK

SEQ ID NO: 154 - protein, 29 aa, Artificial Sequence
  Fc region binding polypeptide
GDDDHCYWFREWFNSECPHGEPGPEGGGK

SEQ ID NO: 155 - protein, 25 aa, Artificial Sequence
  Fc region binding polypeptide
AGYYWCNYWGLCPDQGTPGPEGGGK

SEQ ID NO: 156 - protein, 30 aa, Artificial Sequence
  Fc region binding polypeptide
GDSWVCWKAKWWEDKRCAPFGTPGPEGGGK

SEQ ID NO: 157 - protein, 30 aa, Artificial Sequence
  Fc region binding polypeptide
GDNPMCWKKSWWEDAYCINHGTPGPEGGGK

SEQ ID NO: 158 - protein, 29 aa, Artificial Sequence
  Fc region binding polypeptide
GDSWNCAFHHNEMVWCDDGGTPGPEGGGK

SEQ ID NO: 159 - protein, 29 aa, Artificial Sequence
  Fc region binding polypeptide
GDWGECTVTSYGELIWCGGLEPGPEGGGK

SEQ ID NO: 160 - protein, 29 aa, Artificial Sequence
  Fc region binding polypeptide
GDNPMCWRASWWEDAYCINHEPGPEGGGK

SEQ ID NO: 161 - protein, 29 aa, Artificial Sequence
  Fc region binding polypeptide
GDNPMCWRAHWWEDAYCINHEPGPEGGGK

SEQ ID NO: 162 - protein, 26 aa, Artificial Sequence
  Fc region binding polypeptide
GDDHMCVYTTWGELIWCDNHEPGPEG

SEQ ID NO: 163 - protein, 23 aa, Artificial Sequence
  Fc region binding polypeptide
GDDHMCVYTTWGELIWCDNHEPG

SEQ ID NO: 164 - protein, 23 aa, Artificial Sequence
  Fc region binding polypeptide
GDDHMCVYTTWGELIWCDNHEPG

SEQ ID NO: 165 - protein, 20 aa, Artificial Sequence
  Fc region binding polypeptide
GDDHMCVYTTWGELIWCDNH

SEQ ID NO: 166 - protein, 20 aa, Artificial Sequence
  Fc region binding polypeptide
GDDHMCVYTTWGELIWCDNH

SEQ ID NO: 167 - protein, 20 aa, Artificial Sequence
  Fc region binding polypeptide
GDDHMCVYTTWGELIWCDNH

SEQ ID NO: 168 - protein, 6 aa, Artificial Sequence
  c-terminal linker peptide
PEGGGK

SEQ ID NO: 169 - protein, 27 aa, Artificial Sequence
  Fc region binding polypeptide
DHMCVYTTWGELIWCDNHEPGPEGGGK

SEQ ID NO: 170 - protein, 27 aa, Artificial Sequence
  Fc region binding polypeptide
EHMCVYTTWGELIWCDNHEPGPEGGGK

SEQ ID NO: 171 - protein, 25 aa, Artificial Sequence
  Fc region binding polypeptide
ACVYTTWGELIWCDNHEPGPEGGGK

SEQ ID NO: 172 - protein, 25 aa, Artificial Sequence
  Fc region binding polypeptide
TCVYTTWGELIWCDNHEPGPEGGGK

SEQ ID NO: 173 - protein, 25 aa, Artificial Sequence
  Fc region binding polypeptide
ECVYTTWGELIWCDNHEPGPEGGGK

SEQ ID NO: 174 - protein, 25 aa, Artificial Sequence
  Fc region binding polypeptide
VCVYTTWGELIWCDNHEPGPEGGGK

SEQ ID NO: 175 - protein, 24 aa, Artificial Sequence
  Fc region binding polypeptide
CVYTTWGELIWCDNHEPGPEGGGK

SEQ ID NO: 176 - protein, 24 aa, Artificial Sequence
  Fc region binding polypeptide
CVYTTWGELIWCDNHEPGPEGGGK

SEQ ID NO: 177 - protein, 27 aa, Artificial Sequence
  Fc region binding polypeptide
SRACSRDWSGALVWCAGHEPGPEGGGK

SEQ ID NO: 178 - protein, 27 aa, Artificial Sequence
  Fc region binding polypeptide
RRACSRDWSGALVWCAGHEPGPEGGGK

SEQ ID NO: 179 - protein, 27 aa, Artificial Sequence
  Fc region binding polypeptide
ERACSRDWSGALVWCAGHEPGPEGGGK

SEQ ID NO: 180 - protein, 25 aa, Artificial Sequence
  Fc region binding polypeptide
ACSRDWSGALVWCAGHEPGPEGGGK

SEQ ID NO: 181 - protein, 25 aa, Artificial Sequence
  Fc region binding polypeptide
TCSRDWSGALVWCAGHEPGPEGGGK

SEQ ID NO: 182 - protein, 25 aa, Artificial Sequence
  Fc region binding polypeptide
ECSRDWSGALVWCAGHEPGPEGGGK

SEQ ID NO: 183 - protein, 25 aa, Artificial Sequence
  Fc region binding polypeptide
VCSRDWSGALVWCAGHEPGPEGGGK

SEQ ID NO: 184 - protein, 25 aa, Artificial Sequence
  Fc region binding polypeptide
GCSRDWSGALVWCAGHEPGPEGGGK

SEQ ID NO: 185 - protein, 24 aa, Artificial Sequence
  Fc region binding polypeptide
CSRDWSGALVWCAGHEPGPEGGGK

SEQ ID NO: 186 - protein, 18 aa, Artificial Sequence
  Fc region binding polypeptide
NPMCWRASWWEDAYCINH

SEQ ID NO: 187 - protein, 18 aa, Artificial Sequence
  Fc region binding polypeptide
NPMCWRAHWWEDAYCINH

SEQ ID NO: 188 - protein, 18 aa, Artificial Sequence
  Fc region binding polypeptide
EHMCVYTTWGELIWCDNH

SEQ ID NO: 189 - protein, 16 aa, Artificial Sequence
  Fc region binding polypeptide
ACVYTTWGELIWCDNH

SEQ ID NO: 190 - protein, 16 aa, Artificial Sequence
  Fc region binding polypeptide
TCVYTTWGELIWCDNH

SEQ ID NO: 191 - protein, 16 aa, Artificial Sequence
  Fc region binding polypeptide
ECVYTTWGELIWCDNH

SEQ ID NO: 192 - protein, 16 aa, Artificial Sequence
  Fc region binding polypeptide
VCVYTTWGELIWCDNH

SEQ ID NO: 193 - protein, 15 aa, Artificial Sequence
  Fc region binding polypeptide
CVYTTWGELIWCDNH

SEQ ID NO: 194 - protein, 16 aa, Artificial Sequence
  Fc region binding polypeptide
SRACSRDWSGALVWCA

SEQ ID NO: 195 - protein, 18 aa, Artificial Sequence
  Fc region binding polypeptide
ERACSRDWSGALVWCAGH

SEQ ID NO: 196 - protein, 16 aa, Artificial Sequence
  Fc region binding polypeptide
ACSRDWSGALVWCAGH

SEQ ID NO: 197 - protein, 16 aa, Artificial Sequence
  Fc region binding polypeptide
TCSRDWSGALVWCAGH

SEQ ID NO: 198 - protein, 16 aa, Artificial Sequence
  Fc region binding polypeptide
ECSRDWSGALVWCAGH

SEQ ID NO: 199 - protein, 16 aa, Artificial Sequence
  Fc region binding polypeptide
VCSRDWSGALVWCAGH

SEQ ID NO: 200 - protein, 16 aa, Artificial Sequence
  Fc region binding polypeptide
GCSRDWSGALVWCAGH

(19) United States
(12) Patent Application Publication
(10) Pub. No.: US 2003/0199671 A1
(43) Pub. Date: Oct. 23, 2003

(54) Binding molecules for Fc-region polypeptides

(76) Inventors: Isaac Jesus Rondon, Boston, MA (US); Qi-Long Wu, Brighton, MA (US); Arthur C. Ley, Newton, MA (US); Mark Stochl, N. Attleboro, MA (US); Thomas C. Ransohoff, Lexington, MA (US); M. Daniel Potter, Acton, MA (US); Marilou Potter, Acton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/125,869

(22) Filed: Apr. 18, 2002

Related U.S. Application Data
(60) Provisional application No. 60/284,534, filed on Apr. 18, 2001.

(51) Int. Cl. C07K 7/64

(52) U.S. Cl. 530/317

(57) ABSTRACT

Binding molecules for detecting or isolating immunoglobulin Fc-region polypeptides are described, together with methods of use thereof. Preferred Fc-region binding polypeptides, recombinant bacteriophage expressing Fc-region binding polypeptides, and separation media exhibiting such polypeptides are particularly disclosed.

### RELATED APPLICATION

This application claims priority under 35 U.S.C. §119 to U.S. provisional application no. 60/284,534, filed Apr. 18, 2001.

### FIELD OF THE INVENTION

The present invention relates to the field of protein isolation and purification. Specifically, the present invention relates to the identification, isolation, and synthesis of molecules that bind to human immunoglobulin Fc-region polypeptides. Such binding molecules are useful for the detection, removal, or purification of isolated Fc-region amino acid sequences or polypeptides such as immunoglobulins or fusion proteins having an Fc region present as one domain of the polypeptide.

### BACKGROUND

Antibodies (immunoglobulins) are a group of glycoproteins produced by B lymphocytes in response to the presence of a foreign antigen and are present in the serum and tissue fluids of all mammals. Each antibody molecule is essentially bifunctional: one region of the molecule, Fab, contains the antigen binding site(s), and a second region, Fc (for “fragment crystallizable”), can contain effector sequences that, for instance, facilitate the binding of the antibody molecule to Fc receptors located on the surface of various cells of the immune system, such as macrophages and neutrophils.

It has been shown that the Fc regions of various immunoglobulins, particularly IgG, possess an affinity for Staphyloccal Protein A (Ey et al., Immunochemistry, 15: 429-436 (1978)), and several methods have been developed to exploit this interaction to purify both polyclonal and monoclonal antibodies. For instance, Ngo et al., U.S. Pat. No. 4,801,687, describes a method for the purification of a monoclonal antibody from a fluid (e.g., serum) by contacting the fluid with Protein A immobilized on an affinity column. Ngo et al. demonstrated that IgG, in a buffer containing a combination of monovalent cations and polybasic anions, forms a binding complex with Protein A under conditions of high pH (7.5-10) and will dissociate when contacted with a solution of low pH, i.e., pH 3-6. Shadle et al., U.S. Pat. No. 5,429,746, purified an IgG utilizing a hydrophobic interaction chromatography (HIC) protocol or a multistep procedure utilizing a Protein A affinity step, followed by an ion exchange step, followed by a HIC step.

However, Protein A does not discriminate between IgG's from different species. Thus, to develop transgenic models for the production of human-specific immunoglobulin ligands, it is essential to develop ligands that bind human immunoglobulins specifically, and not any of the immunoglobulins of the transgenic host.

However, other antibody purification methods have been developed that do not require the use of Protein A. For example, Crane et al., U.S. Pat. No. 4,606,825 describes the purification of IgG from a biological fluid by a liquid column chromatography procedure utilizing a silica gel to bind the immunoglobulin followed by elution in a buffer having a pH of 5.5-8.3. Sullivan et al., U.S. Pat. No. 4,849,352 describes the purification of Fab and F(ab)₂ fragments by affinity chromatography using polyacrylamide gels; and Raison et al., U.S. Pat. No. 5,077,391 describes the purification of IgM by complexing with immobilized Clq, a protein involved in the complement pathway.

Another antibody purification method is described by Hakalahti et al., J. Immunol. Meth., 117: 131-136 (1989), utilizing a protocol employing two successive ion exchange chromatographic steps or a single ion exchange step followed by a hydrophobic interaction chromatography (HIC) step.

However, a disadvantage of these antibody purification methods is the requirement for a multiple binding domain protein affinity ligand, Protein A, and/or multiple purification columns. For instance, Ibrahim, Scand. J. Immunol., 38: 368-374 (1993), reported that Protein A has a total of five homology domains (A-E) and that up to four of these domains bind the Fc region of IgG.

Dybwad et al., Clin. Exp. Immunol., 102: 438 (1995) examined the potential for isolating ligands for polyclonal antibodies using a phage display library. The library was contacted with rabbit antisera raised against a synthetic 7-mer polypeptide. Dybwad et al. reported that the sequence of many of the positive phage demonstrated high sequence homology to the 17-mer peptide. Fassina et al., J. Mol. Recognit., 9: 564 (1996) screened a synthetic multimeric peptide library to identify a Protein A mimetic peptide possessing the ability to compete with the interaction between Protein A and biotinylated immunoglobulins. Ehrlich et al., J. Mol. Recognit., 11: 121-125 (1998) treated a humanized IgG1 monoclonal antibody with pepsin to separate pFc′ fragments from the rest of the antibody. The pFc′ fragments were used as targets for a variable-length phage display library to identify peptides with an affinity for the pFc′ fragments.

Krooket al., J. Immunol. Meth., 221: 151-157 (1998) screened a decapeptide phage display library using IgG Fc as a target to identify linear 10-mer peptide sequences as possible analogs to Protein A or for affinity ligands to parts of the Fc molecule not involved in Protein A binding. DeLano et al., Science, 287: 1279 (2000), screened a phage library displaying cyclic peptides of varying sizes using IgG Fc as a target sequence. DeLano et al. reported that after several rounds of screening, the recovered Fc region binding peptides were dominated by two polypeptide sequences, ETQRCTWHMGELVWCEREHN (SEQ ID NO: 1) and KEASCSYWLGELVWCVAGVE (SEQ ID NO: 2). Thus, DeLano et al. described two cyclic 11-mers having a common GELVWC (SEQ ID NO:3) motif. However, Delano et al. did not demonstrate chromatographic capture and recovery of purified human immunoglobulins.

There is still a need for additional peptide ligands that would provide improved ligands for Fc-region polypeptides and improved purification methods.

### SUMMARY OF THE INVENTION

The Fc-region binding molecules of the present invention were isolated utilizing phage display libraries in which the display comprised a variegated peptide of from 12-18 amino acids, which peptide included two invariant cysteines, spaced to provide (upon formation of a disulfide bond between the cysteines) cyclic peptides having 6, 7, 8, 9, 10, or 12 amino acid residues. The newly discovered Fc-region binding molecules have a highly specific affinity for the antibody Fc region. For separation applications, it is preferred that the ligand have an affinity for the target that is in the range 10 μM to 0.01 μM and much less affmity for non-target materials. “High affinity” as used herein will refer to binding moieties having a K_{D} in the range of about 10 μM to about 0.01 μM with respect to a target material, with much less affinity for non-target materials. When recovery of the target material is desired, it is also important that the immobilized ligand have negligible affinity for the target under a set of conditions that does not denature the target; otherwise, it is impossible to recover the target from the immobilized ligand. In addition to being advantageous for the rapid and efficient isolation of antibodies in highly purified yields, the present invention also discloses the use of these novel polypeptides for the rapid purification of a protein of interest expressed as part of a fusion protein comprising an Fc-region polypeptide.

Accordingly, it is an object of the present invention to provide novel binding molecules for human Fc-region polypeptides. Preferred binding molecules of the present invention exhibit not only distinct characteristics for binding to the target Fc-region amino acid sequence but also specific characteristics for the release (elution) of the target Fc-region binding molecules. Especially preferred binding molecules according to the invention are short polypeptide sequences, characterized by a stable loop structure (i.e., cyclic peptides).

A preferred method is disclosed herein for isolation of binding molecules according to the invention by employing phage display technology. The phage display method of the current invention is useful for identifying families of polypeptide binding molecules. Using this technique, a number of binding peptides exhibiting affinity for human Fc-region amino acid sequences have been identified and isolated. Such binding peptides are useful for identifying, isolating and purifying human Fc-region polypeptides present in a solution (e.g., whole blood, plasma, transgenic milk, eggs of transgenic birds (such as chickens, quail, turkeys, ostrich, or geese), conditioned media, etc.) as isolated polypeptides, for isolating and purifying antibodies, and for isolating and purifying fusion proteins genetically engineered to include Fc-region amino acid sequences. Transgenic milk can be obtained from transgenic mice, rats, rabbits, goats, sheep, and cows.

The most preferred binding molecules specific for Fc-region polypeptides include two spaced, invariant cyteine residues and are thus capable of forming a cyclic strucure under non-reducing conditions via a disulfide bond formed between the cysteine side chains. Specific Fc-region binding molecules according to the present invention include polypeptides comprising amino acid sequences of the following four general formulas:

Z₁—X₁—X₂—X₃—X₄—W—C—Z₂(SEQ ID NO:4); I.

wherein,

Z₁ is a polypeptide of at least 6 amino acids;

X₁ is G, H, N, R, or S;

X₂ is A, D, E, F, I, M, or S;

X₃ is A, I, L, M, or V;

X₄ is I, M, T, or V;

Z₂ is a polypeptide of at least one amino acid or is absent; and

Z₁ contains at least one cysteine residue such that formation of a disulfide bond with the invariant cysteine residue forms a cyclic peptide of 12 amino acids.

Z₁—X—W—Z₂—W—Z₃(SEQ ID NO:5) II.

wherein,

Z₁ is a polypeptide of at least one amino acid or is absent;

X is F or Y;

Z₂ is a tripeptide; and

Z₃ is a polypeptide of at least one amino acid; and

wherein at least two of the polypeptides Z₁, Z₂, and Z₃ contain a cysteine residue, such that formation of a disulfide bond between such cysteine residues forms a cyclic peptide of 7-12 amino acids.

In the foregoing formula II polypeptides, it is preferred that Z₂ has the formula (IIA):

X₁—X₂—X₃ (IIA),

wherein,

X₁ is A, C, F, K, P, R, W, or Y;

X₂ is C, D, E, G, H, K, M, N, Q, R, S, T, V, or Y; and

X₃ is A, E, F, H, I, K, L, Q, R, S, T, V, or Y;

with the proviso that at most one of X₁, X₂ and X₃ can be C. Preferably, where X₂ is C, then X₁ is Y. Most preferably, X₁ is C.

Z₁—W—Z₂—W—W—Z₃(SEQ ID NO:6); III.

wherein,

Z₁ is a polypeptide of at least one amino acid;

Z₂ is a tripeptide; and

Z₃ is a polypeptide of at least one amino acid;

wherein at least two of the polypeptides Z₁, Z₂, and Z₃ contain a cysteine residue, such that formation of a disulfide bond between such cysteine residues forms a cyclic peptide of 8-12 amino acids, with the proviso that where Z₁ contains a cysteine, then Z₂ does not contain a cysteine, and where Z₂ contains a cysteine, it is the middle residue of the tripeptide and Z₃ also contains a cysteine.

Preferably for the polypeptides of formula III, when Z₁ and Z₃ each contain a cysteine residue, the cysteine of Z₁ is adjacent the invariant tryptophan (W), the first amino acid of Z₂ is lysine and the second amino acid of Z₃ is apartic acid (D).

Z₁—P—X₁—W—X₂—C—X₃—X₄—X₅(SEQ ID NO:7); IV.

wherein,

Z₁ is a polypeptide of at least one amino acid and includes a cysteine residue;

X₁ is A, E, R, S, or T;

X₂ is F, W, or Y;

X₃ is D, E, L, M, or Q;

X₄ is H, W, or Y;

X₅ is F or Y; and

wherein the cysteine residue in Z₁ and the cysteine residue between X₂ and X₃ form a cyclic peptide of 10-12 amino acids.

Fc-region binding polypeptides of the present invention include polypeptides comprising amino acid sequences selected from the group consisting of:

(SEQ ID NO:14)

R-R-A-C-S-R-D-W-S-G-A-L-V-W-C-A-G-H

(SEQ ID NO:15)

W-G-E-C-T-V-T-S-Y-G-E-L-I-W-C-G-G-L

(SEQ ID NO:16)

S-S-A-C-A-F-D-P-M-G-A-V-I-W-C-T-Y-D

(SEQ ID NO:17)

L-L-E-C-A-Y-N-T-S-G-E-L-I-W-C-N-G-S

(SEQ ID NO:18)

P-D-D-C-S-I-H-F-S-G-E-L-I-W-C-E-P-L

(SEQ ID NO:19)

L-G-E-C-T-V-T-S-Y-G-E-L-I-W-C-G-G-L

(SEQ ID NO:20)

W-G-E-C-T-V-T-S-Y-G-E-L-I-W-C-G-G-H

(SEQ ID NO:21)

D-H-M-C-V-Y-T-T-W-G-E-L-I-W-C-D-D-H

(SEQ ID NO:22)

W-G-E-C-T-V-T-S-Y-G-E-L-I-W-C-G-G-L

(SEQ ID NO:23)

C-R-A-C-S-R-D-W-P-G-A-L-V-W-C-A-G-H

(SEQ ID NO:24)

R-R-A-C-S-R-D-W-S-G-A-L-V-W-C-A-G-H

(SEQ ID NO:25)

L-H-A-C-A-F-D-P-M-G-A-V-I-W-C-T-Y-D

(SEQ ID NO:26)

D-H-M-C-V-Y-T-T-W-G-E-L-M-W-C-D-N-H

(SEQ ID NO:27)

P-P-T-C-T-W-D-W-Q-G-I-L-V-W-C-S-G-H

(SEQ ID NO:28)

S-N-K-C-S-N-T-W-D-G-S-L-I-W-C-S-A-N

(SEQ ID NO:29)

F-P-E-C-T-F-D-M-E-G-F-L-I-W-C-S-S-F

(SEQ ID NO:30)

H-D-L-C-A-Q-A-P-F-G-D-A-T-W-C-D-L-R

(SEQ ID NO:31)

P-N-H-C-S-Y-N-L-K-S-E-L-I-W-C-Q-D-L

(SEQ ID NO:32)

P-L-D-C-A-R-D-I-H-N-S-L-I-W-C-S-L-G

(SEQ ID NO:33)

G-S-E-C-S-W-T-S-L-N-E-L-I-W-C-A-H-W

(SEQ ID NO:34)

W-P-D-C-S-F-T-V-Q-R-D-L-I-W-C-E-A-L

(SEQ ID NO:35)

S-H-S-C-A-Y-D-Y-A-H-M-L-V-W-C-T-H-F

(SEQ ID NO:36)

D-H-M-C-V-Y-T-T-W-G-E-L-I-W-C-D-N-H

(SEQ ID NO:37)

R-P-N-C-T-F-A-A-S-G-E-L-I-W-C-M-H-Y

(SEQ ID NO:38)

W-W-G-C-Q-F-D-W-R-G-E-L-V-W-C-P-Y-L

(SEQ ID NO:39)

G-G-V-C-S-Y-S-G-M-G-E-I-V-W-C-R-W-F

(SEQ ID NO:40)

A-L-M-C-S-H-D-M-W-G-S-L-I-W-C-K-H-F

(SEQ ID NO:41)

W-W-N-C-H-N-G-W-T-W-T-G-G-W-C-W-W-F

(SEQ ID NO:42)

Y-H-V-C-A-R-D-S-W-D-Q-L-I-W-C-E-A-F

(SEQ ID NO:43)

N-Y-W-C-N-F-W-Q-L-P-T-C-D-N-L

(SEQ ID NO:44)

Y-W-Y-C-K-W-F-S-E-S-A-S-C-S-S-R

(SEQ ID NO:45)

Y-W-Y-C-K-W-F-E-D-K-H-P-C-D-S-S

(SEQ ID NO:46)

Y-W-Y-C-S-W-F-P-D-R-P-D-C-P-L-Y

(SEQ ID NO:47)

N-Y-W-C-N-V-W-L-L-G-D-V-C-R-S-H

(SEQ ID NO:48)

L-Y-W-C-H-V-W-F-G-Q-H-A-W-Q-C-K-Y-P

(SEQ ID NO:49)

Y-W-K-C-K-W-M-P-W-M-C-G-F-D

(SEQ ID NO:50)

D-D-H-C-Y-W-F-R-E-W-F-N-S-E-C-P-H-G

(SEQ ID NO:51)

N-Y-W-C-N-I-W-G-L-H-G-C-N-S-H

(SEQ ID NO:52)

Y-W-F-C-Q-W-F-S-Q-N-H-T-C-F-R-D

(SEQ ID NO:53)

H-Y-W-C-D-I-W-F-G-A-P-A-C-Q-F-R

(SEQ ID NO:54)

S-G-D-C-G-F-W-P-R-I-W-G-L-C-M-D-N

(SEQ ID NO:55)

F-W-Y-C-K-W-F-Y-E-D-A-Q-C-S-H-D

(SEQ ID NO:56)

Y-Y-W-C-N-Y-W-G-L-C-P-D-Q

(SEQ ID NO:57)

S-Y-W-C-K-I-W-D-V-C-P-Q-S

(SEQ ID NO:58)

K-Y-W-C-N-L-W-G-V-C-P-A-N

(SEQ ID NO:59)

Q-Y-W-C-Y-Q-W-G-L-C-G-A-N

(SEQ ID NO:60)

K-Y-W-C-Q-Q-W-G-V-C-N-G-S

(SEQ ID NO:61)

K-Y-W-C-V-Q-W-G-V-C-P-E-S

(SEQ ID NO:62)

K-Y-W-C-M-Q-W-G-L-C-G-W-E

(SEQ ID NO:63)

H-F-W-C-E-V-W-G-L-C-P-S-I

(SEQ ID NO:64)

Q-Y-W-C-T-K-W-G-L-C-T-N-V

(SEQ ID NO:65)

A-Y-W-C-K-V-W-G-L-C-Q-G-E

(SEQ ID NO:66)

K-Y-W-C-N-L-W-G-V-C-P-A-N

(SEQ ID NO:67)

Q-Y-W-C-N-V-W-G-V-C-L-P-S

(SEQ ID NO:68)

H-Y-W-C-Q-Q-W-G-I-C-E-R-P

(SEQ ID NO:69)

R-Y-W-C-N-I-W-D-V-C-P-E-Q

(SEQ ID NO:70)

Q-Y-W-C-T-H-W-G-L-C-G-K-Y

(SEQ ID NO:71)

T-Y-W-C-T-K-W-G-L-C-P-H-N

(SEQ ID NO:72)

F-Y-W-C-G-Q-W-G-L-C-A-P-P

(SEQ ID NO:73)

G-Y-W-C-N-V-W-G-L-C-S-T-E

(SEQ ID NO:74)

R-Y-W-C-G-V-W-G-V-C-E-I-D

(SEQ ID NO:75)

K-F-W-C-T-I-W-G-V-C-H-M-P

(SEQ ID NO:76)

H-Y-W-C-Q-Q-W-G-I-C-E-R-P

(SEQ TD NO:77)

R-Y-W-C-N-I-W-D-V-C-P-E-Q

(SEQ ID NO:78)

F-Y-W-C-S-Q-W-G-L-C-K-Y-D

(SEQ ID NO:79)

H-Y-W-C-E-K-W-G-L-C-L-M-S

(SEQ ID NO:80)

H-Y-W-C-Q-K-W-G-V-C-P-T-D

(SEQ ID NO:81)

H-Y-W-C-S-L-W-G-V-C-D-I-N

(SEQ ID NO:82)

R-F-W-C-S-A-W-G-V-C-P-A

(SEQ ID NO:83)

S-Y-W-C-K-I-W-D-V-C-P-Q-S

(SEQ ID NO:84)

Q-Y-W-C-S-I-W-K-V-C-P-G-R

(SEQ ID NO:85)

Y-W-Y-C-E-W-F-G-A-C-I-N-D

(SEQ ID NO:86)

E-Y-W-C-K-Y-W-G-L-E-C-V-H-R

(SEQ ID NO:87)

K-Y-W-C-T-Q-W-G-L-K-C-D-K-Q

(SEQ ID NO:88)

K-Y-W-C-S-F-W-G-L-Q-C-K-J

(SEQ ID NO:89)

R-Y-W-C-N-F-W-G-V-N-C-D-A-N

(SEQ ID NO:90)

N-Y-W-C-T-H-W-G-V-M-C-L-D-H

(SEQ ID NO:91)

Y-W-F-C-K-W-F-P-S-Q-C-Q-F-M

(SEQ ID NO:92)

A-Y-W-C-K-Q-W-G-L-K-C-Q-L-G

(SEQ ID NO:93)

K-Y-W-C-K-F-W-G-L-E-C-K-V-G

(SEQ ID NO:94)

N-Y-W-C-T-E-W-G-L-N-C-N-N-K

(SEQ ID NO:95)

S-Y-W-C-E-K-W-G-L-T-C-E-T-H

(SEQ ID NO:96)

E-Y-W-C-R-I-W-G-L-Q-C-N-M-V

(SEQ ID NO:97)

K-Y-W-C-K-K-W-G-V-N-C-D-F-N

(SEQ ID NO:98)

K-Y-W-C-S-V-W-G-V-Q-C-P-H-S

(SEQ ID NO:99)

F-Y-W-C-T-K-W-G-L-E-C-I-H-S

(SEQ ID NO:100)

H-Y-W-C-Q-Q-W-G-L-M-C-F-E-T

(SEQ ID NO:101)

K-Y-W-C-K-R-W-G-L-M-C-N-G-G

(SEQ ID NO:102)

A-Y-W-C-M-T-W-G-V-P-C-I-S-W

(SEQ ID NO:103)

K-Y-W-C-K-K-W-G-V-N-C-D-F-N

(SEQ ID NO:104)

K-Y-W-C-S-V-W-G-V-Q-C-P-D-S

(SEQ ID NO:105)

K-Y-W-C-S-V-W-G-V-Q-C-P-H-S

(SEQ ID NO:106)

L-Y-W-C-T-K-W-G-V-T-C-Q-K-D

(SEQ ID NO:107)

T-Y-W-C-H-K-W-G-V-K-C-A-T-T

(SEQ ID NO:108)

T-Y-W-C-T-F-W-E-L-P-C-D-P-A

(SEQ ID NO:109)

K-Y-W-C-T-K-W-Q-L-N-C-E-E-V

(SEQ ID NO:110)

N-Y-W-C-H-F-W-Q-V-P-C-L-E-Q

(SEQ ID NO:111)

T-Y-W-C-V-V-W-N-V-P-C-S-T-D

(SEQ ID NO:112)

N-F-W-C-H-T-W-G-L-Q-C-N-D-L

(SEQ ID NO:113)

F-W-Y-C-Y-W-F-N-E-K-C-K-T-P

(SEQ ID NO:114)

G-F-W-C-T-F-W-G-V-T-C-E-A-G

(SEQ ID NO:115)

P-H-N-C-D-D-H-Y-W-Y-C-K-W-F

(SEQ ID NO:116)

E-M-T-C-S-S-H-Y-W-Y-C-T-W-M

(SEQ ID NO:117)

H-I-D-C-K-T-N-Y-W-W-C-R-W-T

(SEQ ID NO:118)

E-M-R-C-G-Q-H-F-W-Y-C-E-W-F

(SEQ ID NO:119)

N-Y-W-C-N-F-W-Q-L-P-T-C-D-N-L

(SEQ ID NO:120)

Y-W-Y-C-Q-W-F-Q-E-V-N-K-C-F-N-S

(SEQ ID NO:121)

Y-Y-W-C-R-H-W-F-P-D-F-D-C-V-H-S

(SEQ ID NO:122)

Y-W-Y-C-S-W-F-P-D-R-P-D-C-P-L-Y

(SEQ ID NO:123)

Y-W-Y-C-V-W-F-D-N-A-D-Q-C-V-H-H

(SEQ ID NO:124)

A-A-T-C-S-T-S-Y-W-Y-Y-Q-W-F-C-T-D-S

(SEQ ID NO:125)

Y-W-A-C-V-W-G-L-K-S-C-V-D-R

(SEQ ID NO:126)

Y-W-R-C-V-W-F-P-A-S-C-P-T

(SEQ ID NO:127)

D-W-Q-C-L-W-W-G-N-S-F-W-P-Y-C-A-N-L

(SEQ ID NO:128)

F-W-R-C-H-W-W-P-E-R-C-P-V-D

(SEQ ID NO:129)

N-P-M-C-W-K-K-S-W-W-E-D-A-Y-C-I-N-H

(SEQ ID NO:130)

S-W-V-C-W-K-A-K-W-W-E-D-K-R-C-A-P-F

(SEQ ID NO:131)

S-R-Q-C-W-K-E-L-W-W-T-D-Q-M-C-L-D-L

(SEQ ID NO:132)

S-F-R-C-Q-S-S-F-P-S-W-Y-C-D-Y-Y

(SEQ ID NO:133)

S-W-H-C-Q-N-T-Y-P-E-W-Y-C-Q-W-Y

(SEQ ID NO:134)

G-S-K-C-K-Q-T-G-F-P-R-W-W-C-E-H-Y

(SEQ ID NO:135)

D-G-V-C-G-P-R-G-F-G-P-A-W-F-C-M-H-Y

(SEQ ID NO:136)

Y-S-H-C-A-T-H-Y-P-T-W-Y-C-L-H-F

(SEQ ID NO:137)

F-C-N-C-W-G-S-H-E-F-T-F-C-V-D-D

(SEQ ID NO:138)

P-G-W-C-Y-S-D-I-W-G-F-K-H-F-C-N-L-D

(SEQ ID NO:139)

D-S-S-C-I-K-H-H-N-K-V-T-C-F-F-P

(SEQ ID NO:140)

R-W-S-C-W-G-V-W-G-C-V-W-V

(SEQ ID NO:141)

P-V-D-C-K-H-H-F-W-W-C-Y-W-N

(SEQ ID NO:142)

S-W-N-C-A-F-H-H-N-E-M-V-W-C-D-D-G

(SEQ ID NO:143)

Y-W-Y-C-W-F-P-D-R-P-E-C-P-L-Y

(SEQ ID NO:186)

N-P-M-C-W-R-A-S-W-W-E-D-A-Y-C-I-N-H

(SEQ ID NO:187)

N-P-M-C-W-R-A-H-W-W-E-D-A-Y-C-I-N-H

(SEQ ID NO:188)

E-H-M-C-V-Y-T-T-W-G-E-L-I-W-C-D-N-H

(SEQ ID NO:189)

A-C-V-Y-T-T-W-G-E-L-I-W-C-D-N-H

(SEQ ID NO:190)

T-C-V-Y-T-T-W-G-E-L-I-W-C-D-N-H

(SEQ ID NO:191)

E-C-V-Y-T-T-W-G-E-L-I-W-C-D-N-H

(SEQ ID NO:192)

V-C-V-Y-T-T-W-G-E-L-I-W-C-D-N-H

(SEQ ID NO:193)

[Nle]-C-V-Y-T-T-W-G-E-L-I-W-C-D-N-H

(SEQ ID NO:194)

S-R-A-C-S-R-D-W-S-G-A-L-V-W-C-A-G-H

(SEQ ID NO:195)

E-R-A-C-S-R-D-W-S-G-A-L-V-W-C-A-G-H

(SEQ ID NO:196)

A-C-S-R-D-W-S-G-A-L-V-W-C-A-G-H

(SEQ ID NO:197)

T-C-S-R-D-W-S-G-A-L-V-W-C-A-G-H

(SEQ ID NO:198)

E-C-S-R-D-W-S-G-A-L-V-W-C-A-G-H

(SEQ ID NO:199)

V-C-S-R-D-W-S-G-A-L-V-W-C-A-G-H and

(SEQ ID NO:200)

G-C-S-R-D-W-S-G-A-L-V-W-C-A-G-H

N-terminal and/or C-terminal truncations of such Fc-region binding polypeptides down to the cyclic polypeptide, that retain binding affinity for antibody Fc-regions are also contemplated.

In addition, it is also envisioned that the phage display method of the current invention can be used to isolate additional families of binding molecules specific for Fc-region amino acid sequences.

Preferred Fc-region binding molecules according to the above formulae will include the following:

polypeptides of formula I, in which X₁ is G; X₂ is A or E; X₃ is L; and X₄ is I or V;

polypeptides of formula II, in which X is F or Y; and in the tripeptide of formula IIA, X₁ is C or Y;

X₂ is C, K, N or T; and X₃ is F, I, K, Q or V.

The most preferred binding molecules for isolation or detection of Fc-region amino acid sequences, as well as larger molecules that include these sequences, in a solution include the following polypeptides:

| | |
|---|---|
| RRACSRDWSGALVWCAGH; | (SEQ ID NO:14) |
| DHMCVYTTWGELIWCDNH; | (SEQ ID NO:36) |
| KYWCSFWGLQCKT; | (SEQ ID NO:88) |
| PVDCKHHFWWCYWN; | (SEQ ID NO:141) |
| DDHCYWFREWFNSECPHG; | (SEQ ID NO:50) |
| YYWCNYWGLCPDQ; | (SEQ ID NO:56) |
| PHNCDDHYWYCKWF; | (SEQ ID NO:115) |
| SYWCKIWDVCPQS; | (SEQ ID NO:57) |
| KYWCNLWGVCPAN; | (SEQ ID NO:58) |
| AATCSTSYWYYQWFCTDS; | (SEQ ID NO:124) |
| TYWCTFWELPCDPA; | (SEQ ID NO:108) |
| YWYCWFPDRPECPLY; | (SEQ ID NO:143) |
| SWVCWKAKWWEDKRCAPF; | (SEQ ID NO:130) |
| NPMCWKKSWWEDAYCINH; and | (SEQ ID NO:129) |
| SWNCAFHHEMVWCDDG. | (SEQ ID NO:142) |

Particularly preferred polypeptides, having amino-terminal and carboxy-terminal modifications to facilitate labeling or immobilization, include:

| | |
|---|---|
| Ac-GDDHMCVYTTWGELIWCDNHEPGPEGGGK-NH₂ ; | (SEQ ID NO:144, designated DX249) |
| Ac-AGKYWCSFWGLQCKTGTPGPEGGGK-NH₂ ; | (SEQ ID NO:146, designated DX250) |
| Ac-AGPVDCKHHFWWCYWNGTPGPEGGGK-NH₂ ; | (SEQ ID NO:153, designated DX251) |
| Ac-GDDDHCYWFREWFNSECPHGEPGPEGGGK-NH₂ ; | (SEQ ID NO:154, designated DX252) |
| Ac-GDRRACSRDWSGALVWCAGHEPGPEGGGK-NH2₂ ; | (SEQ ID NO:145, designated DX253) |
| Ac-AGYYWCNYWGLCPDQGTPGPEGGGK-NH₂ ; | (SEQ ID NO:155, designated DX254) |
| Ac-AGPHNCDDHYWYCKWFPGPEGGGK-NH₂ ; | (SEQ ID NO:150, designated DX389) |
| Ac-AGSYWCKIWDVCPQSPGPEGGGK-NH₂ ; | (SEQ ID NO:147, designated DX392) |
| Ac-AGKYWCNLWGVCPANPGPEGGGK-NH₂ ; | (SEQ ID NO:148, designated DX395) |
| Ac-AGAATCSTSYWYYQWFCTDSPGPEGGGK-NH₂ ; | (SEQ ID NO:151, designated DX398) |
| Ac-AGTYWCTFWELPCDPAPGPEGGGK-NH₂ ; | (SEQ ID NO:149, designated DX404) |
| Ac-AGYWYCWFPDRPECPLYPGPEGGGK-NH₂ ; | (SEQ ID NO:152, designated DX413) |
| Ac-GDSWVCWKAKWWEDKRCAPFGTPGPEGGGK-NH₂ ; | (SEQ ID NO:156, designated DX595) |
| Ac-GDNPMCWKKSWWEDAYCINHGTPGPEGGGK-NH₂ ; | (SEQ ID NO:157, designated DX596) |
| Ac-GDSWNCAFHHNEMVWCDDGGTPGPEGGGK-NH₂ ; | (SEQ ID NO:158, designated DX597) |
| Ac-GDWGECTVTSYGELIWCGGLEPGPEGGGK-NH₂ ; | (SEQ ID NO:159, designated DX1070) |
| Ac-GDNPMCWRASWWEDAYCINHEPGPEGGGK-NH₂ ; | (SEQ ID NO:160, designated DX1071) |
| Ac-GDNPMCWRASWWEDAYCINHEPGPEGGGK-NH₂ ; | (SEQ ID NO:161, designated DX1072) |
| Ac-GDDHMCVYTTWGELIWCDNHEPGPEG-X-NH₂ ; | (SEQ ID NO:162, designated DX877) |
| Ac-GDDHMCVYTTWGELIWCDNHEPG-X-Su-X-NH₂ ; | (SEQ ID NO:163, designated DX878) |
| Ac-GDDHMCVYTTWGELIWCDNHEPG-X-Z-X-NH₂ ; | (SEQ ID NO:164, designated DX905) |
| Ac-GDDHMCVYTTWGELIWCDNH-X-NH₂ ; | (SEQ ID NO:165, designated DX907) |
| Ac-GDDHMCVYTTWGELIWCDNH-X-Su-X-NH₂ ; | (SEQ ID NO:166, designated DX909) |
| Ac-GDDHMCVYTTWGELIWCDNH-X-Z-X-NH₂ ; | (SEQ ID NO:167, designated DX911) |
| Ac-DHMCVYTTWGELIWCDNHEPGPEGGGK-NH₂ ; | (SEQ ID NO:169, designated DX1062) |
| Ac-EHMCVYTTWGELIWCDNHEPGPEGGGK-NH₂ ; | (SEQ ID NO:170, designated DX1063) |
| Ac-ACVYTTWGELIWCDNHEPGPEGGGK-NH₂ ; | (SEQ ID NO:171, designated DX1064) |
| Ac-TCVYTTWGELIWCDNHEPGPEGGGK-NH₂ ; | (SEQ ID NO:172, designated DX1065) |
| Ac-ECVYTTWGELIWCDNHEPGPEGGGK-NH₂ ; | (SEQ ID NO:173, designated DX1066) |
| Ac-VCVYTTWGELIWCDNHEPGPEGGGK-NH₂ ; | (SEQ ID NO:174, designated DX1067) |
| Ac-[Nle]CVYTTWGELIWCDNHEPGPEGGGK-NH₂ ; | (SEQ ID NO:175, designated DX1068) |
| Ac-CVYTTWGELIWCDNHEPGPEGGGK-NH₂ ; | (SEQ ID NO:176, designated DX1069) |
| Ac-SRACSRDWSGALVWCAGHEPGPEGGGK-NH₂ ; | (SEQ ID NO:177, designated DX1139) |
| Ac-RRACSRDWSGALVWCAGHEPGPEGGGK-NH₂ ; | (SEQ ID NO:178, designated DX1142) |
| Ac-ERACSRDWSGALVWCAGHEPGPEGGGK-NH₂ ; | (SEQ ID NO:179, designated DX1141) |
| Ac-ACSRDWSGALVWCAGHEPGPEGGGK-NH₂ ; | (SEQ ID NO:180, designated DX1142) |
| Ac-TCSRDWSGALVWCAGHEPGPEGGGK-NH₂ ; | (SEQ ID NO:181, designated DX1143) |
| Ac-ECSRDWSGALVWCAGHEPGPEGGGK-NH₂ ; | (SEQ ID NO:182, designated DX1144) |
| Ac-VCSRDWSGALVWCAGHEPGPEGGGK-NH₂ ; | (SEQ ID NO:183, designated DX1145) |
| Ac-GCSRDWSGALVWCAGHEPGPEGGGK-NH₂ ; and | (SEQ ID NO:184, designated DX1146) |
| Ac-CSRDWSGALVWCAGHEPGPEGGGK-NH₂ . | (SEQ ID NO:185, designated DX1147) |

In the foregoing polypeptides, Ac-denotes N-terminal acetylation, —NH₂ denotes C-terminal amidation, —X—NH₂ dentoes the C-terminal group —NH—(CH₂CH₂O)₂—CH₂CH₂—NH₂, —X—Su—X—NH₂ denotes the C-terminal group —NH—(CH₂CH₂O)₂—CH₂CH₂—NH—C:O—CH₂CH₂—C:O—NH—(CH₂CH₂O)₂—CH₂CH₂—NH₂, —X—Z—X—NH₂ denotes the C-terminal group —NH—(CH₂CH₂O)₂—CH₂CH₂—NH—C:O—CH₂—O—(CH₂CH₂O)₂—CH₂—C:O—NH—CH₂CH₂O)₂—CH₂CH₂—NH₂, and [Nle] denotes norleucine. For binding studies described herein, certain of these polypeptides are fluorescein-labeled, the fluorophore being attached at the ε-amino group of the carboxy-terrninal lysine. In fluoresceinated form, the polypeptide DX249 (i.e., Ac-GDDHMCVYTTWGELIWCDNHEPGPEGGGK-NH₂, SEQ ID NO:144) is designated DX276; in fluoresceinated form, the polypeptide DX250 (i.e., Ac-AGKYWCSFWGLQCKTGTPGPEGGGK-NH₂, SEQ ID NO:146) is designated DX300; in fluoresceinated form, the polypeptide DX253 (i.e., Ac-GDRRACSRDWSGALVWCAGHEPGPEGGGK-NH₂, SEQ ID NO:145) is designated DX301.

The polypeptides of the invention show specific binding to Fc-region polypeptides. Preferred polypeptides disclosed herein show high affinity (e.g., K_{D} in the range 10 μM to 0.01 μM, more preferably in the range 1.0 μM to 0.01 μM) for human Fc polypeptides or particular IgG isotypes (e.g., IgG1, IgG2, IgG3 and/or IgG4). Some polypeptides also show species specificity (e.g., binding to human but not other mammalian IgGs). For example:

DX249, exhibits dissociation constants (K_{D})for human IgG1 of less than 0.1 μM at pH 5.7 and less than 0.5 μM at pH 7.4 (see Table 1, infra);

DX252, exhibits dissociation constants (K_{D})for human IgG3 of less than 0.1 μM at pH 5.7 and in the range of ˜2.1 μM to ˜3.4 μM for IgG1, IgG2, IgG3, and IgG4 at pH 7.4 (see Table 1, infra);

DX253, exhibits quantitative binding of Fc protein (capture efficiency >90% of total load) from buffer solution and tobacco extract (see Examples 7 and 8, infra);

DX254, exhibits dissociation constants (K_{D}) for human IgG1 of less than 0.1 μM at pH 5.7, less than 2.0 μM at pH 7.4, and less than 1.0 μM at pH 9.3 (see Table 1, infra);

DX301, exhibits dissociation constants below about 10 μM for human Fc, IgG1, IgG2 and IgG4 (see Table 8, infra); and

DX300, exhibits a dissociation constant of 4.1±4.6 for human IgG3 (see Table 6).

The present invention also discloses a method for detecting a polypeptide target which comprises at least one immunoglobulin Fc region amino acid sequence in a solution suspected of containing it comprising:

(a) contacting said solution with a polypeptide according to claim 1 or claim 5; and

(b) determining whether binding has occurred between said polypeptide and said polypeptide target.

In a further embodiment, the invention relates to a method for isolating a polypeptide target which comprises at least one immunoglobulin Fc region amino acid sequence in a solution containing said polypeptide target, said method comprising:

(a) preparing a Fc binding polypeptide according to the invention by immobilizing the Fc binding polypeptide on a solid support or conjugating the binding polypeptide with an affinity tag;

(b) contacting the solution containing said polypeptide target with the binding polypeptide of step (a); and

(c) separating the non-binding components of said solution from the binding polypeptide of step (b).

The present invention also contemplates eluting the bound Fc-containing peptide from the solid support.

The present invention also contemplates a solid-phase detection assay for target polypeptides containing Fc-region polypeptides, comprising the steps:

(a) contacting a solution suspected of containing a target polypeptide comprising an Fc-region polypeptide with a binding molecule for the target polypeptide, which binding molecule is immobilized on a solid support,

(b) separating the target bound to the solid support from the unbound components of the solution,

(c) contacting the solid support with an Fc binding polypeptide according to the invention which has been detectably labeled, and

(d) detecting binding of the labeled Fc binding polypeptide to said solid support.

In the foregoing methods, the solution in which Fc-region targets are isolated or detected may be selected from the group consisting of whole blood, plasma, transgenic milk, eggs of transgenic birds, and conditioned media.

Also, in the foregoing methods, the polypeptide target can be an antibody or an antibody fragment containing all or part of an Fc region. Preferably, in the foregoing methods, said polypeptide target is an antibody.

Where the target polypeptide is an antibody, it can be a human IgG, and may be of any particular isotype, e.g., human IgG1, IgG2, IgG3, IgG4, or combinations thereof.

In the foregoing methods, the solid support may be, for example, cellulose, controlled-pore glass, silica, polystyrene, styrene divinyl benzene, agarose, and crosslinked agarose.

The present invention also relates to recombinant bacteriophage, or “phage” (including “phagemid”) expressing an exogenous polypeptide capable of binding to an immunoglobulin Fc region, said exogenous polypeptide comprising an amino acid sequence of any of the formulae I, II, III or IV:

Z₁—X₁—X₂—X₃—X₄—W—C—Z₂(SEQ ID NO:4); I.

wherein,

Z₁ is a polypeptide of at least 6 amino acids;

X₁ is G, H, N, R, or S;

X₂ is A, D, E, F, I, M, or S;

X₃ is A, I, L, M, or V;

X₄ is I, M, T, or V;

Z₂ is a polypeptide of at least one amino acid or is absent; and

Z₁ contains at least one cysteine residue such that formation of a disulfide bond with the invariant cysteine residue forms a cyclic peptide of 12 amino acids;

Z₁—X—W—Z₂—W—Z₃(SEQ ID NO:5) II.

wherein,

Z₁ is a polypeptide of at least one amino acid or is absent;

X is F or Y;

Z₂ is a tripeptide; and

Z₃ is a polypeptide of at least one amino acid; and

wherein at least two of the polypeptides Z₁, Z₂, and Z₃ contain a cysteine residue, such that formation of a disulfide bond between such cysteine residues forms a cyclic peptide of 7-12 amino acids;

Z₁—W—Z₂—W—W—Z₃(SEQ ID NO:6); III.

wherein,

Z₁ is a polypeptide of at least one amino acid;

Z₂ is a tripeptide; and

Z₃ is a polypeptide of at least one amino acid;

wherein at least two of the polypeptides Z₁, Z₂, and Z₃ contain a cysteine residue, such that formation of a disulfide bond between such cysteine residues forms a cyclic peptide of 8-12 amino acids, with the proviso that where Z₁ contains a cysteine, then Z₂ does not contain a cysteine, and where Z₂ contains a cysteine, it is the middle residue of the tripeptide and Z₃ also contains a cysteine;

Z₁—P—X₁—W—X₂—C—X₃—X₄—X₅(SEQ ID NO:7); IV.

wherein,

Z₁ is a polypeptide of at least one amino acid and includes a cysteine residue;

X₁ is A, E, R, S, or T;

X₂ is F, W, or Y;

X₃ is D, E, L, M, or Q;

X₄ is H, W, or Y;

X₅ is F or Y; and

wherein the cysteine residue in Z₁ and the cysteine residue between X₂ and X₃ form a cyclic peptide of 10-12 amino acids.

Preferably, in the foregoing recombinant bacteriophage according to the invention, said exogenous polypeptide comprises an amino acid sequence of the formula:

Z₁—X₁—X₂—X₃—X₄—W—C—Z₂(SEQ ID NO:4);

wherein,

X₁ is G;

X₂ is A or E;

X₃ is L;

X₄ is I or V; or

said exogenous polypeptide comprises an amino acid sequence of the formula:

Z₁—X—W—Z₂—W—Z₃(SEQ ID NO:5)

wherein,

X is F or Y; and

wherein Z₂ is a peptide of the formula:

X₁—X₂—X₃,

wherein,

X₁ is C or Y;

X₂ is C, K, N, or T, with the proviso that X₂ is not C if X₁ is C, and

X₃ is F, I, K, Q, or V.

More preferably, the recombinant bacteriophage according to the invention will display an exogenous polypeptide which comprises an amino acid sequence selected from the group consisting of:

(SEQ ID NO:14)

R-R-A-C-S-R-D-W-S-G-A-L-V-W-C-A-G-H

(SEQ ID NO:15)

W-G-E-C-T-V-T-S-Y-G-E-L-I-W-C-G-G-L

(SEQ ID NO:16)

S-S-A-C-A-F-D-P-M-G-A-V-I-W-C-T-Y-D

(SEQ ID NO:17)

L-L-E-C-A-Y-N-T-S-G-E-L-I-W-C-N-G-S

(SEQ ID NO:18)

P-D-D-C-S-I-H-F-S-G-E-L-I-W-C-E-P-L

(SEQ ID NO:19)

L-G-E-C-T-V-T-S-Y-G-E-L-I-W-C-G-G-L

(SEQ ID NO:20)

W-G-E-C-T-V-T-S-Y-G-E-L-I-W-C-G-G-H

(SEQ ID NO:21)

D-H-M-C-V-Y-T-T-W-G-E-L-I-W-C-D-D-H

(SEQ ID NO:22)

W-G-E-C-T-V-T-S-Y-G-E-L-I-W-C-G-G-L

(SEQ ID NO:23)

C-R-A-C-S-R-D-W-P-G-A-L-V-W-C-A-G-H

(SEQ ID NO:24)

R-R-A-C-S-R-D-W-S-G-A-L-V-W-C-A-G-H

(SEQ ID NO:25)

L-H-A-C-A-F-D-P-M-G-A-V-I-W-C-T-Y-D

(SEQ ID NO:26)

D-H-M-C-V-Y-T-T-W-G-E-L-M-W-C-D-N-H

(SEQ ID NO:27)

P-P-T-C-T-W-D-W-Q-G-I-L-V-W-C-S-G-H

(SEQ ID NO:28)

S-N-K-C-S-N-T-W-D-G-S-L-I-W-C-S-A-N

(SEQ ID NO:29)

F-P-E-C-T-F-D-M-E-G-F-L-I-W-C-S-S-F

(SEQ ID NO:30)

H-D-L-C-A-Q-A-P-F-G-D-A-T-W-C-D-L-R

(SEQ ID NO:31)

P-N-H-C-S-Y-N-L-K-S-E-L-I-W-C-Q-D-L

(SEQ ID NO:32)

P-L-D-C-A-R-D-I-H-N-S-L-I-W-C-S-L-G

(SEQ ID NO:33)

G-S-E-C-S-W-T-S-L-N-E-L-I-W-C-A-H-W

(SEQ ID NO:34)

W-P-D-C-S-F-T-V-Q-R-D-L-I-W-C-E-A-L

(SEQ ID NO:35)

S-H-S-C-A-Y-D-Y-A-H-M-L-V-W-C-T-H-F

(SEQ ID NO:36)

D-H-M-C-V-Y-T-T-W-G-E-L-I-W-G-D-N-H

(SEQ ID NO:37)

R-P-N-C-T-F-A-A-S-G-E-L-I-W-C-M-H-Y

(SEQ ID NO:38)

W-W-G-C-Q-F-D-W-R-G-E-L-V-W-C-P-Y-L

(SEQ ID NO:39)

G-G-V-C-S-Y-S-G-M-G-E-I-V-W-C-R-W-F

(SEQ ID NO:40)

A-L-M-C-S-H-D-M-W-G-S-L-I-W-C-K-H-F

(SEQ ID NO:41)

W-W-N-C-H-N-G-W-T-W-T-G-G-W-C-W-W-F

(SEQ ID NO:42)

Y-H-V-C-A-R-D-S-W-D-Q-L-I-W-C-E-A-F

(SEQ ID NO:43)

N-Y-W-C-N-F-W-Q-L-P-T-C-D-N-L

(SEQ ID NO:44)

Y-W-Y-C-K-W-F-S-E-S-A-S-C-S-S-R

(SEQ ID NO:45)

Y-W-Y-C-K-W-F-E-D-K-H-P-C-D-S-S

(SEQ ID NO:46)

Y-W-Y-C-S-W-F-P-D-R-P-D-C-P-L-Y

(SEQ ID NO:47)

N-Y-W-C-N-V-W-L-L-G-D-V-C-R-S-H

(SEQ ID NO:48)

L-Y-W-C-H-V-W-F-G-Q-H-A-W-Q-C-K-Y-P

(SEQ ID NO:49)

Y-W-K-C-K-W-M-P-W-M-C-G-F-D

(SEQ ID NO:50)

D-D-H-C-Y-W-F-R-E-W-F-N-S-E-C-P-H-G

(SEQ ID NO:51)

N-Y-W-C-N-I-W-G-L-H-G-C-N-S-H

(SEQ ID NO:52)

Y-W-F-C-Q-W-F-S-Q-N-H-T-C-F-R-D

(SEQ ID NO:53)

H-Y-W-C-D-I-W-F-G-A-P-A-C-Q-F-R

(SEQ ID NO:54)

S-G-D-C-G-F-W-P-R-I-W-G-L-C-M-D-N

(SEQ ID NO:55)

F-W-Y-C-K-W-F-Y-E-D-A-Q-C-S-H-D

(SEQ ID NO:56)

Y-Y-W-C-N-Y-W-G-L-C-P-D-Q

(SEQ ID NO:57)

S-Y-W-C-K-I-W-D-V-C-P-Q-S

(SEQ ID NO:58)

K-Y-W-C-N-L-W-G-V-C-P-A-N

(SEQ ID NO:59)

Q-Y-W-C-Y-Q-W-G-L-C-G-A-N

(SEQ ID NO:60)

K-Y-W-C-Q-Q-W-G-V-C-N-G-S

(SEQ ID NO:61)

K-Y-W-C-V-Q-W-G-V-C-P-E-S

(SEQ ID NO:62)

K-Y-W-C-M-Q-W-G-L-C-G-W-E

(SEQ ID NO:63)

H-F-W-C-E-V-W-G-L-C-P-S-I

(SEQ ID NO:64)

Q-Y-W-C-T-K-W-G-L-C-T-N-V

(SEQ ID NO:65)

A-Y-W-C-K-V-W-G-L-C-Q-G-E

(SEQ ID NO:66)

K-Y-W-C-N-L-W-G-V-C-P-A-N

(SEQ ID NO:67)

Q-Y-W-C-N-V-W-G-V-C-L-P-S

(SEQ ID NO:68)

H-Y-W-C-Q-Q-W-G-I-C-E-R-P

(SEQ ID NO:69)

R-Y-W-C-N-I-W-D-V-C-P-E-Q

(SEQ ID NO:70)

Q-Y-W-C-T-H-W-G-L-C-G-K-Y

(SEQ ID NO:71)

T-Y-W-C-T-K-W-G-L-C-P-H-N

(SEQ ID NO:72)

F-Y-W-C-G-Q-W-G-L-C-A-P-P

(SEQ ID NO:73)

G-Y-W-C-N-V-W-G-L-C-S-T-E

(SEQ ID NO:74)

R-Y-W-C-G-V-W-G-V-C-E-I-D

(SEQ ID NO:75)

K-F-W-C-T-I-W-G-V-C-H-M-P

(SEQ ID NO:76)

H-Y-W-C-Q-Q-W-G-I-C-E-R-P

(SEQ ID NO:77)

R-Y-W-C-N-I-W-D-V-C-P-E-Q

(SEQ ID NO:78)

F-Y-W-C-S-Q-W-G-L-C-K-Y-D

(SEQ ID NO:79)

H-Y-W-C-E-K-W-G-L-C-L-M-S

(SEQ ID NO:80)

H-Y-W-C-Q-K-W-G-V-C-P-T-D

(SEQ ID NO:81)

H-Y-W-C-S-L-W-G-V-C-D-I-N

(SEQ ID NO:82)

R-F-W-C-S-A-W-G-V-C-P-A

(SEQ ID NO:83)

S-Y-W-C-K-I-W-D-V-C-P-Q-S

(SEQ ID NO:84)

Q-Y-W-C-S-I-W-K-V-C-P-G-R

(SEQ ID NO:85)

Y-W-Y-C-E-W-F-G-A-C-I-N-D

(SEQ ID NO:86)

E-Y-W-C-K-Y-W-G-L-E-C-V-H-R

(SEQ ID NO:87)

K-Y-W-C-T-Q-W-G-L-K-C-D-K-Q

(SEQ ID NO:88)

K-Y-W-C-S-F-W-G-L-Q-C-K-T

(SEQ ID NO:89)

R-Y-W-C-N-F-W-G-V-N-C-D-A-N

(SEQ ID NO:90)

N-Y-W-C-T-H-W-G-V-M-C-L-D-H

(SEQ ID NO:91)

Y-W-F-C-K-W-F-P-S-Q-C-Q-F-M

(SEQ ID NO:92)

A-Y-W-C-K-Q-W-G-L-K-C-Q-L-G

(SEQ ID NO:93)

K-Y-W-C-K-F-W-G-L-E-C-K-V-G

(SEQ ID NO:94)

N-Y-W-C-T-E-W-G-L-N-C-N-N-K

(SEQ ID NO:95)

S-Y-W-C-E-K-W-G-L-T-C-E-T-H

(SEQ ID NO:96)

E-Y-W-C-R-I-W-G-L-Q-C-N-M-V

(SEQ ID NO:97)

K-Y-W-C-K-K-W-G-V-N-C-D-F-N

(SEQ ID NO:98)

K-Y-W-C-S-V-W-G-V-Q-C-P-H-S

(SEQ ID NO:99)

F-Y-W-C-T-K-W-G-L-E-C-I-H-S

(SEQ ID NO:100)

H-Y-W-C-Q-Q-W-G-L-M-C-F-E-T

(SEQ ID NO:1O1)

K-Y-W-C-K-R-W-G-L-M-C-N-G-G

(SEQ ID NO:102)

A-Y-W-C-M-T-W-G-V-P-C-I-S-W

(SEQ ID NO:103)

K-Y-W-C-K-K-W-G-V-N-C-D-F-N

(SEQ ID NO:104)

K-Y-W-C-S-V-W-G-V-Q-C-P-D-S

(SEQ ID NO:105)

K-Y-W-C-S-V-W-G-V-Q-C-P-H-S

(SEQ ID NO:106)

L-Y-W-C-T-K-W-G-V-T-C-Q-K-D

(SEQ ID NO:107)

T-Y-W-C-H-K-W-G-V-K-C-A-T-T

(SEQ ID NO:108)

T-Y-W-C-T-F-W-E-L-P-C-D-P-A

(SEQ ID NO:109)

K-Y-W-C-T-K-W-Q-L-N-C-E-E-V

(SEQ ID NO:110)

N-Y-W-C-H-F-W-Q-V-P-C-L-E-Q

(SEQ ID NO:111)

T-Y-W-C-V-V-W-N-V-P-C-S-T-D

(SEQ ID NO:112)

N-F-W-C-H-T-W-G-L-Q-C-N-D-L

(SEQ ID NO:113)

F-W-Y-C-Y-W-F-N-E-K-C-K-T-P

(SEQ ID NO:114)

G-F-W-C-T-F-W-G-V-T-C-E-A-G

(SEQ ID NO:115)

P-H-N-C-D-D-H-Y-W-Y-C-K-W-F

(SEQ ID NO:116)

E-M-T-C-S-S-H-Y-W-Y-C-T-W-M

(SEQ ID NO:117)

H-I-D-C-K-T-N-Y-W-W-C-R-W-T

(SEQ ID NO:118)

E-M-R-C-G-Q-H-F-W-Y-C-E-W-F

(SEQ ID NO:119)

N-Y-W-C-N-F-W-Q-L-P-T-C-D-N-L

(SEQ ID NO:120)

Y-W-Y-C-Q-W-F-Q-E-V-N-K-C-F-N-S

(SEQ ID NO:121)

Y-Y-W-C-R-H-W-F-P-D-F-D-C-V-H-S

(SEQ ID NO:122)

Y-W-Y-C-S-W-F-P-D-R-P-D-C-P-L-Y

(SEQ ID NO:123)

Y-W-Y-C-V-W-F-D-N-A-D-Q-C-V-H-H

(SEQ ID NO:124)

A-A-T-C-S-T-S-Y-W-Y-Y-Q-W-F-C-T-D-S

(SEQ ID NO:125)

Y-W-A-C-V-W-G-L-K-S-C-V-D-R

(SEQ ID NO:126)

Y-W-R-C-V-W-F-P-A-S-C-P-T

(SEQ ID NO:127)

D-W-Q-C-L-W-W-G-N-S-F-W-P-Y-C-A-N-L

(SEQ ID NO:128)

F-W-R-C-H-W-W-P-E-R-C-P-V-D

(SEQ ID NO:129)

N-P-M-C-W-K-K-S-W-W-E-D-A-Y-C-I-N-H

(SEQ ID NO:130)

S-W-V-C-W-K-A-K-W-W-E-D-K-R-C-A-P-F

(SEQ ID NO:131)

S-R-Q-C-W-K-E-L-W-W-T-D-Q-M-C-L-D-L

(SEQ ID NO:132)

S-F-R-C-Q-S-S-F-P-S-W-Y-C-D-Y-Y

(SEQ ID NO:133)

S-W-H-C-Q-N-T-Y-P-E-W-Y-C-Q-W-Y

(SEQ ID NO:134)

G-S-K-C-K-Q-T-G-F-P-R-W-W-C-E-H-Y

(SEQ ID NO:135)

D-G-V-C-G-P-R-G-F-G-P-A-W-F-C-M-H-Y

(SEQ ID NO:136)

Y-S-H-C-A-T-H-Y-P-T-W-Y-C-L-H-F

(SEQ ID NO:137)

F-C-N-C-W-G-S-H-E-F-T-F-C-V-D-D

(SEQ ID NO:138)

P-G-W-C-Y-S-D-I-W-G-F-K-H-F-C-N-L-D

(SEQ ID NO:139)

D-S-S-C-I-K-H-H-N-K-V-T-C-F-F-P

(SEQ ID NO:140)

R-W-S-C-W-G-V-W-G-C-V-W-V

(SEQ ID NO:141)

P-V-D-C-K-H-H-F-W-W-C-Y-W-N

(SEQ ID NO:142)

S-W-N-C-A-F-H-H-N-E-M-V-W-C-D-D-G

(SEQ ID NO:143)

Y-W-Y-C-W-F-P-D-R-P-E-C-P-L-Y

(SEQ ID NO:186)

N-P-M-C-W-R-A-S-W-W-E-D-A-Y-C-I-N-H

(SEQ ID NO:187)

N-P-M-C-W-R-A-H-W-W-E-D-A-Y-C-I-N-H

(SEQ ID NO:188)

E-H-M-C-V-Y-T-T-W-G-E-L-I-W-C-D-N-H

(SEQ ID NO:189)

A-C-V-Y-T-T-W-G-E-L-I-W-C-D-N-H

(SEQ ID NO:190)

T-C-V-Y-T-T-W-G-E-L-I-W-C-D-N-H

(SEQ ID NO:191)

E-C-V-Y-T-T-W-G-E-L-I-W-C-D-N-H

(SEQ ID NO:192)

V-C-V-Y-T-T-W-G-E-L-I-W-C-D-N-H

(SEQ ID NO:194)

S-R-A-C-S-R-D-W-S-G-A-L-V-W-C-A-G-H

(SEQ ID NO:195)

E-R-A-C-S-R-D-W-S-G-A-L-V-W-C-A-G-H

(SEQ ID NO:196)

A-C-S-R-D-W-S-G-A-L-V-W-C-A-G-H

(SEQ ID NO:197)

T-C-S-R-D-W-S-G-A-L-V-W-C-A-G-H

(SEQ ID NO:198)

E-C-S-R-D-W-S-G-A-L-V-W-C-A-G-H

(SEQ ID NO:199)

V-C-S-R-D-W-S-G-A-L-V-W-C-A-G-H and

(SEQ ID NO:200)

G-C-S-R-D-W-S-G-A-L-V-W-C-A-G-H.

The present invention further relates to a method for detecting a polypeptide target comprising at least one immunoglobulin Fc region amino acid sequence in a solution, comprising:

(a) contacting said solution with a bacteriophage according to the present invention; and

(b) determining whether binding has occurred between said polypeptide target and said bacteriophage.

In the foregoing method, said solution may be selected from the group consisting of whole blood, plasma, transgenic milk, eggs of transgenic birds, and conditioned media. Also, said polypeptide target can be an antibody or antibody fragment containing all or part of an Fc region. Preferably, said polypeptide target is an antibody. Where the target is an antibody, it is preferably a human IgG, and may be selected from the group consisting of human IgG1, IgG2, IgG3, IgG4, and combinations thereof.

In the foregoing method of using a bacteriophage according to the invention, said bacteriophage is preferably a phage or phagemid.

In a further embodiment, the invention relates to separation media comprising:

(a) a chromatographic matrix material, and, immobilized thereon;

(b) an Fc binding polypeptide according to the invention, e.g., SEQ ID NOs: 4-200.

In preferred embodiments, said chromatographic matrix material is selected from the group consisting of cellulose, silica gel-type resins or membranes, crosslinked polysaccharides, and agarose. In preferred embodiments, said chromatographic matrix material is an amine-reactive chromatographic matrix material, preferably an aldehyde-functional methacrylate resin, a formyl methacrylate resin, or, most preferably, an NHS-activated agarose resin.

In preferred embodiments, the separation media of this invention comprise the reaction product of:

(a) an amine-reactive chromatographic matrix material; and

(b) a polypeptide selected from the group consisting of:

| | |
|---|---|
| Ac-GDDHMCVYTTWGELIWCDNHEPGPEGGGK-NH₂ ; | (SEQ ID NO:144) |
| Ac-AGKYWCSFWGLQCKTGTPGPEGGGK-NH₂ ; | (SEQ ID NO:146) |
| Ac-AGPVDCKHHFWWCYWNGTPGPEGGGK-NH₂ ; | (SEQ ID NO:153) |
| Ac-GDDDHCYWFREWFNSECPHGEPGPEGGGK-NH₂ ; | (SEQ ID NO:154) |
| Ac-GDRRACSRDWSGALVWCAGHEPGPEGGGK-NH₂ ; | (SEQ ID NO:145) |
| Ac-AGYYWCNYWGLCPDQGTPGPEGGGK-NH₂ ; | (SEQ ID NO:155) |
| Ac-AGPHNCDDHYWYCKWFPGPEGGGK-NH₂ ; | (SEQ ID NO:150) |
| Ac-AGSYWCKIWDVCPQSPGPEGGGK-NH₂ ; | (SEQ ID NO:147) |
| Ac-AGKYWCNLWGVCPANPGPEGGGK-NH₂ ; | (SEQ ID NO:148) |
| Ac-AGAATCSTSYWYYQWFCTDSPGPEGGGK-NH₂ ; | (SEQ ID NO:151) |
| Ac-AGTYWCTFWELPCDPAPGPEGGGK-NH₂ ; | (SEQ ID NO:149) |
| Ac-AGYWYCWFPDRPECPLYPGPEGGGK-NH₂ ; | (SEQ ID NO:152) |
| Ac-GDSWVCWKAKWWEDKRCAPFGTPGPEGGGK-NH₂ ; | (SEQ ID NO:156) |
| Ac-GDNPMCWKKSWWEDAYCINHGTPGPEGGGK-NH₂ ; | (SEQ ID NO:157) |
| Ac-GDSWNCAFHHNEMVWCDDGGTPGPEGGGK-NH₂ ; | (SEQ ID NO:158) |
| Ac-GDWGECTVTSYGELIWCGGLEPGPEGGGK-NH₂ ; | (SEQ ID NO:159) |
| Ac-GDNPMCWRASWWEDAYCINHEPGPEGGGK-NH₂ ; | (SEQ ID NO:160) |
| Ac-GDNPMCWRAHWWEDAYCINHEPGPEGGGK-NH₂ ; | (SEQ ID NO:161) |
| Ac-GDDHMCVYTTWGELIWCDNHEPGPEG-X-NH₂ ; | (SEQ ID NO:162) |
| Ac-GDDHMCVYTTWGELIWCDNHEPG-X-Su-X-NH₂ ; | (SEQ ID NO:163) |
| Ac-GDDHMCVYTTWGELIWCDNHEPG-X-Z-X-NH₂ ; | (SEQ ID NO:164) |
| Ac-GDDHMCVYTTWGELIWCDNH-X-NH₂ ; | (SEQ ID NO:165) |
| Ac-GDDHMCVYTTWGELIWCDNH-X-Su-X-NH₂ ; | (SEQ ID NO:166) |
| Ac-GDDHMCVYTTWGELIWCDNH-X-Z-X-NH₂ ; | (SEQ ID NO:167) |
| Ac-DHMCVYTTWGELIWCDNHEPGPEGGGK-NH₂ ; | (SEQ ID NO:169) |
| Ac-EHMCVYTTWGELIWCDNHEPGPEGGGK-NH₂ ; | (SEQ ID NO:170) |
| Ac-ACVYTTWGELIWCDNHEPGPEGGGK-NH₂ ; | (SEQ ID NO:171) |
| Ac-TCVYTTWGELIWCDNHEPGPEGGGK-NH₂ ; | (SEQ ID NO:172) |
| Ac-ECVYTTWGELIWCDNHEPGPEGGGK-NH₂ ; | (SEQ ID NO:173) |
| Ac-VCVYTTWGELIWCDNHEPGPEGGGK-NH₂ ; | (SEQ ID NO:174) |
| Ac-[Nle]CVYTTWGELIWCDNHEPGPEGGGK-NH₂ ; | (SEQ ID NO:175) |
| Ac-CVYTTWGELIWCDNHEPGPEGGGK-NH₂ ; | (SEQ ID NO:176) |
| Ac-SRACSRDWSGALVWCAGHEPGPEGGGK-NH₂ ; | (SEQ ID NO:177) |
| Ac-RRACSRDWSGALVWCAGHEPGPEGGGK-NH₂ ; | (SEQ ID NO:178) |
| Ac-ERACSRDWSGALVWCAGHEPGPEGGGK-NH₂ ; | (SEQ ID NO:179) |
| Ac-ACSRDWSGALVWCAGHEPGPEGGGK-NH₂ ; | (SEQ ID NO:180) |
| Ac-TCSRDWSGALVWCAGHEPGPEGGGK-NH₂ ; | (SEQ ID NO:181) |
| Ac-ECSRDWSGALVWCAGHEPGPEGGGK-NH₂ ; | (SEQ ID NO:182) |
| Ac-VCSRDWSGALVWCAGHEPGPEGGGK-NH₂ ; | (SEQ ID NO:183) |
| Ac-GCSRDWSGALVWCAGHEPGPEGGGK-NH₂ ; and | (SEQ ID NO:184) |
| Ac-CSRDWSGALVWCAGHEPGPEGGGK-NH₂ . | (SEQ ID NO:185) |

In the foregoing list, Ac-denotes N-terminal acetylation, —NH₂ denotes C-terminal amidation, [Nle] is norleucine, —X—NH₂ is —NH—(CH₂CH₂O)₂—CH₂CH₂—NH₂, —X—Su—X—NH₂ is —NH—(CH₂CH₂O)₂—CH₂CH₂—NH—C:O—CH₂CH₂—C:O—NH—(CH₂CH₂O)₂—CH₂CH₂—NH₂, and —X—Z—X—NH₂ is —NH—(CH₂CH₂O)₂—CH₂CH₂—NH—C:O—CH₂—O—(CH₂CH₂O)₂—CH₂—C:O—NH—(CH₂CH₂O)₂—CH₂CH₂—NH₂.

In the foregoing separation media, said matrix material is preferably an aldehyde-functional methacrylate chromatographic resin, a formyl-substituted ethylene glycol-methacrylate copolymer support, or an NHS-activated agarose support.

In another aspect, the present invention includes a method for separating a polypeptide target comprising at least one immnunoglobulin Fc region amino acid sequence from a solution containing it comprising:

(a) contacting said solution with separation media as defined above under binding conditions;

(b) removing unbound material; and

(c) eluting the bound polypeptide target from said separation media.

In the foregoing method, said polypeptide target can be an antibody or an antibody fragment containing all or part of an Fc region. Preferably, said polypeptide target is an antibody. Where the polypeptide is an antibody, it is preferably a human IgG, and may be selected from the group consisting of human IgG1, IgG2, IgG3, IgG4, and combinations thereof.

Also in the foregoing method, said solution may be whole blood, plasma, transgenic milk, eggs of transgenic birds, or conditioned media.

The present invention also contemplates replicable genetic packages displaying any of the foregoing Fc binding polypeptides (e.g., SEQ ID NOS:4-143 and 186-200). Such replicable genetic packages may be phage (including phagemids), bacteria, yeast, or any other suitable host cell.

Solutions from which Fc-region polypeptides may be isolated and purified from include, but are not limited to blood, blood fractions such a plasma, transgenic milk, eggs of transgenic chickens, conditioned media containing Fc-region polypeptides, i.e., any solution or feed stream that contains natural, recombinant or synthetic immunoglobulins, Fc-region-containing fragments thereof, Fc-region-containing fusion proteins or other Fc-region polypeptides.

In another aspect, the present invention provides a method for detecting an Fc-region polypeptide in a solution suspected of containing it, comprising contacting the solution with a binding molecule according to the invention and determining whether a binding complex has formed.

Fc-region polypeptides detected, isolated or purified according to the invention can be isolated Fc regions of an immunoglobulin, whole immunoglobulins, antibody fragments that include all or part of an Fc region, fusion proteins including an Fc region polypeptide, or polypeptides or other molecules conjugated with an Fc-region polypeptide bound by a binding molecule according to the invention.

In yet another aspect, the invention provides methods for increasing the serum half-life of a therapeutic or diagnostic compound of interest comprising linking the therapeutic or diagnostic compound to an Fc-binding polypeptide according to the present invention and administering the compound/Fc-binding polypeptide to an individual. The compound/binding moiety conjugate in the blood and will associate with circulating antibody molecules and will remain in the serum longer than if the compound were administered in the absence of an Fc-binding polypeptide. The Fc-binding polypeptide can be selected for its particular affinity for an antibody, immunoglobulin, or immunoglobulin subtype, so as to tailor the behavior of the conjugate in circulation to the particular therapeutic or diagnostic need for which the conjugate is employed.

In a preferred embodiment the present invention also discloses a method for increasing the serum half-life of a compound to be administered to an individual comprising:

(a) preparing a conjugate of said compound with an Fc binder polypeptide moiety according to the invention, wherein the conjugate has the ability to associate with circulating antibodies of the individual, and

(b) introducing said conjugate into the circulatory system of the individual.

In a preferred embodiment, said compound is a diagnostic compound and may include a radioactive label or a magnetic resonance imaging agent.

In yet another preferred embodiment, said compound is a therapeutic compound. For example, the compound may selected from thrombin inhibitors, thrombolytics, rennin inhibitors, ACE inhibitors, selectin ligands, inhibitors of the coagulation cascade, complement regulatory molecules, serine proteases, GPIIb/IIIa antagonists, and CRF antagonists.

These and other embodiments of the present invention are described herein in detail.

Definitions

As used herein, the term “recombinant” is used to describe non-naturally altered or manipulated nucleic acids, host cells transfected with exogenous nucleic acids, or polypeptides expressed non-naturally, through manipulation of isolated DNA and transformation of host cells. Recombinant is a term that specifically encompasses DNA molecules which have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides.

The term “bacteriophage” is defined as a bacterial virus containing a DNA core and a protective shell built up by the aggregation of a number of different protein molecules. The terms “bacteriophage” and “phage” are used herein interchangeably. Unless otherwise noted, the terms “bacteriophage” and “phage” also encompass “phagemids”, i.e., bacteriophage the genome of which includes a plasmid that can be excised by coinfection of a host with a helper phage. The term “Ff phage” is defined to be the set of filamentous bacteriophage including M13, fd, fl, and their numerous derivatives.

The term “Fc-region amino acid sequence” as used herein refers to an amino acid sequence comprising the Fc (fragment crystallizable) domain of an immunuglobulin molecule and includes fragments of this domain that exhibit an affinity to the novel binding molecules disclosed by the present invention. Equivalent terms used throughout this disclosure include “Fc-region target”, “Fc-region polypeptide” or simply “Fc”.

The term “binding molecule” as used herein refers to any molecule, polypeptide, peptidomimetic or transformed cell (“transformant”) capable of forming a binding complex with another molecule, polypeptide, peptidomimetic or tranformant. An “Fc-region binding molecule” is a binding molecule that forms a complex with an Fc-region polypeptide. Specific examples of Fc-region binding molecules are the polypeptides described herein (e.g., SEQ ID NOs: 4-7, 14-143, and 186-200) and bacteriophage displaying any of such polypeptides. Also included within the definition of Fc-region binding molecules are polypeptides derived from or including a polypeptide having an amino acid sequence according to formula I, II, III, or IV above, and such polypeptides which have been modified for particular results, e.g., for immobilization or labeling. Specific examples of modifications contemplated are C-terminal or N-terminal amino acid substitutions or polypeptide chain elongations for the purpose of linking the binding molecule to a chromatographic support or other substrate. Another example involves substitutions of pairs of cysteine residues that normally form disulfide links, for example with non-naturally occurring amino acid residues having reactive side chains, for the purpose of forming a more stable bond between those amino acid positions than the former disulfide bond. All such modified binding molecules are also considered binding molecules according to this invention so long as they retain the ability to bind Fc-region polypeptides.

As used herein, the term “isolated” when applied to a polypeptide according to the invention refers to a polypeptide that has been synthesized or that has been removed from its native environment, e.g., separated from other polypeptides or proteins with which it is naturally associated. In a preferred form, an isolated polypeptide is substantially free of other polypeptides, particularly other polypeptides of natural origin, for example it is provided in a highly purified form, i.e., greater than 95% pure, more preferably greater than 99% pure. When used in this context, the term “isolated” does not exclude the presence of the same polypeptide in alternative physical forms, such as dimers or alternatively glycosylated or derivatized forms.

As used herein, the term “solid support” includes any substrate suitable for the immobilization of polypeptides. Such substrates include cellulose, controlled-pore glass, silica, polystyrene, styrene divinyl benzene, agarose, and crosslinked agarose. Solid supports may take any of a variety of forms including but not limited to plates, filters, beads, resins, and the like. As used herein, the term “solid support” includes materials also known as “chromatographic matrix material”, which describes a wide variety of commercially available chromatographic materials known to be useful in separating or fractionating biological materials. Useful matrix materials include polymeric substances such as cellulose or silica gel-type resins or membranes or crosslinked polysaccharides such as agarose. In addition, the chromatographic matrix materials may further comprise various functional or active groups or molecules bound to the matrices that are useful in separating biological molecules such as, for instance, heparin, biotin, streptavidin, synthetic peptide ligands, or monoclonal antibodies. Particular examples of useful chromatographic matrix materials include but are not limited to aldehyde-functional methacrylate resins, in particular formyl methacrylate resins, and NHS-activated agarose. A wide variety of solid supports are available commercially from such suppliers as Amersham Pharmacia Biotech (Piscataway, N.J.).

As used herein, the term “affinity tag” indicates any molecule, such as biotin, with an affinity for a ligand, such as streptavidin, that can be conjugated with another molecule, to render that molecule capable of capture by binding to the ligand. Thus, a biotinylated polypeptide will have a biotin “affinity tag” making it suitable for isolation by contact with the biotin binding partner (ligand), streptavidin. Other examples of common affinity tags are polyhistidine (e.g., hexaHis), providing for affinity capture on a metal chelate (IMAC) resin, or a myc tag, which is an epitope of the c-myc protein, providing for affinity capture by immobilized anti-myc tag antibodies (such as monoclonal antibody 9E10, available from Berkely Antibody Co., Richmond, Calif.).

### DETAILED DESCRIPTION

The present invention provides novel binding molecules for immunoglobulin Fc-region amino acid sequences. Such molecules make possible the efficient detection, isolation, and purification of molecules that include Fc-region amino acid sequences. More particularly, such polypeptides may be useful for the detection, isolation, removal, and/or purification of antibodies from a solution containing them. Additionally, such polypeptides may be useful for the isolation of a protein or peptide of interest which is present in solution as one domain of a fusion protein, which fusion protein comprises an Fc-region polypeptide and preferably also a cleavage sequence, such as, for instance, an enterokinase cleavage sequence.

Also, the Fc-region binding polypeptides disclosed in the present application may be useful where it is desirable to regulate or prevent an antibody response by controlling the binding of the Fc-region of an antibody to the Fc receptors located on the surface of various cells involved in an immune response.

Use of Phage Display Technology

Phage display technology was advantageously used to identify novel Fc-region binding polypeptides according to the present invention. To prepare phage display libraries for such a use, a candidate binding domain is selected to serve as a structural template for the engineered binding molecules that will exhibit the desired binding and release capabilities. The binding domain may be a naturally occurring or synthetic protein, or a region or domain of a protein. The candidate binding domain may be selected based on knowledge of a known interaction between the candidate binding domain and the Fc-region amino acid sequence, but this is not critical. In fact, it is not essential that the candidate binding domain have any affinity for Fc-region amino acid sequences at all: its purpose is to provide a structure from which a multiplicity of analogues (a “library”) can be generated, which multiplicity of analogues will include one or more analogues that exhibit the desired binding and release properties (and any other properties selected for).

In selecting a candidate binding domain, the object is to provide a template or parental structure from which a library of similarly structured analogue domains can be generated. The analogue library will preferably be a biased library (as opposed to a randomly generated library), in that variegation of the basic domain to create the library will be carried out in such a way as to favor the properties desired for the binding molecules.

The nature of the candidate binding domain greatly influences the properties of the derived peptides (analogues) that will be tested against the Fc-region target. In selecting the candidate binding domain, the most important consideration is how the analogue domains will be presented to the Fc-region amino acid sequence, i.e., in what conformation the Fc-region amino acid sequence and the analogues will come into contact. In preferred embodiments, for example, the analogues will be generated by insertion of synthetic DNA encoding the analogue into a replicable genetic package, resulting in display of the domain on the surface of a microorganism, such as M13 phage. Such libraries of phage, such as M13 phage, displaying a wide variety of different polypeptides, can be prepared using techniques as described, e.g., in Kay et al., Phage Display of Peptides and Proteins: A Laboratory Manual (Academic Press, Inc., San Diego 1996) and U.S. Pat. No. 5,223,409 (Ladner et al.), both incorporated herein by reference in their entirety.

For formation of phage display libraries, it is preferred to use structured polypeptides as candidate binding domains, as opposed to unstructured peptides. Mutation of surface residues in a protein will usually have little effect on the overall structure or general properties (such as size, stability, and temperature of denaturation) of the protein; while at the same time mutation of surface residues may profoundly affect the binding properties of the protein. The more tightly a peptide segment is constrained (e.g., by disulfide linkages), the less likely it is to bind to any particular target. If it does bind, however, the binding is likely to be tighter and more specific. Thus, it is preferred to select a candidate binding domain and, in turn, a structure for the polypeptide analogues, that is constrained within a framework having some degree of rigidity.

The size of the candidate binding domain is also an important consideration. Small proteins or polypeptides offer several advantages over large proteins. First, the mass per binding site is reduced. Highly stable protein domains having low molecular weights, e.g., Kunitz domains (˜7 kDa), Kazal domains (˜7 kDa), Cucurbida maxima trypsin inhibitor (CMTI) domains (˜3.5 kDa), and endothelin (˜2 kDa), can show much higher binding per gram than do antibodies (150 kDa) or single-chain antibodies (30 kDa). Second, the possibility of non-specific binding is reduced because there is less surface available. Third, small proteins or polypeptides can be engineered to have unique tethering sites in a way that is impractical for larger proteins or antibodies. For example, small proteins can be engineered to have lysine residues only at sites suitable for tethering (e.g., to a chromatography matrix), but this is not feasible for antibodies. Fourth, a constrained polypeptide structure is more likely than a non-constrained structure to retain its functionality when transferred with the structural domain intact from one framework to another. For instance, the binding domain structure is likely to be transferable from the framework used for presentation in a library (e.g., displayed on a phage) to an isolated protein removed from the presentation framework or immobilized on a chromatographic substrate.

Immobilization of the polypeptides according to the invention is contemplated, e.g., onto chromatographic matrices to form efficient Fc-region polypeptide separation media for solutions such as whole blood, plasma, transgenic milk, eggs of transgenic chickens, etc. or conditioned culture media containing Fc-region peptides, e.g., any solution or feed stream that contains natural or synthetic immunoglobulins or Fc-containing fragments thereof. By selecting appropriate binding domain templates, binding polypeptides having a single free cysteine (i.e., a cysteine residue unpaired with another cysteine that ordinarily forms a disulfide link) can be isolated. Such thiol-functional polypeptides can be used for immobilization to substrates by formation of a thioether with iodoacetamide, iodoacetic acid, or similar α-iodo carboxylic acid groups. Similarly, the C-terminal carboxyl group of the peptide domain may be converted to a hydrazide (—NH—NH₂), for reaction with an aldehyde-functional substrate.

The candidate binding domain of the libraries described herein is a short cyclic peptide of 6-12 amino acids flanked by 2-3 variegated amino acids outside the invariant cysteine residues. Libraries based on these domains may be displayed on replicable genetic packages such as bacteria, yeast, and, preferably, bacteriophage, or “phage” (including “phagemid”) and can be readily constructed and used for the selection of binding molecules according to this invention.

Providing a Library of Candidate Binding Domain Analogues

Once a candidate binding domain has been selected, a library of potential binding molecules is created for screening against a target, in this case Fc-region polypeptides. The library is created by making a series of analogues, each analogue corresponding to the candidate binding domain except having one or more amino acid substitutions in the sequence of the domain.

The object of creating the analogue library is to provide a very large number of potential binding molecules for reaction with the Fc-region amino acid sequence molecule, and in general the greater the number of analogues in the library, the greater the likelihood that a member of the library will bind to the Fc-region amino acid sequence and release under the preselected conditions desired for release.

As indicated previously, the techniques discussed in Kay et al., Phage Display of Peptides and Proteins: A Laboratory Manual (Academic Press, Inc., San Diego 1996) or U.S. Pat. No. 5,223,409 are particularly useful in preparing a library of analogues corresponding to a selected candidate binding domain, which analogues will be presented in a form suitable for large-scale screening of large numbers of analogues with respect to a target Fc-region amino acid sequence. The use of replicable genetic packages, and most preferably bacteriophage, or “phage” (including “phagemid”) is a powerful method of generating novel polypeptide binding entities that involves introducing a novel, exogenous DNA segment into the genome of a bacteriophage (or other amplifiable genetic package) so that the polypeptide encoded by the non-native DNA appears on the surface of the phage. When the inserted DNA contains sequence diversity, then each recipient phage displays one variant of the template (or “parental”) amino acid sequence encoded by the DNA, and the phage population (library) displays a vast number of different but related amino acid sequences. The use of bacteriophage can also advantageously utilize phagemid vectors, as discussed, e.g., in Smith, Gene, 128:1 (1993), incorporated herein by reference in its entirety.

In a screening procedure to obtain Fc-region polypeptide binders according to this invention, a phage library is contacted with and allowed to bind a target Fc-region polypeptide, usually immobilized on a solid support. Non-binders are separated from binders. In various ways, the bound phage are liberated from the Fc-region peptide, collected and amplified. Since the phage can be amplified through infection of bacterial cells, even a few binding phage are sufficient to reveal the gene sequence that encodes a binding entity. After a first set of binding peptides is identified, the sequence information can be used to design other libraries biased for members having additional desired properties, e.g., discrimination between Fc-region polypeptides and particular fragments or closely related impurities in a particular feed stream.

Such techniques make it possible not only to screen a large number of potential Fc-region binding molecules but make it practical to repeat the binding/elution cycles and to build secondary, biased libraries for screening analogue-displaying packages that meet initial criteria. Using these techniques, analogue biased libraries may be screened to reveal members that (a) bind tightly (i.e., with high affinity) under the binding conditions and (b) release cleanly (i.e., readily dissociate from the Fc-region peptide target) under preselected release conditions.

In isolating the specific polypeptides according to this invention, six phage display libraries were screened. The six libraries each displayed a short, variegated exogenous peptide loop of 6, 7, 8, 9, 10, or 12 amino acids on the surface of M13 phage, at the amino terminus of protein III. The libraries are designated TN6/6 (having a potential 3.3×10¹² amino acid sequence diversity); TN7/4 (having a potential 1.2×10¹⁴ amino acid sequence diversity), TN8/9 (having a potential 2.2×10¹⁵ amino acid sequence diversity), TN9/4 (having a potential 4.2×10¹⁶ amino acid sequence diversity, TN10/9 (having a potential 3.0×10¹⁶ amino acid sequence diversity), and TN12/1 (having a sequence diversity of 4.6×10¹⁹).

The TN6/6 library was constructed to display a single microprotein binding loop contained in a 12-amino acid template. The TN6/6 library utilized a template sequence of Xaa-Xaa-Xaa-Cys-Xaa-Xaa-Xaa-Xaa-Cys-Xaa-Xaa-Xaa (SEQ ID NO:8). The amino acids at positions 2, 3, 5, 6, 7, 8, 10, and 11 of the template were varied to permit any amino acid except cysteine (Cys). The amino acids at positions 1 and 12 of the template were varied to permit any amino acid except cysteine (Cys), glutamic acid (Glu), isoleucine (Ile), Lysine (Lys), methionine (Met), and threonine (Thr).

The TN7/4 library was constructed to display a single microprotein binding loop contained in a 13-amino acid template. The TN7/4 library utilized a template sequence of Xaa-Xaa-Xaa-Cys-Xaa-Xaa-Xaa-Xaa-Xaa-Cys-Xaa-Xaa-Xaa (SEQ ID NO:9). The amino acids at amino acid positions 1, 2, 3, 5, 6, 7, 8, 9, 11, 12, and 13 of the template were varied to permit any amino acid except cysteine (Cys).

The TN8/9 library was constructed to display a single microprotein binding loop contained in a 14-amino acid template. The TN8/9 library utilized a template sequence of Xaa-Xaa-Xaa-Cys-Xaa-Xaa-Xaa-Xaa-Xaa-Xaa-Cys-Xaa-Xaa-Xaa (SEQ ID NO:10). The amino acids at position 1, 2, 3, 5, 6, 7, 8, 9, 10, 12, 13, and 14 in the template were varied to permit any amino acid except cysteine (Cys).

The TN9/4 library was constructed to display a single microprotein binding loop contained in an 15-amino acid template. The TN9/1 library utilized a template sequence Xaa-Xaa-Xaa-Cys-Xaa-Xaa-Xaa-Xaa-Xaa-Xaa-Xaa-Cys-Xaa-Xaa-Xaa (SEQ ID NO:11). The amino acids at position 1, 2, 3, 5, 6, 7, 8, 9, 10, 11, 13, 14 and 15 in the template were varied to permit any amino acid except cysteine (Cys).

The TN10/9 library was constructed to display a single microprotein binding loop contained in a 16-amino acid template. The TN10/9 library utilized a template sequence Xaa-Xaa-Xaa-Cys-Xaa-Xaa-Xaa-Xaa-Xaa-Xaa-Xaa-Xaa-Cys-Xaa-Xaa-Xaa (SEQ ID NO:12). The amino acids at positions 1, 2, 15, and 16 in the template were varied to permit any amino acid selected from a group of 10 amino acids: D, F, H, L, N, P, R, S, W, or Y). The amino acids at positions 3 and 14 in the template were varied to permit any amino acid selected from a group of 14 amino acids: A, D, F, G, H, L, N, P, Q, R, S, V, W, or Y). The amino acids at positions 5, 6, 7, 8, 9, 10, 11, and 12 in the template were varied to permit any amino acid except cysteine (Cys).

The TN12/1 library was constructed to display a single microprotein binding loop contained in an 18-amino acid template. The TN12/1 library utilized a template sequence Xaa-Xaa-Xaa-Cys-Xaa-Xaa-Xaa-Xaa-Xaa-Xaa-Xaa-Xaa-Xaa-Xaa-Cys-Xaa-Xaa-Xaa (SEQ ID NO:13). The amino acids at position 1, 2, 17, and 18 in the template were varied to permit any amino acid selected from a group of 12 amino acids: A, D, F, G, H, L, N, P, R, S, W, or Y). The amino acids at positions 3, 5, 6, 7, 8, 9, 10, 11, 12, 13, 14, and 16 were varied to permit any amino acid except cysteine (Cys).

The foregoing phage display libraries displaying cyclic peptides of from 6 to 12 amino acid residues were screened to reveal members binding to Fc-region peptide targets. A number of Fc-region amino acid sequence-binding polypeptides were isolated, with Fc-region binders being isolated from five of the libraries screened:

| | |
|---|---|
| TN12/1 | |
| R-R-A-C-S-R-D-W-S-G-A-L-V-W-C-A-G- | (SEQ ID NO:14) |

H

| | |
|---|---|
| W-G-E-C-T-V-T-S-Y-G-E-L-I-W-C-G-G- | (SEQ ID NO:15) |

L

| | |
|---|---|
| S-S-A-C-A-F-D-P-M-G-A-V-I-W-C-T-Y- | (SEQ ID NO:16) |

D

| | |
|---|---|
| L-L-E-C-A-Y-N-T-S-G-E-L-I-W-C-N-G- | (SEQ ID NO:17) |

S

| | |
|---|---|
| P-D-D-C-S-I-H-F-S-G-E-L-I-W-C-E-P- | (SEQ ID NO:18) |

L

| | |
|---|---|
| L-G-E-C-T-V-T-S-Y-G-E-L-I-W-C-G-G- | (SEQ ID NO:19) |

L

| | |
|---|---|
| W-G-E-C-T-V-T-S-Y-G-E-L-I-W-C-G-G- | (SEQ ID NO:20) |

H

| | |
|---|---|
| D-H-M-C-V-Y-T-T-W-G-E-L-I-W-C-D-D- | (SEQ ID NO:21) |

H

| | |
|---|---|
| W-G-E-C-T-V-T-S-Y-G-E-L-I-W-C-G-G- | (SEQ ID NO:22) |

L

| | |
|---|---|
| C-R-A-C-S-R-D-W-P-G-A-L-V-W-C-A-G- | (SEQ ID NO:23) |

H

| | |
|---|---|
| R-R-A-C-S-R-D-W-S-G-A-L-V-W-C-A-G- | (SEQ ID NO:24) |

H

| | |
|---|---|
| L-H-A-C-A-F-D-P-M-G-A-V-I-W-C-T-Y- | (SEQ ID NO:25) |

D

| | |
|---|---|
| D-H-M-C-V-Y-T-T-W-G-E-L-M-W-C-D-N- | (SEQ ID NO:26) |

H

| | |
|---|---|
| P-P-T-C-T-W-D-W-Q-G-I-L-V-W-C-S-G- | (SEQ ID NO:27) |

H

| | |
|---|---|
| S-N-K-C-S-N-T-W-D-G-S-L-I-W-C-S-A- | (SEQ ID NO:28) |

N

| | |
|---|---|
| F-P-E-C-T-F-D-M-E-G-F-L-I-W-C-S-S- | (SEQ ID NO:29) |

F

| | |
|---|---|
| H-D-L-C-A-Q-A-P-F-G-D-A-T-W-C-D-L- | (SEQ ID NO:30) |

R

| | |
|---|---|
| P-N-H-C-S-Y-N-L-K-S-E-L-I-W-C-Q-D- | (SEQ ID NO:31) |

L

| | |
|---|---|
| P-L-D-C-A-R-D-I-H-N-S-L-I-W-C-S-L- | (SEQ ID NO:32) |

G

| | |
|---|---|
| G-S-E-C-S-W-T-S-L-N-E-L-I-W-C-A-H- | (SEQ ID NO:33) |

W

| | |
|---|---|
| W-P-D-C-S-F-T-V-Q-R-D-L-I-W-C-E-A- | (SEQ ID NO:34) |

L

| | |
|---|---|
| S-H-S-C-A-Y-D-Y-A-H-M-L-V-W-C-T-H- | (SEQ ID NO:35) |

F

| | |
|---|---|
| D-H-M-C-V-Y-T-T-W-G-E-L-I-W-C-D-N- | (SEQ ID NO:36) |

H

| | |
|---|---|
| R-P-N-C-T-F-A-A-S-G-E-L-I-W-C-M-H- | (SEQ ID NO:37) |

Y

| | |
|---|---|
| W-W-G-C-Q-F-D-W-R-G-E-L-V-W-C-P-Y- | (SEQ ID NO:38) |

L

| | |
|---|---|
| G-G-V-C-S-Y-S-G-M-G-E-I-V-W-C-R-W- | (SEQ ID NO:39) |

F

| | |
|---|---|
| A-L-M-C-S-H-D-M-W-G-S-L-I-W-C-K-H- | (SEQ ID NO:40) |

F

| | |
|---|---|
| W-W-N-C-H-N-G-W-T-W-T-G-G-W-C-W-W- | (SEQ ID NO:41) |

F

| | |
|---|---|
| Y-H-V-C-A-R-D-S-W-D-Q-L-I-W-C-E-A- | (SEQ ID NO:42) |

F

| | |
|---|---|
| L-Y-W-C-H-V-W-F-G-Q-H-A-W-Q-C-K-Y- | (SEQ ID NO:48) |

P

| | |
|---|---|
| D-D-H-C-Y-W-F-R-E-W-F-N-S-E-C-P-H- | (SEQ ID NO:50) |

G

| | |
|---|---|
| S-G-D-C-G-F-W-P-R-I-W-G-L-C-M-D-N | (SEQ ID NO:54) |
| A-A-T-C-S-T-S-Y-W-Y-Y-Q-W-F-C-T-D- | (SEQ ID NO:124) |

S

| | |
|---|---|
| D-W-Q-C-L-W-W-G-N-S-F-W-P-Y-C-A-N- | (SEQ ID NO:127) |

L

| | |
|---|---|
| N-P-M-C-W-K-K-S-W-W-E-D-A-Y-C-I-N- | (SEQ ID NO:129) |

H

| | |
|---|---|
| S-W-V-C-W-K-A-K-W-W-E-D-K-R-C-A-P- | (SEQ ID NO:130) |

F

| | |
|---|---|
| S-R-Q-C-W-K-E-L-W-W-T-D-Q-M-C-L-D- | (SEQ ID NO:131) |

L

| | |
|---|---|
| D-G-V-C-G-P-R-G-F-G-P-A-W-F-C-M-I- | (SEQ ID NO:135) |

T-Y

| | |
|---|---|
| P-G-W-C-Y-S-D-I-W-G-F-K-H-F-C-N-L- | (SEQ ID NO:138) |

D

| | |
|---|---|
| S-W-N-C-A-F-H-H-N-E-M-V-W-C-D-D-G | (SEQ ID NO:142) |
| G-S-K-C-K-Q-T-G-F-P-R-W-W-C-E-H-Y | (SEQ ID NO:134) |
| TN10/9 | |
| Y-W-Y-C-K-W-F-S-E-S-A-S-C-S-S-R | (SEQ ID NO:44) |
| Y-W-Y-C-K-W-F-E-D-K-H-P-C-D-S-S | (SEQ ID NO:45) |
| Y-W-Y-C-S-W-F-P-D-R-P-D-C-P-L-Y | (SEQ ID NO:46) |
| N-Y-W-C-N-V-W-L-L-G-D-V-C-R-S-H | (SEQ ID NO:47) |
| Y-W-F-C-Q-W-F-S-Q-N-H-T-C-F-R-D | (SEQ ID NO:52) |
| H-Y-W-C-D-I-W-F-G-A-P-A-C-Q-F-R | (SEQ ID NO:53) |
| F-W-Y-C-K-W-F-Y-E-D-A-Q-C-S-H-D | (SEQ ID NO:55) |
| Y-W-Y-C-Q-W-F-Q-E-V-N-K-C-F-N-S | (SEQ ID NO:120) |
| Y-Y-W-C-R-H-W-F-P-D-F-D-C-V-H-S | (SEQ ID NO:121) |
| Y-W-Y-C-S-W-F-P-D-R-P-D-C-P-L-Y | (SEQ ID NO:122) |
| Y-W-Y-C-V-W-F-D-N-A-D-Q-C-V-H-H | (SEQ ID NO:123) |
| S-F-R-C-Q-S-S-F-P-S-W-Y-C-D-Y-Y | (SEQ ID NO:132) |
| S-W-H-C-Q-N-T-Y-P-E-W-Y-C-Q-W-Y | (SEQ ID NO:133) |
| Y-S-H-C-A-T-H-Y-P-T-W-Y-C-L-H-F | (SEQ ID NO:136) |
| F-C-N-C-W-G-S-H-E-F-T-F-C-V-D-D | (SEQ ID NO:137) |
| D-S-S-C-I-K-H-H-N-K-V-T-C-F-F-P | (SEQ ID NO:139) |
| TN9/4 | |
| N-Y-W-C-N-F-W-Q-L-P-T-C-D-N-L | (SEQ ID NO:43) |
| N-Y-W-C-N-I-W-G-L-H-G-C-N-S-H | (SEQ ID NO:51) |
| N-Y-W-C-N-F-W-Q-L-P-T-C-D-N-L | (SEQ ID NO:119) |
| Y-W-Y-C-W-F-P-D-R-P-E-C-P-L-Y | (SEQ ID NO:143) |
| TN8/9 | |
| Y-W-K-C-K-W-M-P-W-M-C-G-F-D | (SEQ ID NO:49) |
| E-Y-W-C-K-Y-W-G-L-E-C-V-H-R | (SEQ ID NO:86) |
| K-Y-W-C-T-Q-W-G-L-K-C-D-K-Q | (SEQ ID NO:87) |
| R-Y-W-C-N-F-W-G-V-N-C-D-A-N | (SEQ ID NO:89) |
| N-Y-W-C-T-H-W-G-V-M-C-L-D-H | (SEQ ID NO:90) |
| Y-W-F-C-K-W-F-P-S-Q-C-Q-F-M | (SEQ ID NO:91) |
| A-Y-W-C-K-Q-W-G-L-K-C-Q-L-G | (SEQ ID NO:92) |
| K-Y-W-C-K-F-W-G-L-E-C-K-V-G | (SEQ ID NO:93) |
| N-Y-W-C-T-E-W-G-L-N-C-N-N-K | (SEQ ID NO:94) |
| S-Y-W-C-E-K-W-G-L-T-C-E-T-H | (SEQ ID NO:95) |
| E-Y-W-C-R-I-W-G-L-Q-C-N-M-V | (SEQ ID NO:96) |
| K-Y-W-C-K-K-W-G-V-N-C-D-F-N | (SEQ ID NO:97) |
| K-Y-W-C-S-V-W-G-V-Q-C-P-H-S | (SEQ ID NO:98) |
| F-Y-W-C-T-K-W-G-L-E-C-I-H-S | (SEQ ID NO:99) |
| H-Y-W-C-Q-Q-W-G-L-M-C-F-E-T | (SEQ ID NO:100) |
| K-Y-W-C-K-R-W-G-L-M-C-N-G-G | (SEQ ID NO:101) |
| A-Y-W-C-M-T-W-G-V-P-C-I-S-W | (SEQ ID NO:102) |
| K-Y-W-C-K-K-W-G-V-N-C-D-F-N | (SEQ ID NO:103) |
| K-Y-W-C-S-V-W-G-V-Q-C-P-D-S | (SEQ ID NO:104) |
| K-Y-W-C-S-V-W-G-V-Q-C-P-H-S | (SEQ ID NO:105) |
| L-Y-W-C-T-K-W-G-V-T-C-Q-K-D | (SEQ ID NO:106) |
| T-Y-W-C-H-K-W-G-V-K-C-A-T-T | (SEQ ID NO:107) |
| T-Y-W-C-T-F-W-E-L-P-C-D-P-A | (SEQ ID NO:108) |
| K-Y-W-C-T-K-W-Q-L-N-C-E-E-V | (SEQ ID NO:109) |
| N-Y-W-C-H-F-W-Q-V-P-C-L-E-Q | (SEQ ID NO:110) |
| T-Y-W-C-V-V-W-N-V-P-C-S-T-D | (SEQ ID NO:111) |
| N-F-W-C-H-T-W-G-L-Q-C-N-D-L | (SEQ ID NO:112) |
| F-W-Y-C-Y-W-F-N-E-K-C-K-T-P | (SEQ ID NO:113) |
| G-F-W-C-T-F-W-G-V-T-C-E-A-G | (SEQ ID NO:114) |
| P-H-N-C-D-D-H-Y-W-Y-C-K-W-F | (SEQ ID NO:115) |
| E-M-T-C-S-S-H-Y-W-Y-C-T-W-M | (SEQ ID NO:116) |
| H-I-D-C-K-T-N-Y-W-W-C-R-W-T | (SEQ ID NO:117) |
| E-M-R-C-G-Q-H-F-W-Y-C-E-W-F | (SEQ ID NO:118) |
| Y-W-A-C-V-W-G-L-K-S-C-V-D-R | (SEQ ID NO:125) |
| F-W-R-C-H-W-W-P-E-R-C-P-V-D | (SEQ ID NO:128) |
| P-V-D-C-K-H-H-F-W-W-C-Y-W-N | (SEQ ID NO:141) |
| TN7/4 | |
| Y-Y-W-C-N-Y-W-G-L-C-P-D-Q | (SEQ ID NO:56) |
| S-Y-W-C-K-I-W-D-V-C-P-Q-S | (SEQ ID NO:57) |
| K-Y-W-C-N-L-W-G-V-C-P-A-N | (SEQ ID NO:58) |
| Q-Y-W-C-Y-Q-W-G-L-C-G-A-N | (SEQ ID NO:59) |
| K-Y-W-C-Q-Q-W-G-V-C-N-G-S | (SEQ ID NO:60) |
| K-Y-W-C-V-Q-W-G-V-C-P-E-S | (SEQ ID NO:61) |
| K-Y-W-C-M-Q-W-G-L-C-G-W-E | (SEQ ID NO:62) |
| H-F-W-C-E-V-W-G-L-C-P-S-I | (SEQ ID NO:63) |
| Q-Y-W-C-T-K-W-G-L-C-T-N-V | (SEQ ID NO:64) |
| A-Y-W-C-K-V-W-G-L-C-Q-G-E | (SEQ ID NO:65) |
| K-Y-W-C-N-L-W-G-V-C-P-A-N | (SEQ ID NO:66) |
| Q-Y-W-C-N-V-W-G-V-C-L-P-S | (SEQ ID NO:67) |
| H-Y-W-C-Q-Q-W-G-I-C-E-R-P | (SEQ ID NO:68) |
| R-Y-W-C-N-I-W-D-V-C-P-E-Q | (SEQ ID NO:69) |
| Q-Y-W-C-T-H-W-G-L-C-G-K-Y | (SEQ ID NO:70) |
| T-Y-W-C-T-K-W-G-L-C-P-H-N | (SEQ ID NO:71) |
| F-Y-W-C-G-Q-W-G-L-C-A-P-P | (SEQ ID NO:72) |
| G-Y-W-C-N-V-W-G-L-C-S-T-E | (SEQ ID NO:73) |
| R-Y-W-C-G-V-W-G-V-C-E-I-D | (SEQ ID NO:74) |
| K-F-W-C-T-I-W-G-V-C-H-M-P | (SEQ ID NO:75) |
| H-Y-W-C-Q-Q-W-G-I-C-E-R-P | (SEQ ID NO:76) |
| R-Y-W-C-N-I-W-D-V-C-P-E-Q | (SEQ ID NO:77) |
| F-Y-W-C-S-Q-W-G-L-C-K-Y-D | (SEQ ID NO:78) |
| H-Y-W-C-E-K-W-G-L-C-L-M-S | (SEQ ID NO:79) |
| H-Y-W-C-Q-K-W-G-V-C-P-T-D | (SEQ ID NO:80) |
| H-Y-W-C-S-L-W-G-V-C-D-I-N | (SEQ ID NO:81) |
| R-F-W-C-S-A-W-G-V-C-P-A | (SEQ ID NO:82) |
| S-Y-W-C-K-I-W-D-V-C-P-Q-S | (SEQ ID NO:83) |
| Q-Y-W-C-S-I-W-K-V-C-P-G-R | (SEQ ID NO:84) |
| Y-W-Y-C-E-W-F-G-A-C-I-N-D | (SEQ ID NO:85) |
| K-Y-W-C-S-F-W-G-L-Q-C-K-T | (SEQ ID NO:88) |
| Y-W-R-C-V-W-F-P-A-S-C-P-T | (SEQ ID NO:126) |
| R-W-S-C-W-G-V-W-G-C-V-W-V | (SEQ ID NO:140) |

Further analysis of recurring amino acid sequences among the Fc binders revealed a series of polypeptide “families” exhibiting common core structures:

| | |
|---|---|
| Sequence Family I: | |
| R-R-A-C-S-R-D-W-S-G-A-L-V-W-C-A-G-H | (SEQ ID NO:14) |
| W-G-E-C-T-V-T-S-Y-G-E-L-I-W-C-G-G-L | (SEQ ID NO:15) |
| S-S-A-C-A-F-D-P-M-G-A-V-I-W-C-T-Y-D | (SEQ ID NO:16) |
| L-L-E-C-A-Y-N-T-S-G-E-L-I-W-C-N-G-S | (SEQ ID NO:17) |
| P-D-D-C-S-I-H-F-S-G-E-L-I-W-C-E-P-L | (SEQ ID NO:18) |
| L-G-E-C-T-V-T-S-Y-G-E-L-I-W-C-G-G-L | (SEQ ID NO:19) |
| W-G-E-C-T-V-T-S-Y-G-E-L-I-W-C-G-G-H | (SEQ ID NO:20) |
| D-H-M-C-V-Y-T-T-W-G-E-L-I-W-C-D-D-H | (SEQ ID NO:21) |
| W-G-E-C-T-V-T-S-Y-G-E-L-I-W-C-G-G-L | (SEQ ID NO:22) |
| C-R-A-C-S-R-D-W-P-G-A-L-V-W-C-A-G-H | (SEQ ID NO:23) |
| R-R-A-C-S-R-D-W-S-G-A-L-V-W-C-A-G-H | (SEQ ID NO:24) |
| L-H-A-C-A-F-D-P-M-G-A-V-I-W-C-T-Y-D | (SEQ ID NO:25) |
| D-H-M-C-V-Y-T-T-W-G-E-L-M-W-C-D-N-H | (SEQ ID NO:26) |
| P-P-T-C-T-W-D-W-Q-G-I-L-V-W-C-S-G-H | (SEQ ID NO:27) |
| S-N-K-C-S-N-T-W-D-G-S-L-I-W-C-S-A-N | (SEQ ID NO:28) |
| F-P-E-C-T-F-D-M-E-G-F-L-I-W-C-S-S-F | (SEQ ID NO:29) |
| H-D-L-C-A-Q-A-P-F-G-D-A-T-W-C-D-L-R | (SEQ ID NO:30) |
| P-N-H-C-S-Y-N-L-K-S-E-L-I-W-C-Q-D-L | (SEQ ID NO:31) |
| P-L-D-C-A-R-D-I-H-N-S-L-I-W-C-S-L-G | (SEQ ID NO:32) |
| G-S-E-C-S-W-T-S-L-N-E-L-I-W-C-A-H-W | (SEQ ID NO:33) |
| W-P-D-C-S-F-T-V-Q-R-D-L-I-W-C-E-A-L | (SEQ ID NO:34) |
| S-H-S-C-A-Y-D-Y-A-H-M-L-V-W-C-T-H-F | (SEQ ID NO:35) |
| D-H-M-C-V-Y-T-T-W-G-E-L-I-W-C-D-N-H | (SEQ ID NO:36) |
| R-P-N-C-T-F-A-A-S-G-E-L-I-W-C-M-H-Y | (SEQ ID NO:37) |
| W-W-G-C-Q-F-D-W-R-G-E-L-V-W-C-P-Y-L | (SEQ ID NO:38) |
| G-G-V-C-S-Y-S-G-M-G-E-I-V-W-C-R-W-F | (SEQ ID NO:39) |
| A-L-M-C-S-H-D-M-W-G-S-L-I-W-C-K-H-F | (SEQ ID NO:40) |
| W-W-N-C-H-N-G-W-T-W-T-G-G-W-C-W-W-F | (SEQ ID NO:41) |
| Y-H-V-C-A-R-D-S-W-D-Q-L-I-W-C-E-A-F | (SEQ ID NO:42) |
| Sequence Family II: | |
| N-Y-W-C-N-F-W-Q-L-P-T-C-D-N-L | (SEQ ID NO:43) |
| Y-W-Y-C-K-W-F-S-E-S-A-S-C-S-S-R | (SEQ ID NO:44) |
| Y-W-Y-C-K-W-F-E-D-K-H-P-C-D-S-S | (SEQ ID NO:45) |
| Y-W-Y-C-S-W-F-P-D-R-P-D-C-P-L-Y | (SEQ ID NO:46) |
| N-Y-W-C-N-V-W-L-L-G-D-V-C-R-S-H | (SEQ ID NO:47) |
| L-Y-W-C-H-V-W-F-G-Q-H-A-W-Q-C-K-Y-P | (SEQ ID NO:48) |
| Y-W-K-C-K-W-M-P-W-M-C-G-F-D | (SEQ ID NO:49) |
| D-D-H-C-Y-W-F-R-E-W-F-N-S-E-C-P-H-G | (SEQ ID NO:50) |
| N-Y-W-C-N-I-W-G-L-H-G-C-N-S-H | (SEQ ID NO:51) |
| Y-W-F-C-Q-W-F-S-Q-N-H-T-C-F-R-D | (SEQ ID NO:52) |
| H-Y-W-C-D-I-W-F-G-A-P-A-C-Q-F-R | (SEQ ID NO:53) |
| S-G-D-C-G-F-W-P-R-I-W-G-L-C-M-D-N | (SEQ ID NO:54) |
| F-W-Y-C-K-W-F-Y-E-D-A-Q-C-S-H-D | (SEQ ID NO:55) |
| Y-Y-W-C-N-Y-W-G-L-C-P-D-Q | (SEQ ID NO:56) |
| S-Y-W-C-K-I-W-D-V-C-P-Q-S | (SEQ ID NO:57) |
| K-Y-W-C-N-L-W-G-V-C-P-A-N | (SEQ ID NO:58) |
| Q-Y-W-C-Y-Q-W-G-L-C-G-A-N | (SEQ ID NO:59) |
| K-Y-W-C-Q-Q-W-G-V-C-N-G-S | (SEQ ID NO:60) |
| K-Y-W-C-V-Q-W-G-V-C-P-E-S | (SEQ ID NO:61) |
| K-Y-W-C-M-Q-W-G-L-C-G-W-E | (SEQ ID NO:62) |
| H-F-W-C-E-V-W-G-L-C-P-S-I | (SEQ ID NO:63) |
| Q-Y-W-C-T-K-W-G-L-C-T-N-V | (SEQ ID NO:64) |
| A-Y-W-C-K-V-W-G-L-C-Q-G-E | (SEQ ID NO:65) |
| K-Y-W-C-N-L-W-G-V-C-P-A-N | (SEQ ID NO:66) |
| Q-Y-W-C-N-V-W-G-V-C-L-P-S | (SEQ ID NO:67) |
| H-Y-W-C-Q-Q-W-G-I-C-E-R-P | (SEQ ID NO:68) |
| R-Y-W-C-N-I-W-D-V-C-P-E-Q | (SEQ ID NO:69) |
| Q-Y-W-C-T-H-W-G-L-C-G-K-Y | (SEQ ID NO:70) |
| T-Y-W-C-T-K-W-G-L-C-P-H-N | (SEQ ID NO:71) |
| F-Y-W-C-G-Q-W-G-L-C-A-P-P | (SEQ ID NO:72) |
| G-Y-W-C-N-V-W-G-L-C-S-T-E | (SEQ ID NO:73) |
| R-Y-W-C-G-V-W-G-V-C-E-I-D | (SEQ ID NO:74) |
| K-F-W-C-T-I-W-G-V-C-H-M-P | (SEQ ID NO:75) |
| H-Y-W-C-Q-Q-W-G-I-C-E-R-P | (SEQ ID NO:76) |
| R-Y-W-C-N-I-W-D-V-C-P-E-Q | (SEQ ID NO:77) |
| F-Y-W-C-S-Q-W-G-L-C-K-Y-D | (SEQ ID NO:78) |
| H-Y-W-C-E-K-W-G-L-C-L-M-S | (SEQ ID NO:79) |
| H-Y-W-C-Q-K-W-G-V-C-P-T-D | (SEQ ID NO:80) |
| H-Y-W-C-S-L-W-G-V-C-D-I-N | (SEQ ID NO:81) |
| R-F-W-C-S-A-W-G-V-C-P-A | (SEQ ID NO:82) |
| S-Y-W-C-K-I-W-D-V-C-P-Q-S | (SEQ ID NO:83) |
| Q-Y-W-C-S-I-W-K-V-C-P-G-R | (SEQ ID NO:84) |
| Y-W-Y-C-E-W-F-G-A-C-I-N-D | (SEQ ID NO:85) |
| E-Y-W-C-K-Y-W-G-L-E-C-V-H-R | (SEQ ID NO:86) |
| K-Y-W-C-T-Q-W-G-L-K-C-D-K-Q | (SEQ ID NO:87) |
| K-Y-W-C-S-F-W-G-L-Q-C-K-T | (SEQ ID NO:88) |
| R-Y-W-C-N-F-W-G-V-N-C-D-A-N | (SEQ ID NO:89) |
| N-Y-W-C-T-H-W-G-V-M-C-L-D-H | (SEQ ID NO:90) |
| Y-W-F-C-K-W-F-P-S-Q-C-Q-F-M | (SEQ ID NO:91) |
| A-Y-W-C-K-Q-W-G-L-K-C-Q-L-G | (SEQ ID NO:92) |
| K-Y-W-C-K-F-W-G-L-E-C-K-V-G | (SEQ ID NO:93) |
| N-Y-W-C-T-E-W-G-L-N-C-N-N-K | (SEQ ID NO:94) |
| S-Y-W-C-E-K-W-G-L-T-C-E-T-H | (SEQ ID NO:95) |
| E-Y-W-C-R-I-W-G-L-Q-C-N-M-V | (SEQ ID NO:96) |
| K-Y-W-C-K-K-W-G-V-N-C-D-F-N | (SEQ ID NO:97) |
| K-Y-W-C-S-V-W-G-V-Q-C-P-H-S | (SEQ ID NO:98) |
| F-Y-W-C-T-K-W-G-L-E-C-I-H-S | (SEQ ID NO:99) |
| H-Y-W-C-Q-Q-W-G-L-M-C-F-E-T | (SEQ ID NO:100) |
| K-Y-W-C-K-R-W-G-L-M-C-N-G-G | (SEQ ID NO:101) |
| A-Y-W-C-M-T-W-G-V-P-C-I-S-W | (SEQ ID NO:102) |
| K-Y-W-C-K-K-W-O-V-N-C-D-F-N | (SEQ ID NO:103) |
| K-Y-W-C-S-V-W-G-V-Q-C-P-D-S | (SEQ ID NO:104) |
| K-Y-W-C-S-V-W-G-V-Q-C-P-H-S | (SEQ ID NO:105) |
| L-Y-W-C-T-K-W-G-V-T-C-Q-K-D | (SEQ ID NO:106) |
| T-Y-W-C-H-K-W-G-V-K-C-A-T-T | (SEQ ID NO:107) |
| T-Y-W-C-T-F-W-E-L-P-C-D-P-A | (SEQ ID NO:108) |
| K-Y-W-C-T-K-W-Q-L-N-C-E-E-V | (SEQ ID NO:109) |
| N-Y-W-C-H-F-W-Q-V-P-C-L-E-Q | (SEQ ID NO:110) |
| T-Y-W-C-V-V-W-N-V-P-C-S-T-D | (SEQ ID NO:111) |
| N-F-W-C-H-T-W-G-L-Q-C-N-D-L | (SEQ ID NO:112) |
| F-W-Y-C-Y-W-F-N-E-K-C-K-T-P | (SEQ ID NO:113) |
| G-F-W-C-T-F-W-G-V-T-C-E-A-G | (SEQ ID NO:114) |
| P-H-N-C-D-D-H-Y-W-Y-C-K-W-F | (SEQ ID NO:115) |
| E-M-T-C-S-S-H-Y-W-Y-C-T-W-M | (SEQ ID NO:116) |
| H-I-D-C-K-T-N-Y-W-W-C-R-W-T | (SEQ ID NO:117) |
| E-M-R-C-G-Q-H-F-W-Y-C-E-W-F | (SEQ ID NO:118) |
| N-Y-W-C-N-F-W-Q-L-P-T-C-D-N-L | (SEQ ID NO:119) |
| Y-W-Y-C-Q-W-F-Q-E-V-N-K-C-F-N-S | (SEQ ID NO:120) |
| Y-Y-W-C-R-H-W-F-P-D-F-D-C-V-H-S | (SEQ ID NO:121) |
| Y-W-Y-C-S-W-F-P-D-R-P-D-C-P-L-Y | (SEQ ID NO:122) |
| Y-W-Y-C-V-W-F-D-N-A-D-Q-C-V-H-H | (SEQ ID NO:123) |
| A-A-T-C-S-T-S-Y-W-Y-Y-Q-W-F-C-T-D-S | (SEQ ID NO:124) |
| Y-W-A-C-V-W-G-L-K-S-C-V-D-R | (SEQ ID NO:125) |
| Y-W-R-C-V-W-F-P-A-S-C-P-T | (SEQ ID NO:126) |
| Sequence Family III: | |
| D-W-Q-C-L-W-W-G-N-S-F-W-P-Y-C-A-N-L | (SEQ ID NO:127) |
| F-W-R-C-H-W-W-P-E-R-C-P-V-D | (SEQ ID NO:128) |
| N-P-M-C-W-K-K-S-W-W-E-D-A-Y-C-I-N-H | (SEQ ID NO:129) |
| S-W-V-C-W-K-A-K-W-W-E-D-K-R-C-A-P-F | (SEQ ID NO:130) |
| S-R-Q-C-W-K-E-L-W-W-T-D-Q-M-C-L-D-L | (SEQ ID NO:131) |
| Sequence Family IV: | |
| S-F-R-C-Q-S-S-F-P-S-W-Y-C-D-Y-Y | (SEQ ID NO:132) |
| S-W-H-C-Q-N-T-Y-P-E-W-Y-C-Q-W-Y | (SEQ ID NO:133) |
| G-S-K-C-K-Q-T-G-F-P-R-W-W-C-E-H-Y | (SEQ ID NO:134) |
| D-G-V-C-G-P-R-G-F-G-P-A-W-F-C-M-H-Y | (SEQ ID NO:135) |
| Y-S-H-C-A-T-H-Y-P-T-W-Y-C-L-H-F | (SEQ ID NO:136) |

In the foregoing peptide families, the amino acids in bold type are either invariant at that position or are preferred (i.e., recurrent in multiple sequences) in a position relative to an invariant residue.

Fc-region binding peptides not fitting any family

(SEQ ID NO:137)

F-C-N-C-W-G-S-H-E-F-T-F-C-V-D-D

(SEQ ID NO:138)

P-G-W-C-Y-S-D-I-W-G-F-K-H-F-C-N-L-D

(SEQ ID NO:139)

D-S-S-C-I-K-H-H-N-K-V-T-C-F-F-P

(SEQ ID NO:140)

R-W-S-C-W-G-V-W-G-C-V-W-V

(SEQ ID NO:141)

P-V-D-C-K-H-H-F-W-W-C-Y-W-N

(SEQ ID NO:142)

S-W-N-C-A-F-H-H-N-E-M-V-W-C-D-D-G

(SEQ ID NO:143)

Y-W-Y-C-W-F-P-D-R-P-E-C-P-L-Y

Analysis of the structures of the above families of Fc-region binding polypeptides revealed the following general formulae for Fc-region binders:

Z₁—X₁—X₂—X₃—X₄—W—C—Z₂(SEQ ID NO:4); I.

wherein,

Z₁ is a polypeptide of at least 6 amino acids;

X₁ is G, H, N, R, or S;

X₂ is A, D, E, F, I, M, or S;

X₃ is A, I, L, M, or V;

X₄ is I, M, T, or V;

Z₂ is a polypeptide of at least one amino acid or is absent; and

Z₁ contains at least one cysteine residue such that formation of a disulfide bond with the invariant cysteine residue forms a cyclic peptide of 12 amino acids.

Z₁—X—W—Z₂—W—Z₃(SEQ ID NO:5) II.

wherein,

Z₁ is a polypeptide of at least one amino acid or is absent;

X is F or Y;

Z₂ is a tripeptide; and

Z₃ is a polypeptide of at least one amino acid; and

wherein at least two of the polypeptides Z₁, Z₂, and Z₃ contain a cysteine residue, such that formation of a disulfide bond between such cysteine residues forms a cyclic peptide of 7-12 amino acids.

In the foregoing formula II polypeptides, it is preferred that Z₂ has the formula (IIA):

X₁—X₂—X₃(IIA),

wherein,

X₁ is A, C, F, K, P, R, W, or Y;

X₂ is C, D, E, G, H, K, M, N, Q, R, S, T, V, or Y; and

X₃ is A, E, F, H, I, K, L, Q, R, S, T, V, or Y;

with the proviso that at most only one of X₁, X₂ and X₃ can be C. Preferably, where X₂ is C, then X₁ is Y. Most preferably, X₁ is C.

Z₁—W—Z₂—W—W—Z₃(SEQ ID NO:6); III.

wherein,

Z₁ is a polypeptide of at least one amino acid;

Z₂ is a tripeptide; and

Z₃ is a polypeptide of at least one amino acid;

wherein at least two of the polypeptides Z₁, Z₂, and Z₃ contain a cysteine residue, such that formation of a disulfide bond between such cysteine residues forms a cyclic peptide of 8-12 amino acids, with the proviso that where Z₁ contains a cysteine, then Z₂ does not contain a cysteine, and where Z₂ contains a cysteine, it is the middle residue of the tripeptide and Z₃ also contains a cysteine.

Preferably for the polypeptides of formula III, when Z₁ and Z₃ each contain a cysteine residue, the cysteine of Z₁ is adjacent the invariant tryptophan (W), the first amino acid of Z₂ is lysine and the second amino acid of Z₃ is apartic acid (D).

Z₁—P—X₁—W—X₂—C—X₃—X₄—X₅(SEQ ID NO:7); IV.

wherein, Z₁ is a polypeptide of at least one amino acid and includes a cysteine residue;

X₁ is A, E, R, S, or T;

X₂ is F, W, or Y;

X₃ is D, E, L, M, or Q;

X₄ is H, W, or Y;

X₅ is F or Y; and

wherein the cysteine residue in Z₁ and the cysteine residue between X₂ and X₃ form a cyclic peptide of 10-12 amino acids.

Synthesis of Fc Binding Peptide

Once isolated, the sequence of any individual binding peptide or the structure of any binding molecule can be analyzed, and the binder may be produced in any desired quantity using known methods. For example, the peptide binding molecules described herein, since their sequences are disclosed, may advantageously be produced by chemical synthesis followed by treatment under oxidizing conditions appropriate to obtain the native conformation, i.e., the correct disulfide bond linkages. Synthesis may be carried out by methodologies well known to those skilled in the art (see, Kelley et al. in Genetic Engineering Principles and Methods, (Setlow, J. K., ed.), Plenum Press, NY., (1990) vol. 12, pp. 1-19; Stewart et al., Solid-Phase Peptide Synthesis (1989), W. H. Freeman Co., San Francisco. The binding molecules of the present invention can be made either by chemical synthesis or by semisynthesis. The chemical synthesis or semisynthesis methods allow the possibility of non-natural amino acid residues to be incorporated.

Polypeptide binding molecules of the present invention are preferably prepared using solid phase peptide synthesis (Merrifield, J. Am. Chem. Soc., 85: 2149 (1963); Houghten, Proc. Natl. Acad. Sci. USA, 82: 5132 (1985)). Solid phase synthesis begins at the carboxy-terminus of the putative peptide by coupling a protected amino acid to a suitable resin, which reacts with the carboxy group of the C-terminal amino acid to form a bond that is readily cleaved later, such as a halomethyl resin, e.g., chloromethyl resin, bromomethyl resin, hydroxymethyl resin, aminomethyl resin, benzhydrylamine resin, or t-alkyloxycarbonyl-hydrazide resin. After removal of the α-amino protecting group with, for example, trifluoroacetic acid (TFA) in methylene chloride and neutralizing in, for example, TEA, the next cycle in the synthesis is ready to proceed. The remaining α-amino and, if necessary, side-chain-protected amino acids are then coupled sequentially in the desired order by condensation to obtain an intermediate compound connected to the resin. Alternatively, some amino acids may be coupled to one another forming an oligopeptide prior to addition of the oligopeptide to the growing solid phase polypeptide chain.

The condensation between two amino acids, or an amino acid and a peptide, or a peptide and a peptide can be carried out according to standard condensation methods such as azide method, mixed acid anhydride method, DCC (dicyclohexylcarbodiimide) method, active ester method (p-nitrophenyl ester method, BOP [benzotriazole-1-yl-oxy-tris (dimethylamino)phosphonium hexafluorophosphate] method, N-hydroxysuccinic acid imido ester method), and Woodward reagent K method.

Common to chemical synthesis of peptides is the protection of the reactive side-chain groups of the various amino acid moieties with suitable protecting groups at that site until the group is ultimately removed after the chain has been completely assembled. Also common is the protection of the α-amino group on an amino acid or a fragment while that entity reacts at the carboxyl group followed by the selective removal of the α-amino-protecting group to allow subsequent reaction to take place at that location. Accordingly, it is common that, as a step in the synthesis, an intermediate compound is produced which includes each of the amino acid residues located in the desired sequence in the peptide chain with various of these residues having side-chain protecting groups. These protecting groups are then commonly removed substantially at the same time so as to produce the desired resultant product following purification.

The typical protective groups for protecting the α- and ε-amino side chain groups are exemplified by benxyloxycarbonyl (Z), isonicotinyloxycarbonyl (iNOC), O-chlorobenzyloxycarbonyl [Z(NO₂)], p-methoxybenzyloxycarbonyl [Z(OMe)], t-butoxycarbonyl (Boc), t-amyioxycarbonyl (Aoc), isobomyloxycarbonyl, adamatyloxycarbonyl, 2-(4-biphenyl)-2-propyloxycarbonyl (Bpoc), 9-fluorenylmethoxycarbonyl (Fmoc), methylsulfonyiethoxycarbonyl (Msc), trifluoroacetyl, phthalyl, formyl, 2-nitrophenylsulphenyl (NPS), diphenylphosphinothioyl (Ppt), dimethylophosphinothioyl (Mpt), and the like.

As protective groups for the carboxy group there can be exemplified, for example, benzyl ester (OBzl), cyclohexyl ester (Chx), 4-nitrobenzyl ester (ONb), t-butyl ester (Obut), 4-pyridylmethyl ester (OPic), and the like. It is desirable that specific amino acids such as arginine, cysteine, and serine possessing a functional group other than amino and carboxyl groups are protected by a suitable protective group as occasion demands. For example, the guanidino group in arginine may be protected with nitro, p-toluenesulfonyl, benzyloxycarbonyl, adamantyloxycarbonyl, p-methoxybenzenesulfonyl, 4-methoxy-2,6-dimethylbenzenesulfonyl (Mds), 1,3,5-trimethylphenysulfonyl (Mts), and the like. The thiol group in cysteine may be protected with p-methoxybenzyl, triphenylmethyl, acetylaminomethyl ethylcarbamoyl, 4-methylbenzyl, 2,4,6-trimethy-benzyl (Tmb) etc., and the hydroxyl group in the serine can be protected with benzyl, t-butyl, acetyl, tetrahydropyranyl, etc.

After the desired amino acid sequence has been completed, the intermediate peptide is removed from the resin support by treatment with a reagent, such as liquid HF and one or more thio-containing scavengers, which not only cleaves the peptide from the resin, but also cleaves all the remaining side-chain protecting groups. Following HF cleavage, the protein sequence is washed with ether, transferred to a large volume of dilute acetic acid, and stirred at pH adjusted to about 8.0 with ammonium hydroxide. Upon pH adjustment, the polypeptide takes its desired conformational arrangement.

Polypeptides according to the invention may also be prepared commercially by companies providing peptide synthesis as a service (e.g., BACHEM Bioscience, Inc., King of Prussia, Pa.; Quality Controlled Biochemicals, Inc., Hopkinton, Mass.).

Use of the Binding Molecules in Detection and Purification

For detection of Fc-region polypeptides alone or present as a domain of a larger molecule (e.g., a natural or synthetic immunoglobulin or Fc-region-containing fragment thereof) in a solution such as whole blood, plasma, transgenic milk, eggs of transgenic chickens, conditioned media, or any solution or feed stream containing such Fc-region targets, a binding molecule according to the invention can be detectably labeled, e.g., radiolabeled or enzymatically labeled, then contacted with the solution, and thereafter formation of a complex between the binding molecule and the Fc-region polypeptide target can be detected. Alternatively, a phage binding molecule according to the invention, i.e., a recombinant phage displaying an Fc-region binding polypeptide on its surface, may form a complex with the Fc-region polypeptide that is detectable as a sediment in a reaction tube, which can be detected visually after settling or centrifugation.

Alternatively, a sandwich-type assay may be used, wherein an Fc-region binding molecule is immobilized on a solid support such as a plastic tube or well, or a chromatographic matrix such as sepharose beads, then the solution suspected of containing the Fc-region polypeptide target is contacted with the immobilized binding molecule, non-binding materials are washed away, and complexed Fc-region polypeptide is detected using a suitable detection reagent, such as a monoclonal antibody recognizing the Fc-region target, which reagent is detectable by some conventional means known in the art, including being detectably labeled, e.g., radiolabeled or labeled enzymatically, as with horseradish peroxidase, and the like.

The binding molecules according to this invention will be extremely useful for isolation of Fc-region polypeptides by affinity chromatography methods. Any conventional method of chromatography may be employed. Preferably, a binding molecule of the invention will be immobilized on a solid support suitable, e.g., for packing a chromatography column. The immobilized affinity ligand can then be loaded or contacted with a feed stream under conditions favorable to formation of binding molecule/Fc-region polypeptide complexes. Non-binding materials can be washed away, then the Fc-region polypeptides can be eluted by introducing solution conditions favoring dissociation of the binding complex.

Alternatively, batch chromatography can be carried out by mixing a solution containing the Fc-region polypeptide target and the binding molecule, then isolating complexes of the Fc-region polypeptide target and the binding molecules. For this type of separation, many methods are known. For example, the binding molecule can be immobilized on a solid support, then separated from the feed stream along with the Fc-region polypeptide target by filtration. Or the binding molecule may be modified with its own affinity tag, such as a polyHis tail (e.g., hexahistidine), biotin, myc tag, etc., which can be used to capture the binding molecule after complexes have formed, using the binding partner for the affinity tag, e.g., an immobilized metal affinity chromatography resin for capturing polyHis-tagged molecules, streptavidin for biotinylated molecules, etc. Once separated, the Fc-region polypeptide target can be released from the binding molecule under elution conditions and recovered in pure form.

It should be noted that although precise binding and release conditions were selected in obtaining the Fc-region amino acid-binding polypeptides disclosed herein, subsequent use in affinity purification may reveal more optimal binding and release conditions under which the same isolated affinity ligand will operate. Thus, it is not critical that the binding molecule, after isolation according to this invention, be always employed only at the binding and release conditions that led to its separation from the library.

Isolation of Fc-region binding molecules in accordance with this invention will be further illustrated below. The specific parameters included in the following examples are intended to illustrate the practice of the invention, and they are not presented to in any way limit the scope of the invention.

The phage libraries used in the present invention are constructed in derivatives of the filamentous phage M13. The displayed peptides are fused to the amino terminus of protein III through a linker peptide which contains the recognition site for Factor Xa. Factor Xa can cleave the displayed peptide from the phage without injuring the phage or reducing its infectivity.

Use of the Binding Molecules to Increase the Serum Half-life of a Compound

Another use for the Fc-binding polypeptides of the present invention is to increase the half-life and overall stability of a therapeutic or diagnostic compound that is administered to or enters the circulatory system of an individual. See, e.g., U.S. Pat. No. 5,116,944; EP-A2-395 918; WO 91/01743, incorporated herein in their entirety by reference. In such methods, an Fc-binding polypeptide described herein is used to link a therapeutic or diagnostic compound to an antibody found in the blood of an individual who will receive the therapeutic or diagnostic compound. In this embodiment, an Fc-binding polypeptide of the invention is linked, covalently or non-covalently, to a selected therapeutic or diagnostic compound at a site that keeps the Fc-binding polypeptide antibody binding site intact and therefore still capable of binding to an antibody molecule, without compromising the desired diagnostic or therapeutic activity. In this way, the Fc-binding polypeptide serves as a linker molecule to link the diagnostic/therapeutic compound of interest to an antibody circulating in the blood.

Linking a diagnostic or therapeutic compound to a circulating antibody using an Fc-binding polypeptide of the invention is expected to be particularly useful in increasing the circulating half-life and/or overall stability of compounds that are normally subject to an undesirably rapid rate of degradation or clearance from circulation. Increasing the half-life or overall stability of a compound in the circulatory system is likely to reduce the number and/or size of doses that must be administered to an individual to obtain a desired effect. Any suitable diagnostic compound may be linked to an antibody molecule in this manner, including, especially detectable labels, which may be a dye (such as fluorescein); radiolabels such as ¹³¹I or a technetium (Tc⁹⁹)-containing compound; enzymes (such as horseradish peroxidase); or a detectable metal (such as a paramagnetic ion). Any suitable therapeutic compound may be linked to an antibody molecule in this manner, including drugs, biopharmaceuticals, and any polypeptide of interest. Examples of such therapeutics suitable for linking to antibodies include but are not limited to receptor agonists or antagonists, specific binding compounds, enzyme inhibitors, metal chelators, molecular scavengers such as vitamin E, and the like. Of particular interest for this use are thrombin inhibitors, thrombolytics (such as tPA and urokinase), renin inhibitors, ACE inhibitors, selectin ligands, inhibitors of the coagulation cascade, complement regulatory molecules (such as DAF, CR1, CR2, C4 bp, factor H), serine proteases, GPIIb/IIIa antagonists, CRF antagonists, and the like.

### EXAMPLE 1

### The Isolation of Binding Molecules for Fc-region Amino Acid Peptides

The techniques described above were employed to isolate binding molecules for ligands for Fc-region amino acid peptides. Human IgG Fc fragment isolated from plasma (Calbiochem, cat.# 401104) was lightly biotinylated at a ratio of 2:1, moles biotin:Fc protein. The biotin-Fc was bound to non-porous streptavidin beads (Dynel). Prior to screening, libaries TN6/6, TN7/4, TN8/9, TN9/4, TN10/9, and TN12/1 were depleted of streptavidin, biotin and bead-binding phage by exposure to biotin-streptavidin beads. Next, the individual libraries were screened against the streptavidin-immobilized biotin-Fc-region target on beads and competition-eluted with an approximately ten-fold molar excess of Protein A. Pools were then made of the Protein A eluates such that TN12 and TN10 libraries were pooled, TN9 and TN8 libraries were pooled, and TN7 and TN6 libraries were pooled. The Protein A-eluted beads from each library were pooled in the same way and plated directly onto E. coli growth media to capture any tightly bound phage not eluted with Protein A. Protein A eluate library pairs and the bead-plated library pairs were then amplified and carried separately through two more rounds of screening with the biotin-Fc on beads.

Sequencing of recovered isolates has revealed 128 different Fc-region binding polypeptides thus far (SEQ ID NOS:14-143). Randomly chosen phage isolates from the third round of screening from each library pool were evaluated by phage ELISA on plates exhibiting streptavidin-biotinylated Fc, BSA, biotin, streptavidin-biotin, streptavidin-BSA, and on the polystyrene plate alone. The biotinylated Fc captured all IgG Fc isotypes. Of the randomly-chosen isolates, approximately 95% from each group, the sequences of which are disclosed herein, showed positive binding for the ELISA experiments. In addition, isolates were examined by ELISA signal under competition with various concentrations with soluble Fc-region polypeptide.

### EXAMPLE 2

### Binding Studies

Based on several criteria, i.e., high absorbance to various loading of immobilized Fc, effective response to soluble Fc competition, and either a representative binding motif or unique binding motif based on comparative sequence analysis, four phage isolates were chosen for further analysis to determine Fc binding constants by fluorescence anisotropy and to investigate various parameters that affect binding to determine optimal conditions useful for the affinity purification of Fc:

| | |
|---|---|
| Ac-GDDHMCVYTTWGELIWCDNHEPGPEGGGK-NH₂ ; | (SEQ ID NO:144, designated DX249) |
| Ac-AGPVDCKHHFWWCYWNGTPGPEGGGK-NH₂ ; | (SEQ ID NO:153, designated DX251) |
| Ac-GDDDHCYWFREWFNSECPHGEPGPEGGGK-NH₂ ; | (SEQ ID NO:154, designated DX252) |
| Ac-AGYYWCNYWGLCPDQGTPGPEGGGK-NH₂ ; | (SEQ ID NO:155, designated DX254) |

Peptides were synthesized by BACHEM Bioscience, Inc., (King of Prussia, Pa.), fluorescein labeled and purified by HPLC. Binding studies were performed using either a mixture of human plasma IgG isoforms (the Fc target) or the individual human plasma IgG isoforms: IgG1, IgG2, IgG3, or IgG4. All Fc target proteins were obtained from Calbiochem.

All measurements were performed in 384 well microplates with 20 nM fluorescein-labeled peptide and various concentrations of the IgG isoform in a volume of between 10 and 20 μl using a Tecan Polarion fluorescence polarization plate reader. All binding buffers included 0.01% (v/v) Tween 20 to prevent peptide adsorption to the microplate. The binding mixture was equilibrated for 10 minutes in the microplate at 30° C. prior to performing the measurement.

Cross-competition studies between peptides were performed using 20 nM fluorescein-labeled peptide and 1-2 μM IgG1, IgG2, IgG3, or IgG4 in the presence and absence of a maximum of 100 μM unlabeled peptide or 5 mg/ml protein A.

Dissociation constants that describe IgG isoform binding for Fc-region binding polypeptied DX249, DX252, and DX254 are listed in Table 1.

The data in Table 1 demonstrates that the peptides bind IgG in a pH-dependent manner. For instance, increasing the pH of the buffer from 5.7 to 7.4-9.3, increases the K_{D} of the interaction between DX249 or DX252 and the IgG isoforms. In contrast, DX254 does not appear to bind IgG1 or IgG2 in a pH-dependent manner. Additionally, the data in Table 1 demonstrate IgG subtype specificity, for example, DX249 binds to IgG1, IgG2, and IgG4 but not IgG3.

TABLE 1
Dissociation Constants (K_{D}) between IgG Isoforms and
Synthetic Peptides in Low Ionic Strength Buffers.

| | | K_{D} (μM)^{Φ} | | |
|---|---|---|---|---|
| Protein Target | pH^{#} | DX249 | DX252 | DX254 |
| IgG1 | 5.7 | 0.074 ± 0.053* | 0.1 ± 0.056 | 0.09 ± 0.09 |
| IgG2 | 5.7 | 0.068 ± 0.045 | >1 | >1 |
| IgG3 | 5.7 | >10 | 0.06 ± 0.1 | 0.3 ± 0.3 |
| IgG4 | 5.7 | 0.3 ± 0.3 | >10 | >10 |
| IgG1 | 7.4 | 0.4 ± 0.06 | 2.1 ± 2 | 1.8 ± 0.6 |
| IgG2 | 7.4 | 1.8 ± 0.6 | 3.4 ± 1.5 | 2 ± 0.7 |
| IgG3 | 7.4 | >10 | 2.3 ± 0.4 | >10 |
| IgG4 | 7.4 | 1.9 ± 0.4 | 3.2 ± 2.6 | 2.9 ± 1.6 |
| IgG1 | 9.3 | >10 | >10 | 0.4 ± 0.4 |
| IgG2 | 9.3 | >10 | >10 | 2 ± 2.7 |
| IgG3 | 9.3 | >10 | >10 | >10 |
| IgG4 | 9.3 | >10 | >10 | >10 |

^{#}Buffers used: 1) pH 5.7, 0.01 M Tris-acetate 2) pH 7.4, 0.01 M Tris-HCl 3) pH 9.3, 0.01 M sodium bicarbonate
^{Φ}The error in the K_{D} values is due to the standard error from the nonlinear regression as calculated by SigmaPlot.
*This particular measurement is the average of 5 separate determinations.

The data in Table 2 demonstrate that the K_{D} between DX249 and IgG1 at pH 5.7 increases approximately ten-fold on raising the sodium chloride concentration in the buffer from 0 to 100 mM.

TABLE 2
Effect of Sodium Chloride Concentration on the Dissociation
Constant (K_{D}) between IgG1 and Synthetic Peptides at pH 5.7.

| | K_{D} (μM)^{Φ} |
|---|---|
| [NaCl] (M) | DX249 |
| 0 | 0.074 ± 0.053 |
| 0.05 | 0.3 ± 0.3 |
| 0.1 | 0.97 ± 0.8 |
| 0.15 | 0.84 ± 0.7 |

^{#}Buffers used: 1) pH 5.7, 0.01 M Tris-acetate 2) pH 5.7, 0.01 M Tris-acetate, 0.05 M NaCl 3) pH 5.7, 0.01 M Tris-acetate, 0.1 M NaCl 4) pH 5.7, 0.01 M Tris-acetate, 0.15 M NaCl

The data in Table 3 demonstrate that all three peptides appear to bind the Fc IgG isoform mixture approximately the same, 2-5 μM, and the pH dependence for peptide binding to IgG1 is maintained in the presences of 150-200 mM NaCl for DX249 and DX252. There was no observed effect of including 15% ethylene glycol on DX249 binding to IgG1 (Table 3).

TABLE 3
Dissociation Constants (K_{D}) between IgG Isoforms and
Synthetic Polypeptides in “High” Ionic Strength Buffers.

| | | | K_{D} (μM)^{Φ} | | |
|---|---|---|---|---|---|
| Protein | pH | [NaCl] (M) | DX249 | DX252 | DX254 |
| Fc | 8.0 | 0.2 | 2.5 ± 0.5 | 5.1 ± 3 | 1.6 ± 0.6 |
| IgG1 | 5.7 | 0.15 | 0.84 ± 0.7 | | |
| IgG1 | 7.4 | 0.15 | 1.4 ± 0.4 | 1.9 ± 1.3 | 0.14 ± 0.14 |
| | | | 2.9 ± 0.9 | 2.9 ± 1.6 | 0.4 ± 0.2 |
| IgG1 | 9.3 | 0.2 | >10 | >10 | 0.6 ± 0.5 |
| IgG2 | 7.4 | 0.15 | 1.1 ± 0.3 | 3.6 ± 2.1 | 1.1 ± 0.3 |
| | | | | 3.0 ± 2.6 | |
| IgG3 | 7.4 | 0.15 | >10 | 2.7 ± 2 | 3.0 ± 1.1 |
| IgG4 | 7.4 | 0.15 | 1.9 ± 1.2 | >10 | >10 |
| IgG1 | 5.7 | 0 (15% EG) | 0.08 ± 0.2 | | |
| IgG1 | 7.4 | 0.15 (15% EG) | 3.6 ± 2.5 | | |

^{#} Buffers used: 1) pH 8.0, 0.05 M Tris-HCl, 0.2 M NaCl 2) pH 5.7, 0.01 M Tris-acetate, 0.15 M NaCl 3) pH 7.4, PBS 4) pH 9.3, .01 M sodium bicarbonate, 0.2 M NaCl 5) pH 5.7, 0.01 M Tris-acetate, 15% EG 6) pH 7.4 PBS,15% EG
EG: ethylene glycol

Table 4 demonstrates cross-competition studies between peptides DX249, DX251, DX252, and DX254, using the IgG isoforms. Results demonstrate that all of the peptides, with the possible exception of DX254, appear to bind to the same isoforms in the Fc mixture and to the same sites.

TABLE 4
Cross-Competition between Synthetic Peptides for Fc Binding.

Unlabeled Peptide

| Labeled Peptide | DX249 | DX251 | DX252 | DX254 |
|---|---|---|---|---|
| DX249 | + | + | + | + |
| DX252 | + | + | + | + |
| DX254 | − | − | − | + |

Table 5 demonstrates competition studies between DX249, DX252, or DX254 and Protein A for Fc binding. The data indicate that all of the peptides tested bind competitively with Protein A to IgG1, IgG2, and IgG4. In contrast, Protein A does not appear to compete with DX252 and possibly DX254 for binding to IgG3. This is consistent with the fact that Protein A is known not to bind human IgG3.

TABLE 5
Competition between Synthetic Peptides
and Protein A for FC Binding.

| Labeled Peptide | IgG1 | IgG2 | IgG3 | IgG4 |
|---|---|---|---|---|
| DX249 | + | + | NB | + |
| DX252 | + | + | − | NB |
| DX254 | + | + | ? | NB |

NB: no detected binding.
?: partial decrease in anisotropy with added competitor.

The above data indicate a possible affinity purification procedure whereby the Fc is bound to the peptide column at low pH, washed with high salt, and eluted with a low salt, high pH buffer.

### EXAMPLE 3

### Additional Binding Studies

Peptides DX249, DX250 and DX253 were modified with acetylated N-terminal peptides and a C-terminal amine-functional linker to facilitate immobilization on an N-hydroxysuccinimide sepharose chromatography resin (Pharmacia). Additional binding studies were performed with the following peptides:

Ac-GDDHMCVYTTWGELIWCDNHEPGPEGGGK-NH₂ (SEQ ID NO:144, flouresceinated at the C-terminus and designated DX249);

Ac-GDRRACSRDWSGALVWCAGHEPGPEGGGK-NH₂ (SEQ ID NO:145, flouresceinated at the C-terminus and designated DX301); and

Ac-AGKYWCSFWGLQCKTGTPGPEGGGK-NH₂ (SEQ ID NO:146, flouresceinated at the C-terminus and designated DX300).

Peptides were synthesized, fluorescein-labeled and HPLC purified as described above. Binding studies were performed using either a mixture of human plasma IgG isoforms (the Fc protein) or the individual human plasma IgG isoforms: IgG1, IgG2, IgG3, or IgG4.

Measurements were performed in 384-well microplates with 2 nM fluorescein-labeled peptide and a varied concentration of the IgG isoform target in a volume of 10 μl using a Tecan Polarion fluorescence polarization plate reader. The Fc binding buffer was 50 mM Tris-HCl, 200 mM NaCl, pH 8.0, 0.01% Tween 20. The IgG binding buffer was PBS with 0.01% Tween 20. The binding mixtures were equilibrated for 10 minutes in the microplate at 30° C. prior to performing the measurements.

Cross-competition studies between peptides were performed using 20 nM fluorescein-labeled peptide and 6 μM Fc (with DX276), 11.5 μM Fc (with DX301) or 2 μM IgG3 (with DX300) in the presence or absence of a maximum of 100 μM unlabeled peptide.

The data in Table 6 demonstrate that peptide DX300 and peptide DX301 both exhibit IgG isoform specificity. DX300 binds only IgG3, whereas DX301 binds IgG1, IgG2, and IgG4, but not IgG3.

TABLE 6
Dissociation Constants for DX300 and DX301

| Protein | DX300 K_{D} (μM) | DX301 K_{D} (μM) |
|---|---|---|
| Fc | No binding | 6.8 ± 4.3 |
| IgG1 | No binding | 3.6 ± 2.6 |
| IgG2 | No binding | 2.3 ± 0.8 |
| IgG3 | 4.1 ± 4.6 | No binding |
| IgG4 | No binding | 9.7 ± 13 |

### EXAMPLE 4

### Additional Cross-Competition Studies

Cross-competition studies were performed, testing the labeled Fc binding of peptides DX276, DX300 and DX301 against the following soluble Fc-region binding peptides:

| | |
|---|---|
| Ac-GDDHMCVYTTWGELIWCDNHEPGPEGGGK-NH₂ ; | (SEQ ID NO:144, designated DX 249) |
| Ac-AGKYWCSFWGLQCKTGTPGPEGGGK-NH₂ ; | (SEQ ID NO:146, designated DX250) |
| Ac-AGPVDCKHHFWWCYWNGTPGPEGGGK-NH₂ ; | (SEQ ID NO:153, designated DX 251) |
| Ac-GDDDHCYWFREWFNSECPHGEPGPEGGGK-NH₂ ; | (SEQ ID NO:154, designated DX 252) |
| Ac-GDRRACSRDWSGALVWCAGHEPGPEGGGK-NH₂ ; and | (SEQ ID NO:145, designated DX 253) |
| Ac-AGYYWCNYWGLCPDQGTPGPEGGGK-NH₂ ; | (SEQ ID NO:155, designated DX254) |

Table 7 shows the results of cross-competition experiments with the labeled peptide and protein target as described above and with soluble competitors DX249, DX250, DX251, DX252, DX253, and DX254.

As can be seen from the data in Table 7, peptide DX249 does not compete with DX300 and therefore does not bind to IgG3.

Peptide DX250 competes with its labeled peptide counterpart, i.e., DX300, but also competes with DX276 and DX301, suggesting that it may weakly bind IgG1 and/or IgG2 or IgG4. Peptides DX251 and DX252 only compete with DX276.

Peptide DX253 does not compete with DX300 and therefore does not bind to IgG3.

Finally, peptide DX254 appears to compete with all the fluorescein labeled peptides examined.

TABLE 7
Cross-Competition

| | Pro- | | | | | | |
|---|---|---|---|---|---|---|---|
| | tein | | | | | | |
| Labeled | Tar- | Unlabeled Peptide | | | | | |
| Peptide | get | DX249 | DX250 | DX251 | DX252 | DX253 | DX254 |
| DX276 | Fc | + | + | + | + | + | + |
| DX300 | IgG3 | − | + | − | − | − | + |
| DX301 | Fc | + | + | − | − | + | + |

### EXAMPLE 5

### Dissociation Constants

Dissociation constants were determined for the following peptides, which were prepared using the Fc-region binding peptides of SEQ ID NOS: 57, 58, 108, 115, 124, and 143, respectively:

Ac-AGSYWCKIWDVCPQSPGPEGGGK-NH₂ (SEQ ID NO:147, designated DX392);

Ac-AGKYWCNLWGVCPANPGPEGGGK-NH₂ (SEQ ID NO:148, designated DX395);

Ac-AGTYWCTFWELPCDPAPGPEGGGK-NH₂ (SEQ ID NO:149, designated DX404);

Ac-AGPHNCDDHYWYCKWFPGPEGGGK-NH₂ (SEQ ID NO:150, designated DX389);

Ac-AGAATCSTSYWYYQWFCTDSPGPEGGGK-NH₂ (SEQ ID NO:151, designated DX398); and

Ac-AGYWYCWFPDRPECPLYPGPEGGGK-NH₂ (SEQ ID NO:152, designated DX413).

Peptides were synthesized by BACHEM and then Oregon Green labeled and HPLC purified. Binding studies were performed using human plasma IgG isoforms: IgG1, IgG2, IgG3, and IgG4, obtained from Calbiochem.

Binding studies were carried out at either pH 4.0, 7.5, or 9.5, with or without salt in the following buffers:

1) 10 mM Sodium Citrate, 0.01% Tween 20, pH 4.0;

2) 10 mM Sodium Citrate, 500 mM Sodium Chloride, 0.01% Tween 20, pH 4.0;

3) 10 mM Tris-HCl, 0.01% Tween 20, pH 7.5;

4) 10 nM Tris-HCl, 500 mM Sodium Chloride, 0.01% Tween 20, pH 7.5;

5) 10 mM Sodium Bicarbonate, 0.01% Tween 20, pH 9.5;

6) 10 mM Sodium Bicarbonate, 500 mM Sodium Chloride, 0.01% Tween 20, pH 9.5; or

7) TBS, 0.01% Tween 20, pH 7.5.

Results of the binding studies are shown in Table 8.

TABLE 8
Summary of K_{D} values for the IgG binding Oregon Green Labeled Peptides

| | | K_{D} (μM) | | | | | | |
|---|---|---|---|---|---|---|---|---|
| | IgG | pH 4.0 − | pH 4.0 + | pH 7.5 − | pH 7.5 + | pH 9.5 − | pH 9.5 + | |
| Peptide | isoform | salt | salt | salt | salt | salt | salt | TBS |
| DX389 | IgG1 | nb* | nb | nb | nb | nb | nb | nb |
| | IgG2 | nb | nb | nb | binds^{Φ} | nb | nb | nb |
| | IgG3 | 2.5 ± 1.0 | 1.8 ± 1.4 | nb | binds | nb | nb | nb |
| | IgG4 | nb | nb | nb | nb | nb | nb | nb |
| DX392 | IgG1 | nb | nb | nb | nb | nb | nb | nb |
| | IgG2 | nb | nb | nb | nb | nb | nb | nb |
| | IgG3 | 0.32 ± 0.08 | 0.6 ± 0.2 | nb | binds | nb | binds | nb |
| | IgG4 | nb | nb | nb | nb | nb | nb | nb |
| DX395 | IgG1 | nb | nb | nb | nb | nb | nb | nb |
| | IgG2 | nb | nb | nb | nb | nb | nb | nb |
| | IgG3 | 1.0 ± 0.26 | 1.8 ± 1 | nb | 1.9 ± 0.9 | nb | binds | nb |
| | IgG4 | nb | nb | binds | nb | nb | nb | nb |
| DX398 | IgG1 | 2.4 ± 3.3 | nb | 4.6 ± 1.2 | nb | nb | nb | nb |
| | IgG2 | 1.8 ± 1.2 | nb | nb | nb | binds | nb | nb |
| | IgG3 | 0.02 ± 1.0 | 0.04 ± 0.01 | nb | 0.3 ± 0.03 | binds | 0.3 ± 0.1 | binds |
| | IgG4 | 1.6 ± 1.5 | nb | 3.5 ± 0.8 | nb | nb | nb | nb |
| DX404 | IgG1 | 1.5 ± 0.8 | 2.0 ± 1.7 | 8.8 ± 4.0 | nb | nb | nb | nd |
| | IgG2 | 1 ± 0.4 | 2.0 ± 1 | 8.6 ± 3.5 | nb | nb | nb | nd |
| | IgG3 | 0.01 ± 0.01 | 0.20 ± 0.06 | 11 ± 5.4 | 3.7 ± 0.8 | nb | binds | nd |
| | IgG4 | nb | nb | nb | nb | nb | nb | nd^{#} |
| DX413 | IgG1 | nb | nb | nb | nb | nb | nb | nd |
| | IgG2 | nb | nb | nb | nb | nb | nb | nd |
| | IgG3 | 0.84 ± 0.08 | 1.1 ± 0.2 | nb | nb | nb | nb | nd |
| | IgG4 | nb | nb | nb | nb | nb | nb | nd |

*nb: no significant binding observed
^{Φ}binds: peptide appears to bind but the signal change could not be fit to obtain a reliable estimate of the K_{D.} The K_{D} is estimated to be greater than 10 μM.
^{#}nd: not determined.

The results shown in Table 8 demonstrate that DX389 specifically binds IgG3 at pH 4.0 in either the presence or absence of salt with moderate affinity (K_{D}≅2 μM). This interaction was not 10 observed in the presence or absence of salt either at pH 7.5 or 9.5.

Peptide DX392 bound IgG3 specifically at pH 4.0 both in the presence and absence of salt and with a high affinity (K_{D}≅0.3-0.6 μM). This interaction was lower at pH 7.5 and pH 9.5 in the presence of salt and was not observed at either pH in the absence of salt.

Peptide DX395 bound IgG3 specifically at pH 4.0 in either the presence or absence of salt at moderate affinity (K_{D}≅1-2 μM). The affinity was approximately the same (K_{D}≅1.9 μM) in the presence of salt. This interaction was diminished at pH 9.5 in the presence of salt and was not observed at pH 7.5 or 9.5 in the absence of salt.

Peptide DX398 bound all four IgG isoforms at pH 4.0 in the absence of salt with moderate affinity (K_{D}≅2 μM) for IgG1, IgG2, and IgG4, and high affinity (K_{D}≅0.02 μM) for IgG3. At pH 4.0 in the presence of salt, peptide DX398 maintained a high affinity for IgG3 but did not interact with IgG, IgG2, or IgG4.

At pH 7.5, DX398 bound IgG1 and IgG4 only in the absence of salt and in the presence of salt, only bound IgG3. At pH 9.5, this peptide only bound IgG3 and the interaction was favored by increasing ionic strength.

Peptide DX404 bound IgG1 and IgG2 at pH 4.0 in the presence or absence of salt with moderate affinity (K_{D}≅2 μM) and had a higher affinity for IgG3 (K_{D}≅0.01 μM). In the presence of salt, the affinity for IgG3 increased to 0.2 μM. The affinity for IgG1 and IgG2 was reduced at pH 7.5 in the absence of salt and not observed in the presence of salt or at pH 9.5. IgG3 binding at pH 7.5 and 9.5 was favored in the presence of salt.

Peptide DX413 bound only to IgG3 at pH 4.0 in the presence or absence of salt with moderate affinity (K_{D}≅1.0 μM).

The data in Table 8 indicate that the peptides bind IgG with varying isoform specificities in a pH and salt-dependent manner. In general, the peptides in Table 8 can be grouped into two “classes” based on their specificity and mode of interaction:

Class 1 includes DX389, DX392, DX395 and DX413. Essentially these peptides all appear to exhibit primary specificity for IgG3. In addition, the interaction appears to be favored by low pH and high ionic strength. Binding is weakest at high pH and low salt.

Class 2 includes DX398. This peptide exhibits isoform specificity that is alterable by ionic strength. At low pH in the absence of salt, this peptide binds all IgG isoforms but in the presence of salt, it only binds IgG3 with very high affinity (K_{D}0.04-0.3 μM) at pH 4.0, 7.5, and 9.5 (See Table 8). DX404 is similar to DX398, however this peptide, unlike DX398, does not exhibit the salt-dependent IgG3 specificity at pH 4.0 but does exhibit salt-dependent IgG3 specificity at pH 7.5 and 9.5.

In addition, this study presents the first detailed demonstration for the use of Oregon Green as a fluorescent probe in anisotropy measurements. This probe exhibits photophysical parameters similar to fluorescein and offers the advantage of a wider pH range over which it is fluorescent. This increase in pH range is due to the lower pKa of the carboxylic acid group found on both Oregon Green and fluorescein (pKa=4.7 for Oregon Green vs. 6.4 for fluorescein).

The fluorescence anisotropy data above indicate binding characteristics of several of the disclosed Fc-region binding polypeptides for mixed human Fc and various IgG Fc isoforms. The pH-and NaCl-dependent studies indicate possible schemes for binding and elution conditions. The data indicate that a possible affinity purification scheme will involve binding of the peptides to Fc in a low pH buffer followed by a high salt wash and elution with a low salt, high pH buffer.

### EXAMPLE 6

### Batch-binding Evaluation

Batch-binding studies were carried out by incubating 10 μl aliquots (packed bed volume) of selected immobilized DX peptides (affinity media) with 100 ∞l of a 0.5 mg/ml (50 μg per 10 μl resin or 5.0 mg/ml resin) solution of each of the following targets: human IgG1, IgG2, IgG3, and IgG4 (all human subtypes derived from myeloma patients, Calbiochem, catalog nos. 400120, 400122, 400124, and 400126, respectively). In addition, the following targets, all unfractionated IgGs, were also evaluated with selected immobilized peptides for species specificity characterization in a similar format: human IgG, goat IgG, bovine IgG, and mouse IgG (Sigma, product nos. I4506, I5256, I5506, and I15381, respectively). In addition, 10 μl aliquots of hydrolyzed NHS-activated Sepharose 4 Fast Flow (Amersham Pharmacia Biotech, product no. 17-0906-01) and immobilized recombinant Protein A (RepliGen Corp., product no. IPA-400) were also assayed as negative and positive controls. IgG targets were diluted into 10 mM phosphate buffer containing 150 mM NaCl and 0.01% Tween 20, pH 7.2 (PBST) or 50 mM Tris buffer containing, 150 mM NaCl and 0.01% Tween-20, pH 7.5 (TBST). Batch (static) binding and elution studies were conducted in a 96-well assay filterplate (Millipore MultiScreen-HV, with 0.45 μm Durapore membrane, product no. MAHVN4510). The target was allowed to incubate with the resin for 1-1.5 hours with shaking at room temperature. Unbound target was collected by centrifugation of samples from the assay plate directly into a low-UV absorbing 96-well collection plate (Costar, product no. 3635 or Greiner, product no. 655801). The unbound target was then quantitated by reading the absorbance at 280 nm (normalized to 1 cm pathlength, 1.4 mg/ml A280 extinction coefficient) of the collection plate on a microplate spectrophotometer (Molecular Devices Corp., SPECTRAmax PLUS) against a buffer blank.

The percentage bound target was determined by subtracting the mass determined as unbound from the corresponding condition's control values (control non-filtered added directly to the collection plate and/or the negative control). The assay plate was washed (3×) with 200 μl of binding buffer by vacuum filtration followed by addition of 100 μl of various elution conditions (e.g.: 0.1 M sodium bicarbonate buffer, pH 8.5 and/or 0.1 M sodium citrate buffer, pH 4.0). After a 30-60 minute incubation with shaking at room temperature, the eluted target was collected by centrifugation of the assay plate into another collection plate and quantitated in a similar manner. Percent elution of total load was then determined to characterize elution recovery efficiency under various conditions. The assay plate was then washed (3×) with 200 μl of binding buffer by vacuum filtration followed by addition of a resin cleaning condition (e.g., 0.1M CAPS buffer (3-[Cyclohexylamino]-1-propane-sulfonic acid), pH 11.5, to regenerate the resin for re-use. Percentage mass balance determination was calculated to be 90-100% on average.

TABLE 9
% IgG Capture - Batch Binding: Human IgG Subtype Specificity

| | Human IgG1 | Human IgG2 | Human IgG3 | Human IgG4 |
|---|---|---|---|---|
| Prot. A | 95 | 92 | 1 | 96 |
| DX249 | 91 | 92 | 0 | 82 |
| DX253 | 58 | 56 | 7 | 33 |
| DX398 | 6 | 0 | 79 | 2 |
| DX404 | 38 | 11 | 74 | 10 |
| DX392 | 13 | 15 | 76 | 9 |
| DX395 | 21 | 2 | 85 | 4 |
| DX252 | 34 | 24 | 35 | 6 |
| DX254 | 28 | 16 | 36 | 6 |
| DX389 | 4 | 11 | 26 | 3 |
| DX251 | 6 | 14 | 5 | 1 |

TABLE 10
Batch Binding (bdg) and Elution (elu) of
Human IgG (% of Total Load)

| | Human IgG1 | | | Human IgG2 | | | Human IgG3 | | | Human IgG4 | | |
|---|---|---|---|---|---|---|---|---|---|---|---|---|
| | bdg | elu pH | | bdg | elu pH | | bdg | elu pH | | | elu pH | |
| | % | 8.5 | 4.0 | % | 8.5 | 4.0 | % | 8.5 | 4.0 | % | 8.5 | 4.0 |
| Prot. A | 93 | 2 | 79 | 93 | 8 | 75 | 5 | 0 | 0 | 92 | 2 | 95 |
| DX249 | 91 | 63 | 51 | 92 | 54 | 63 | 3 | 0 | 0 | 82 | 66 | 54 |
| DX252 | 37 | 19 | 30 | 37 | 8 | 10 | 44 | 8 | 5 | 14 | 4 | N/ |
| | | | | | | | | | | | | A |
| DX253 | 56 | 44 | 14 | 63 | 32 | 12 | 15 | 0 | 5 | 40 | 22 | N/ |
| | | | | | | | | | | | | A |

TABLE 11
% IgG Capture - Batch Binding: Species Specificity

| | Human IgG | Goat IgG | Bovine IgG | Mouse IgG |
|---|---|---|---|---|
| Prot. A | 86 | 23 | 72 | 77 |
| DX249 | 84 | 0 | 0 | 0 |
| DX253 | 54 | 0 | 0 | 2 |
| DX398 | 9 | 0 | 2 | 4 |
| DX404 | 30 | 0 | 0 | 2 |
| DX392 | 4 | 0 | 0 | 0 |
| DX395 | 19 | 0 | 0 | 0 |
| DX252 | 20 | 0 | 0 | 1 |
| DX254 | 16 | 0 | 0 | 2 |
| DX389 | 4 | 0 | 0 | 0 |
| DX251 | 4 | 0 | 0 | 4 |

The results in Table 9 demonstrate that affinity media DX249 and DX253 exhibited similar batch-binding properties to Protein A, indicating that they may interact with the Fc region of human immunoglobulins in a similar manner. The other affinity media exhibited significantly different binding properties, including significant binding affinity to the human IgG3 isotype, to which Protein A does not bind. hIgG target elution was also demonstrated for selected immobilized DX peptides (affinity media) relative to Protein A. Percentage elution of total hIgG load (Table 10) showed affinity media DX249 and DX253 to elute at both pH 4.0 and 8.5 compared to Protein A, which elutes at acid pH range (pH 4.0). In addition, the affinity media tested showed no significant binding to goat, bovine and mouse IgG relative to Protein A (see, Table 11). Protein A showed a moderately high binding affinity to the goat, bovine and mouse immunoglobulins tested.

### EXAMPLE 7

### Chromatographic Performance of Fc-binding Affinity Media

Binding studies under dynamic conditions on mini-chromatography columns packed with selected immobilized DX peptides (affinity media) showed similar binding profiles to those found in the batch binding studies above. Thus, 250 μg of target (hIgG1, hIgG2, hIgG3, hIgG4, goat IgG and bovine IgG) at 0.250 mg/ml in PBST, pH 7.2 or TBST, pH 7.3 was loaded onto a 350 μl column (3×50 mm) run at 0.200 ml/min. flowrate (170 cm/hr). Flowthrough, elution and cleaning fractions were collected and assayed for total protein recovery by size-exclusion HPLC. Percentage mass balance was also determined to be 90-100% on average. Percent binding efficiency was generally higher than seen in batch measurements, so that affinity media DX249 and DX253 showed binding efficiencies similar to that of Protein A for hIgG1, hIgG2 and hIgG4 (all >90%). Affinity media DX404 also showed high binding efficiency specifically for hIgG3. In addition, the affinity media tested showed no significant affinity towards goat or bovine IgG. Protein A again showed moderate to high binding efficiency to both goat and bovine IgG respectively.

In another study, based on the batch-binding studies, three affinity media, DX249, DX253 and DX252 were selected for firther evaluation in a similar chromatographic system. For these experiments, 350 μl columns (3×50 mm) were packed with each media. The columns were tested with human Fc fragments or human IgG1 (Calbiochem, 401104 and 400120 respectively) spiked into PBST, pH 7.2 at a concentration of 0.5 g/l. 65 μg of Fc was loaded onto each column. After loading, the columns were washed with PBST, pH 7.2, eluted with 30 mM H₃PO₄, 150 mM NaCl, pH 2.0 and cleaned with 0.1M CAPS, pH 11.5 buffer (3-[Cyclohexylamino]-1-propane-sulfonic acid). Fractions collected were immediately neutralized to pH ˜7.2 with 2.0 M Tris, pH 8.0. All column operations were conducted at 200 μl/min (170 cm/hr).

Results demonstrate that affinity media DX249 and DX253 exhibit quantitative binding of both Fc and IgG1 (capture efficiency ≧92%). Affinity media DX252 showed lower capture efficiency (70 and 80% respectively), significantly better than it performed in batch-binding studies. This is not unexpected since the chromatographic system will have a greater number of theoretical equilibrium stages than the batch binding system, which should lead to higher capture efficiencies. Thus, affinity media DX253 and DX252 showed ≧85% elution recovery of bound and ≧89% purity of monomeric protein by size-exclusion HPLC (SEC-HPLC).

### EXAMPLE 8

### Capture of Human Fc Out of Tobacco Extract

The ability of affinity media based on DX253 to capture human Fc and human IgG1 out of tobacco extract was evaluated in separate experiments. The tobacco extract used was a representative concentration blank lot (CropTech, lot NV100-136). Human Fc or IgG1 was spiked into the tobacco extract at 50 μg/ml. 100 μg (2 ml) was loaded onto a 350 μl column packed the DX253 media at 200 μl/min. (170 cm/hr.). Prior to loading, the column had been equilibrated with 50 mM Tris buffer containing 150 mM NaCl and 0.01% Tween 20, pH 7.3 (TBST, pH 7.3). After loading, the column was washed with 50 mM Tris buffer containing 1M NaCl, pH 7.3 followed by elution with 30 mM H₃PO₄, 150 mM NaCl, pH 2.0. The column was then cleaned with 0.1 M CAPS, pH 11.5 and separately with 30% isopropanol, 5% acetic acid in water, ˜pH 2.7. Collected fractions were neutralized immediately with 2M Tris, pH 8, prior to analysis by size exclusion HPLC. Results demonstrate % recovery of 91 and 76% of total load and % purity of 79% and 89% monomeric protein for both Fc and IgG1 from tobacco extract, respectively.

### EXAMPLE 9

### Capture of Human IgG1 from Cell Culture Supernatant

The ability of affinity media DX249 to capture human IgG1 out of conditioned CHO (Chinese Hampster ovary) cell culture supernatant containing 5% fetal bovine serum was evaluated. Human IgG1 (93% monomer) was spiked into conditioned CHO media to 50 μg/ml and 5 ml (250 μg total) was loaded onto a 350 μl mini-column as described above. The column was washed to baseline with PBST, pH 7.2 and the flowthrough fraction collected. Bound IgG was eluted with 0.1M sodium bicarbonate buffer, pH 9.0, and the column cleaned with 0.1M CAPS, pH 11.5. The eluted and clean fractions were quickly neutralized to ˜pH 7.2 and assayed for mass recovery by SEC-HPLC. Thus, an 80% recovery (98% monomer) was obtained in the eluted fraction. The column load, flowtbrough, elution and cleaning fractions were further analyzed by electrophoresis (SDS-PAGE), Coomassie stain and Western analysis for the presence of human and bovine IgG. This analysis showed that affinity media DX249 specifically captured hIgG1 from conditioned CHO media containing 5% fetal bovine serum. The load showed the presence of both human IgG and bovine IgG. The flowthrough fraction showed the presence of bovine IgG with no detectable human IgG. The elution fraction showed the presence of human IgG serum with no apparent bovine IgG contamination.

These results demonstrate that DX249 and peptides of like specificity according to the invention can be used to quantitatively isolate human Fc-region polypeptides from culture media or other solutions that contain both human and other mammalian immunoglobulins.

### EXAMPLE 10

### Capture of Human IgG4 from Goat IgG in Buffer

The ability of affinity media DX249 to capture human IgG4 from goat IgG (5-fold excess) in buffer was also evaluated. 0.250 mg of hIgG4 (99.5% monomer) was added to 1.0 mg of goat IgG in a total volume of 1.0 ml of PBST, pH 7.2. Thus, 1.0 ml was loaded onto a 350 ul mini-column as described above. The column was washed to baseline with PBST, pH 7.2 and the flowthrough fraction collected. Bound IgG was eluted with 0.1M sodium bicarbonate buffer, pH 9.0 and the column cleaned with 0.1M CAPS, pH 11.5. The eluted and clean fractions were quickly neutralized to pH˜7.2 and assayed for mass recovery by SEC-HPLC. Thus, an 87% theoretical load (assumed to be human IgG4) recovery (99.5% monomer) was obtained in the eluted fraction. Likewise, a 102% theoretical load (assumed to be goat IgG) recovery was obtained in the flowthrough fraction. The column load, flowthrough, elution and cleaning fractions were further analyzed by electrophoresis (SDS-PAGE), Coomassie stain. This analysis showed that affinity media DX249 specifically captured hIgG4 from goat IgG at 5-fold excess in buffer. The load showed the presence of both human IgG and goat IgG. The flowthrough fraction showed the presence of goat IgG with no human IgG4. The elution fraction showed the presence of human IgG4 with no apparent goat IgG contamination. The cleaning fraction showed no protein present.

### EXAMPLE 11

### Batch Binding and Elution of Human IgG Subtypes

DX596 (SEQ ID NO:157), DX597 (SEQ ID NO: 158) and DX1070 (SEQ ID NO:159) were immobilized and tested in filterplate format (as previously described) for both species specificity and human IgG subtype binding and elution relative to Protein A and DX249 and DX253 (SEQ ID NOs:144 and 145). The polypeptides synthesized for these tests are set forth in Table 12, below.

DX596 contains two lysine residues within the selected sequence which were synthesized using the orthogonal protecting group, ivDde, to prevent coupling within the selected sequence (the likely binding site). Once the C-terminal lysine was coupled to the resin, the ivDde protecting groups were removed with 2% hydrazine in DMF. A derivative polypeptide (designated DX1071) was synthesized in which these two internal lysines were substituted with arginine and alanine to determine if it was possible to eliminate the need for synthesis with the iVDde protecting group and eliminating the de-protecting step. In addition, a second derivative (designated DX1072) was synthesized with the arginine-alanine substitution and also a substitution of the adjacent serine residue with histidine. These two residue-substituted derivatives of DX596 (DX1071 and DX1072) were also immobilized and tested in filterplate format in a similar manner. All DX-peptides were immobilized to an average ligand density of 1.8 μM/ml, as determined by quantitative amino acid analysis.

TABLE 12

Peptide Sequences

| | | SEQ |
|---|---|---|
| | | ID |
| DX-number | Amino Acid Sequence | NO: |
| DX249 | Ac-GDDHMCVYTTWGELIWCDNHEPGPEGGGK-NH₂ | 144 |
| DX253 | Ac-GDRRACSRDWSGALVWCAGHEPGPEGGGK-NH₂ | 145 |
| DX1070 | Ac-GDWGECTVTSYGELIWCGGLEPGPEGGGK-NH₂ | 159 |
| DX597 | Ac-GDSWNCAFHHNEMVWCDDGGTPGPEGGGK-NH₂ | 158 |
| DX596 | Ac-GDNPMCWKKSWWEDAYCINHEPGPEGGGK-NH₂ | 157 |
| DX1071 | Ac-GDNPMCWRASWWEDAYCINHEPGPEGGGK-NH₂ | 160 |
| DX1072 | Ac-GDNPMCWRAHWWEDAYCINHEPGPEGGGK-NH₂ | 161 |

In the foregoing table, Ac-denotes N-terminal acetylation, —NH₂ denotes C-terminal amidation.

The results, set forth in Table 13, below, demonstrate that affinity media using polypeptides DX596, DX597, and DX1070 exhibited batch binding properties similar to Protein A, DX249 and DX253 as previously described. IgG is not eluted from Protein A at high pH, whereas DX249, DX253, DX596 and DX597 showed efficient elution at both low pH (pH 2.5 to 4.5) and high pH (pH 9.0 and 9.5). No ligand (including Prot. A) showed good elution at pH 5.5. DX1070 showed only partial elution of human IgG at pH 3.0. For DX1070, a significant amount of non-pH 3.0 eluted human IgG was recovered in a cleaning step with 3M guanidine-HCl, pH 7.2. DX1071, the substitutional derivative of DX596, showed no significant binding of human IgG. DX1072 showed a binding profile similar to Prot. A, DX249, DX253, DX596, DX597 and DX1070 (both human IgG subtype and species specificity), although binding efficiency was much lower.

TABLE 13
Batch Binding and Elution of Human IgG (% of Total Load)

| | Bound | pH 2.5 | pH 3.0 | pH 3.5 | pH 4.5 | pH 5.5 | pH 9.0 | pH 9.5 |
|---|---|---|---|---|---|---|---|---|
| hIgG1 | | | | | | | | |
| Prot. A | 96 | 80 | 80 | 86 | 29 | 3 | 3 | 3 |
| DX249 | 88 | 65 | 69 | 69 | 37 | 1 | 65 | 68 |
| DX253 | 86 | 78 | 75 | 54 | 55 | 8 | 44 | 45 |
| DX596 | 87 | 66 | N/A | 78 | 61 | 3 | 62 | 58 |
| DX597 | 93 | 76 | N/A | 81 | 39 | 1 | 52 | 57 |
| DX1070 | 87 | N/A | 22 | N/A | N/A | N/A | N/A | N/A |
| DX1071 | 7 | N/A | 4 | N/A | N/A | N/A | N/A | N/A |
| DX1072 | 31 | N/A | 27 | N/A | N/A | N/A | N/A | N/A |
| hIgG2 | | | | | | | | |
| Prot. A | 85 | 75 | 75 | 76 | 65 | 22 | 13 | 18 |
| DX249 | 78 | 71 | 73 | 63 | 39 | 1 | 60 | 67 |
| DX253 | 81 | 78 | 69 | 55 | 31 | 0 | 42 | 47 |
| DX596 | 81 | 71 | N/A | 92 | 44 | 3 | 58 | 64 |
| DX597 | 90 | 82 | N/A | 80 | 38 | 1 | 51 | 60 |
| DX1070 | 82 | N/A | 42 | N/A | N/A | N/A | N/A | N/A |
| DX1071 | 2 | N/A | 7 | N/A | N/A | N/A | N/A | N/A |
| DX1072 | 21 | N/A | 21 | N/A | N/A | N/A | N/A | N/A |
| hIgG3 | | | | | | | | |
| Prot. A | 0 | 0 | 0 | 0 | 0 | 0 | 0 | 0 |
| DX249 | 0 | 0 | 0 | 0 | 0 | 0 | 0 | 0 |
| DX253 | 0 | 0 | 0 | 0 | 0 | 0 | 0 | 0 |
| DX596 | 0 | 0 | N/A | 0 | 0 | 0 | 0 | 0 |
| DX597 | 0 | 0 | N/A | 0 | 0 | 0 | 0 | 0 |
| DX1070 | 0 | N/A | 0 | N/A | N/A | N/A | N/A | N/A |
| DX1071 | 0 | N/A | 0 | N/A | N/A | N/A | N/A | N/A |
| DX1072 | 0 | N/A | 0 | N/A | N/A | N/A | N/A | N/A |
| hIgG4 | | | | | | | | |
| Prot. A | 89 | 70 | 77 | 87 | 27 | 6 | 7 | 8 |
| DX249 | 61 | 42 | 43 | 30 | 10 | 4 | 53 | 59 |
| DX253 | 60 | 49 | 49 | 53 | 23 | 4 | 45 | 62 |
| DX596 | 65 | 44 | N/A | 61 | 17 | 2 | 55 | 67 |
| DX597 | 71 | 50 | N/A | 62 | 13 | 3 | 56 | 67 |
| DX1070 | 59 | N/A | 14 | N/A | N/A | N/A | N/A | N/A |
| DX1071 | 1 | N/A | 5 | N/A | N/A | N/A | N/A | N/A |
| DX1072 | 15 | N/A | 11 | N/A | N/A | N/A | N/A | N/A |

N/A = not tested

Species specificity results (Table 14) showed no significant binding of goat, bovine, and mouse IgG to DX249, DX253, DX597 and DX1070. DX596 showed a slightly elevated level of binding to these IgG targets of 5-8% above background. In comparison, Protein A showed binding to goat, bovine and mouse IgG.

TABLE 14
% IgG Capture - Batch Binding: Species Specificity

| | Human IgG | Goat IgG | Bovine IgG | Mouse IgG |
|---|---|---|---|---|
| Prot. A | 81 | 20 | 60 | 69 |
| DX249 | 74 | 0 | 0 | 2 |
| DX253 | 71 | 0 | 0 | 2 |
| DX596 | 72 | 8 | 5 | 5 |
| DX597 | 77 | 0 | 0 | 3 |
| DX1070 | 91 | 0 | 0 | 0 |
| DX1071 | 45 | 3 | 0 | 8 |
| DX1072 | 49 | 4 | 0 | 0 |

### EXAMPLE 12

### Static and Dynamic Capacity of DX249 and DX253 vs. Ligand Density

Polypeptides DX249 and DX253 were immobilized onto NHS-activated Sepharose 4 Fast Flow media (as previously described) at nominal ligand densities of 0.2, 1.0, 2.0 and 4.0 μmole ligand/ml media. These media were used to determine the effect of ligand density on human IgG binding capacity in both static (filterplate) and dynamic (mini-column) modes with commercial immobilized recombinant Protein A (Repligen, IPA400) at a single density as control. Actual ligand densities obtained ranged from 0.17 to 3.2 μmole/ml media as confirmed by quantitative amino acid analysis.

Static (Filterplate) Mode

Using a 96-well assay filterplate (Millipore MultiScreen-HV, with a 0.45 μm Durapore membrane, product No. MAHVN4510), 10 μl (packed bed volume) of each of the media to be tested, DX249 (at four densities), DX253 (at four densities), hydrolyzed NHS-Sepharose 4 FF, and rProtein A (RepliGen Corp. product No. IPA-400) were suspended in 100 μl of PBST (10 mM Phosphate buffer, 150 mM NaCl, 0.05% Tween-20, pH 7.5) containing varying amounts of human IgG (0.0, 1.25, 2.5 and/or up to 20.0 mg/ml). After incubation of the samples, with shaking, at room temperature for one hour, the supernatant liquid from each well, containing unbound IgG, was collected by centrifugation into the cognate well of a collection plate. The assay plate was washed three times with PBST (200 μl/wash) and bound IgG was eluted from the media samples by incubation for 30-60 minutes at room temperature in 100 μl Elution Buffer (100 mM CAPS, pH 11.5). Eluates were collected by centrifugation for analysis. Protein concentrations were determined from A₂₈₀ measurements and plotted as a function of mg human IgG bound per ml resin vs. mg human IgG applied per ml resin.

The amount of IgG captured (estimated from IgG recovered in the elution step) by the various media is dependent upon both the IgG concentration and the ligand density. In general, with both DX249 and DX253 apparent relative static capacities increased with ligand densities and target concentration. Under these static conditions, where both ligand and target concentrations are close to K_{D} values, equilibrium binding is established and total capacities cannot be accurately determined. However, over the IgG range of 0 to 25 mg IgG applied per ml resin (0 to 2.5 mg/ml target added to 10 μl of resin), capture of IgG is linear. Beyond this point, up to a level of 200 mg IgG applied per ml resin, saturation becomes more pronounced. Table 15 shows apparent relative static binding performance of both DX249 and DX253 media. At ligand densities around 1 μmole/ml DX249 and DX253 media is comparable to that of Protein A media at a target concentration of 25 mg IgG applied per ml resin (2.5 mg/ml target added to 10 μl of resin). At higher ligand densities (especially with higher target concentrations) the apparent relative static capacities of the DX-peptide media significantly exceed (by 2- to 3-fold) that of the Protein A media.

Dynamic (Mini-Column) Mode

Mini-chromatography columns (Omnifit, 6319) 3 mm×25 mm (internal diam.×length), 0.17 ml bed volume, were packed with various DX249, various DX253, and Protein A media for dynamic performance testing. Human IgG (ICN, 55908) was prepared in PBS pH 7.2 at 2.5 mg/ml. The IgG solution was loaded onto the column at a flow rate of 0.2 ml/minute (170 cm/hr). The absorbance of the column eluate was monitored at both 214 and 280 nm and fractions were collected across the entire run. At the end of the loading period, the column was washed with PBS until the absorbance at 280 nm of the column eluant returned to baseline. IgG bound to the column was eluted using 30 mM H₃PO₄, 150 mM NaCl, pH 2.0 followed by PBS wash to baseline followed by cleaning with 0.1M CAPS, pH 11.5. Neutralizing the pH of the eluted fractions was unnecessary since the total IgG eluted was determined by total protein absorbance at 280 nm.

To establish total media capacities, IgG was loaded onto the columns until the absorbances of the column load and flow through streams were identical (loading to full capacity). After washing the column to baseline, bound IgG was eluted and the total amount captured from this elution fraction determined. The total capacity of the media (mg IgG/ml media) as total IgG captured divided by the column bed volume was calculated.

Additionally, column capacities at 10% breakthrough were calculated from the online A₂₈₀ measurements of column flowthrough streams. The A₂₈₀ curves obtained for the column of interest and for an equivalent “blank” column containing hydrolyzed Sepharose FF 4 media were aligned. The difference in areas between these curves (for a retained and non-retained target) from the time of injection to the time at which the A₂₈₀ value for the column of interest had risen to 10% of the maximum A₂₈₀ absorbance (100%) obtained for the load solution at 100% IgG capture saturation (10% breakthrough) was graphically integrated. A standard curve was used to relate the integrated area to mg IgG bound and capacity calculated as total IgG bound divided by column bed volume.

Table 15 summarizes the results of these studies for the nine different affinity media tested: DX249 and DX253, each at four different ligand densities as measured by amino acid analysis, and recombinant Protein A (Repligen, #IPA400) at an undefined density. For both DX249 and DX253 media, both static and dynamic capacity values increase with increasing ligand densities. Dynamic capacities similar to that for the rprotein A media are achieved for ligand densities of about 2 to 3 μmole/ml. For each ligand density tested, static and total dynamic capacities, as measured by elution, are similar. For the two DX media at all ligand densities, capacities at 10% breakthrough are about 50% (±20%) of total dynamic capacities; as compared to about 75% for rProtein A. This small difference may result from kinetic or ligand availability difference between the media.

TABLE 15

| | Ligand | | | |
|---|---|---|---|---|
| | Density | Capacity (mg IgG/mL resin) | | |
| Ligand | (μM/mL) | Static | Total Dynamic | 10% Breakthrough |
| DX-253 | 0.17 | 1.1 | 0.6 | 0.1 |
| | 0.84 | 8.5 | 5.8 | 2.6 |
| | 1.45 | 13.5 | 6.4 | 2.9 |
| | 2.75 | 17.4 | 24.1 | 8.5 |
| DX-249 | 0.19 | 1.2 | 1.2 | 1.1 |
| | 0.86 | 9.5 | 8.4 | 4.4 |
| | 1.63 | 15.4 | 10.3 | 5.9 |
| | 3.20 | 19.0 | 16.4 | 5.5 |
| rProA | (not | 11.1 | 16.3 | 12.9 |
| | determined) | | | |

### EXAMPLE 13

### DX253 Column Cycling (Re-use)

DX253 peptide was immobilized onto NHS-activated 4 Fast Flow to a ligand density of 2.9 μmole/ml as determined by quantitative amino acid analysis. This affinity resin was packed into a 4.6 mm×6 cm (internal diam.×length, 1.0 ml bed volume) PEEK column (Isolation Technologies, Inc., 5050IP-08046-006-20). Prior to performing total capacity and cycling studies, the column/affinity matrix was pre-conditioned with 100 mg BSA load, 0.1M citrate, pH 3.0, 3M guanidine-HCl, 20 mM phosphate, pH 7.2 and re-equilibrated into PBS, pH 7.2.

Total Capacity

To establish total dynamic binding capacity, polyclonal human IgG (ICN, 55908) was prepared in PBS, pH 7.2 containing 0.05% NaN3 at 2.5 mg/ml. A total of 75 mg (30 ml) was loaded at 0.21 ml/minute (75 cm/hr) to capacity (until the absorbance at 280 nm of the load was equivalent to that of the column flowthrough). Fractions were collected across the entire run. Unbound IgG was washed to baseline at 0.42 ml/minute (150 cm/hour) with PBS, pH 7.2. Bound IgG was eluted with 0.1M citrate, pH 3.0 buffer and immediately neutralized with minimal addition of 1M HEPES, pH 9.0 buffer to ˜pH 7.0. Following an additional wash with PBS, pH 7.2, the column/affinity media was cleaned with 3M guanidine-HCl, 20 mM phosphate, pH 7.2 and re-equilibrated back into PBS, pH 7.2. All post-load wash, elution and cleaning steps were performed at a flow rate of 0.42 ml/minute (150 cm/hour). Total mg IgG bound was determined by measuring the absorbance at 280 nm of the elution fraction calculated using an extinction coefficicent of 1.4. Initial (T=0, prior to initiating column cycling) total dynamic capacity measured 19.8 mg human IgG per ml affinity resin. This determination was repeated 4 additional times during the course of 95 cycles to assess the relative performance of the affinity column after repeated use and re-use. Mass balance recovery for these 5 total capacity determinations averaged 98%. Table 16 shows the % relative column performance calculated, based on total capacity at T=0 of 19.8 mg/ml, or 100%. The column retained approximately 90% relative performance with respect to total dynamic capacity after 95 cycles. This procedure was used for cycles 0, 26, 66, 86, and 96, as shown in Table 16.

Column Cycling

Human IgG was spiked into fresh cell culture media (DMEM) containing 10% fetal bovine serum (ultra low) to 0.5 mg/ml. 2 ml (1.0 mg IgG) was loaded onto the column which was pre-equilibrated with PBS, pH 7.2. Loading was performed at 0.21 ml/minute (75 cm/hour) and all subsequent steps (wash, elution and cleaning) were performed at 0.42 ml/minute (150 cm/hour). Unbound sample was washed to baseline with PBS, pH 7.2, followed by elution with 0.1M citrate, pH 3.0, wash with PBS, pH 7.2, cleaning with 3M guanidine-HCl, 20 mM phosphate, pH 7.2 and re-equilibration back into PBS, pH 7.2. A total of 19 series of 5 cycles each (95 cycles total) were run. Table 16 shows % relative performance at every tenth cycle, with respect to elution peak area recovery as compared to the initial cycle (cycle 1 peak area=100%). This procedure was used for cycles 1-25, 27-65, 67-85, and 87-95.

At approximately 5% of total dynamic capacity load (1.0 mg IgG), the column performance remains unchanged despite the ˜10% decline in total capacity performance. Cycles 1, 26, 41, 66, 80 and 95 were collected and the eluted IgG fraction was immediately neutralized with a minimal addition of 1M Hepes, pH 9.0 buffer to pH ˜7.0. SDS-PAGE analysis (4-12% Tris-Glycine gel, denaturing/non-reducing, Coomassie stained) showed complete and efficient capture of the human IgG from cell culture media. The eluted fraction was equivalent in purity to the purified standard and remained consistent over the course of the study.

TABLE 16
Relative Performance of DX253 Sepharose Column
at Different Cycles

| Cycling | % Relative | Total Capacity | % Relative |
|---|---|---|---|
| Run No. | Performance | Cycle Run No. | Performance |
| 1 | 100 | 0 | 100 |
| 10 | 97.6 | 26 | 93.9 |
| 20 | 97.9 | 66 | 85.9 |
| 30 | 102.3 | 86 | 91.4 |
| 40 | 103.1 | 96 | 87.9 |
| 51 | 102.0 | | |
| 60 | 103.1 | | |
| 70 | 102.6 | | |
| 81 | 102.1 | | |
| 90 | 101.8 | | |

### EXAMPLE 14

### DX249 C-Terminal Truncation/PEO-linker Substitution

A series of six derivatives of DX249 (SEQ ID NO:144) was constructed to determine the possibility of truncating the nine C-terminal residues including the C-terminal spacer/linker (-PEGGGK, SEQ ID NO:168) and replacing it with a more hydrophilic, non-peptide polyethylene glycol-like linker, while maintaining chromatographic performance. Such derivatives may be more quickly and less expensively synthesized.

Table 17 shows the various DX249 derivative constructs. The terminal structures are defined below. DX905 was designed with a 31-atom polyethylene-glycol-like spacer/linker, however peptide synthesis of DX905 failed (primarily due to reduction of substitution as a result of crosslinking of the reactive functionalities on the resin).

TABLE 17
DX249 C-Terminal Truncation/PEO-Linker Constructs

| | | SEQ |
|---|---|---|
| | | ID |
| DX-No. | Amino Acid Sequence | NO: |
| DX249 | Ac-GDDHMCVYTTWGELIWCDNHEPGPEGGGK-NH₂ | 144 |
| DX877 | Ac-GDDHMCVYTTWGELIWCDNHEPGPEG-X-NH₂ | 162 |
| DX878 | Ac-GDDHMCVYTTWGELIWCDNHEPG-X-Su-X-NH₂ | 163 |
| DX905* | Ac-GDDHMCVYTTWGELIWCDNHEPG-X-Z-X-NH₂ | 164 |
| DX907 | Ac-GDDHMCVYTTWGELIWCDNH-X-NH₂ | 165 |
| DX909 | Ac-GDDHMCVYTTWGELIWCDNH-X-Su-X-NH₂ | 166 |
| DX911 | Ac-GDDHMCVYTTWGELIWCDNH-X-Z-X-NH₂ | 167 |

*Synthesis Failed

In the foregoing table, Ac-denotes N-terminal acetylation. The terminal groups are:

—X—NH₂═—NH(CH₂CH₂O)₂—CH₂CH₂—NH₂

—X—Su—X—NH₂═—NH—(CH₂CH₂O)₂—CH₂CH₂—NH—C:O—CH₂CH₂—C:O—NH(CH₂CH₂O)₂—CH₂CH₂—NH₂

—X—Z—X—NH₂ ═—NH—(CH₂CH₂O)₂—CH₂CH₂—NH—C:O—CH₂—O—(CH₂CH₂O)₂—C:O—NH—(CH₂CH₂O)₂—CH₂CH₂—NH₂

DX249, DX877, DX878, DX907, DX909 and DX911 were synthesized and immobilized onto NHS-activated sepharose 4 fast flow to an average ligand density of 1.9 μmole/ml determined by quantitative amino acid analysis. These were subsequently tested in batch filterplate format for relative static capacity, human IgG subtype and species specificity as previously described. In addition, total dynamic capacities were determined for comparison. Table 18 is a summary of the data. The static capacity (mg IgG bound per ml resin) was measured at the 2.5 mg/ml target concentration (25 mg human IgG applied per ml resin). Sequences DX907, DX909 and DX911 show approximately a 2-fold decrease in apparent relative static capacity compared to DX249, DX877 and DX878. These sequences are truncated to the final varied amino acid after the second cysteine and the truncation may result in decreased affinity. Percentage capture of polyclonal human IgG and human IgG subtypes show a similar trend. In general, sequences DX877 and DX878 show similar binding properties to DX249. Species specificity testing showed no significant binding of bovine, goat and mouse IgG to this series of derivatives (data not shown).

TABLE 18
DX249 C-Terminal Truncation/PEG-Like Linker Substitution
Batch Binding (% Capture of Total Load)

| | | | | | | | Dy- |
|---|---|---|---|---|---|---|---|
| | | | | | | Static | namic |
| | | | | | | Cap. | Cap. |
| | hIgG | hIgG1 | hIgG2 | hIgG3 | hIgG4 | mg/ml | mg/ml |
| DX249 | 64 | 87 | 77 | 5 | 50 | 24 | 13 |
| DX877 | 61 | N/D | N/D | N/D | N/D | 20 | 8 |
| DX878 | 63 | N/D | N/D | N/D | N/D | 23 | 9 |
| DX907 | 49 | 65 | 54 | 7 | 35 | 10 | 5 |
| DX909 | 49 | 66 | 54 | 9 | 33 | 11 | N/D |
| DX911 | 43 | 58 | 46 | 6 | 25 | 9 | N/D |

N/D = Not Determined

### EXAMPLE 15

### DX249 N-Terminal Truncation/Residue Substitution

In general, the synthesis of DX249, having the sequence Ac-GDDHMCVYTTWGELIWCDNHEPGPEGGGK-NH₂ (SEQ ID NO:144) is difficult using standard Fmoc methods. The synthesis and folding are problematic, primarily due to beta rearrangement at the Asp-Asp-His position at the N-terminus. Accordingly, a series of N-terminally truncated derivatives of DX249 was designed and synthesized. Table 19 shows the various DX249 constructs.

TABLE 19

DX249 N-Terminal Truncation/Residue Substitution
Constructs

| | | SEQ |
|---|---|---|
| | | ID |
| DX-No. | Amino Acid Sequence | NO: |
| DX249 | Ac-GDDHMCVYTTWGELIWCDNHEPGPEGGGK-NH₂ | 144 |
| DX1062 | Ac-DHMCVYTTWGELIWCDNHEPGPEGGGK-NH₂ | 169 |
| DX1063 | Ac-EHMCVYTTWGELIWCDNHEPGPEGGGK-NH₂ | 170 |
| DX1064 | Ac-ACVYTTWGELIWCDNHEPGPEGGGK-NH₂ | 171 |
| DX1065* | Ac-TCVYTTWGELIWCDNHEPGPEGGGK-NH₂ | 172 |
| DX1066* | Ac-ECVYTTWGELIWCDNHEPGPEGGGK-NH₂ | 173 |
| DX1067 | Ac-VCVYTTWGELIWCDNHEPGPEGGGK-NH₂ | 174 |
| DX1068 | Ac-[Nle]CVYTTWGELIWCDNHEPGPEGGGK-NH₂ | 175 |
| DX1069 | Ac-CVYTTWGELIWCDNHEPGPEGGGK-NH₂ | 176 |

*Not Immobilized/Tested [Nie]=norleucine

These DX249 sequence variants (including unmodified DX249) were synthesized and immobilized (with the exception of DX1065 and DX1066) onto NHS activated sepharose 4 fast flow media to an average ligand density of 1.9 μmole/ml as determined by quantitative amino acid analysis. As previously described, these were tested in batch filterplate format for relative binding efficiency for human IgG subtype and species specificity. Table 20 is a summary of the data. In general, all the DX249 variants show a similar binding profile to DX249. Binding efficiency initially tends to appear to decrease with more extensive truncation. However, sequence DX1069, which has all residues prior to the first cysteine truncated, shows binding properties almost identical to those of DX249. Species specificity testing showed no significant binding of bovine, goat and mouse IgG to series of derivatives (data not shown). These data and the data from the C-terminal truncation/linker substitution tests (Example 14) indicate that suitable synthetic Fc-binding ligands having only 12 to 18 total residues can be produced according to these examples. Such ligands may be more easily and efficiently synthesized using standard methods.

TABLE 20
DX249 N-Terminal Truncation/Residue Substitution
Batch Binding (% Capture of Total Load)

| | hIgG | hIgG1 | hIgG2 | hIgG3 | hIgG4 |
|---|---|---|---|---|---|
| DX249 | 82 | 84 | 80 | 3 | 67 |
| DX1062 | 73 | 73 | 63 | 0 | 49 |
| DX1063 | 71 | 73 | 63 | 0 | 45 |
| DX1064 | 73 | 70 | 63 | 0 | 39 |
| DX1067 | 44 | 33 | 28 | 0 | 25 |
| DX1068 | 36 | 33 | 25 | 0 | 21 |
| DX1069 | 74 | 77 | 68 | 2 | 51 |

### EXAMPLE 16

### DX253 N-Terminal Truncation/Residue Substitution

The synthesis of DX253, having the sequence Ac-GDRRACSRDWSGALVWCAGHEPGPEGGGK-NH₂ (SEQ ID NO:145), is also difficult, primarily due to the N-terminal Arg-Arg positions giving the possibility of deletion sequences. Therefore, a series of nine derivatives of DX253 was constructed (similar to the variants for DX249, described supra) to determine the possibility of truncation and/or various residue substitutions in the five amino-terminal residue positions of the sequence. The remaining sequence including the C-terminal peptide spacer/linker was retained. Table 21 shows the various DX253 constructs.

TABLE 21

DX253 N-Terminal Truncation/Residue Substitution
Constructs

| | | |
|---|---|---|
| | | SEQ |
| | | ID |
| DX-No. | Amino Acid Sequence | NO: |

| | | |
|---|---|---|
| DX-253 | Ac-GDRRACSRDWSGALVWCAGHEPGPEGGGK-NH₂ | 145 |
| DX1139 | Ac-SRACSRDWSGALVWCAGHEPGPEGGGK-NH₂ | 177 |
| DX1140 | Ac-RRACSRDWSGALVWCAGHEPGPEGGGK-NH₂ | 178 |
| DX1141 | Ac-ERACSRDWSGALVWCAGHEPGPEGGGK-NH₂ | 179 |
| DX1142 | Ac-ACSRDWSGALVWCAGHEPGPEGGGK-NH₂ | 180 |
| DX1143 | Ac-TCSRDWSGALVWCAGHEPGPEGGGK-NH₂ | 181 |
| DX1144 | Ac-ECSRDWSGALVWCAGHEPGPEGGGK-NH₂ | 182 |
| DX1145 | Ac-VCSRDWSGALVWCAGHEPGPEGGGK-NH₂ | 183 |
| DX1146 | Ac-GCSRDWSGALVWCAGHEPGPEGGGK-NH₂ | 184 |
| DX1147 | Ac-CSRDWSGALVWCAGHEPGPEGGGK-NH₂ | 185 |

These DX253 sequence variants (including unmodified DX253) were synthesized and immobilized onto NHS activated sepharose 4 fast flow media to a target ligand density of 2.0 μmole/ml. As previously described, these were tested in batch filterplate format for relative binding efficiency for human IgG subtype and species specificity. Table 22 is a summary of the data.

In general, all the DX253 variants show a similar binding profile to DX253. Binding efficiency initially tends to decrease with sequences DX1139 to DX1141. However, sequences DX1142 through DX1147 show binding efficiencies similar to DX253. Of particular interest is DX1147, which has all residues prior to the first cysteine truncated and shows binding properties almost identical to those of DX253. This DX253-derived peptide is more easily and efficiently synthesized than is DX253.

The varying degree of binding may be due to, in part, to lower coupling efficiencies as a result of varying degrees of solubility in aqueous coupling conditions. In particular, sequences DX1139, DX1140, DX1146 and DX1147 were not fully soluble in aqueous coupling buffer. However, coupling efficiencies of all nine DX253 variants monitored by rp-HPLC was high and averaged>85%. Actual ligand densities were not determined. Species specificity testing showed no significant binding of bovine, goat and mouse IgG to this series of derivatives (data not shown).

TABLE 22
DX253 N-Terminal Truncation/Residue Substitution
Batch Binding (% Capture of Total Load)

| | hIgG | hIgG1 | hIgG2 | hIgG3 | hIgG4 |
|---|---|---|---|---|---|
| DX253 | 79 | 88 | 80 | 0 | 58 |
| DX1139 | 36 | 43 | 34 | 0 | 22 |
| DX1140 | 40 | 43 | 29 | 0 | 20 |
| DX1141 | 21 | 17 | 9 | 0 | 2 |
| DX1142 | 57 | 66 | 55 | 0 | 39 |
| DX1143 | 81 | 88 | 83 | 0 | 55 |
| DX1144 | 81 | 88 | 84 | 0 | 61 |
| DX1145 | 66 | 74 | 63 | 0 | 42 |
| DX1146 | 73 | 83 | 80 | 0 | 50 |
| DX1147 | 74 | 83 | 76 | 0 | 47 |

Following the foregoing description, the characteristics important for affinity-binding molecules permitting detection or separation of Fc-region polypeptides or molecules including Fc-region polypeptides in or from any solution, can be appreciated. Additional binding-molecule embodiments of the invention and alternative methods adapted to a particular solution or feed stream will be evident from studying the foregoing description. All such embodiments and obvious alternatives are intended to be within the scope of this invention, as defined by the claims that follow.

Each of the publications referred to above is hereby incorporated by reference in its entirety.

1. An isolated polypeptide comprising an amino acid sequence of the formulae I, II, III or IV:
Z₁—X₁—X₂—X₃—X₄—W—C—Z₂(SEQ ID NO:4); I.
wherein,
Z₁ is a polypeptide of at least 6 amino acids;
X₁ is G, H, N, R, or S;
X₂ is A, D, E, F, I, M, or S;
X₃ is A, I, L, M, or V;
X₄ is I, M, T, or V;
Z₂ is a polypeptide of at least one amino acid or is absent; and
Z₁ contains at least one cysteine residue such that formation of a disulfide bond with the invariant cysteine residue forms a cyclic peptide of 12 amino acids;
Z₁—X—W—Z₂—W—Z₃(SEQ ID NO:5) II.
wherein,
Z₁ is a polypeptide of at least one amino acid or is absent;
X is F or Y;
Z₂ is a tripeptide; and
Z₃ is a polypeptide of at least one amino acid; and
wherein at least two of the polypeptides Z₁, Z₂, and Z₃ contain a cysteine residue, such that formation of a disulfide bond between such cysteine residues forms a cyclic peptide of 7-12 amino acids;
Z₁—W—Z₂—W—W—Z₃(SEQ ID NO:6); III.
wherein,
Z₁ is a polypeptide of at least one amino acid;
Z₂ is a tripeptide; and
Z₃ is a polypeptide of at least one amino acid;
wherein at least two of the polypeptides Z₁, Z₂, and Z₃ contain a cysteine residue, such that formation of a disulfide bond between such cysteine residues forms a cyclic peptide of 8-12 amino acids, with the proviso that where Z₁ contains a cysteine, then Z₂ does not contain a cysteine, and where Z₂ contains a cysteine, it is the middle residue of the tripeptide and Z₃ also contains a cysteine;
Z₁—P—Xₗ—W—X₂—C—X₃—X₄—X₅(SEQ ID NO:7); IV.
wherein,
Z₁ is a polypeptide of at least one amino acid and includes a cysteine residue;
X₁ is A, E, R, S, or T;
X₂ is F, W, or Y;
X₃ is D, E, L, M, or Q;
X₄ is H, W, or Y;
X₅ is F or Y; and
wherein the cysteine residue in Z₁ and the cysteine residue between X₂ and X₃ form a cyclic peptide of 10-12 amino acids;
wherein said polypeptide binds an immunoglobulin Fc region.

2. The polypeptide according to claim 1, wherein
(a) said polypeptide comprises an amino acid sequence of the formula:
Z₁—X₁—X₂—X₃—X₄—W—C—Z₂(SEQ ID NO:4); I.
wherein,
X₁ is G;
X₂ is A or E;
X₃ is L;
X₄ is I or V; or
(b) said polypeptide comprises an amino acid sequence of the formula:
Z₁—X—W—Z₂—W—Z₃(SEQ ID NO:5) II.
wherein,
X is F or Y; and
wherein Z₂ is a peptide of the formula: X₁—X₂—X₃, wherein,
X₁ is C or Y;
X₂ is C, K, N, or T, with the proviso that X₂ is not C if X₁ is C, and
X₃ is F, I, K, Q, or V.

3. The polypeptide according to claim 1 wherein said polypeptide comprises an amino acid sequence selected from the group consisting of:

| | |
|---|---|
| R-R-A-C-S-R-D-W-S-G-A-L-V-W-C-A-G-H | (SEQ ID NO:14) |
| W-G-E-C-T-V-T-S-Y-G-E-L-I-W-C-G-G-L | (SEQ ID NO:15) |
| S-S-A-C-A-F-D-P-M-G-A-V-I-W-C-T-Y-D | (SEQ ID NO:16) |
| L-L-E-C-A-Y-N-T-S-G-E-L-I-W-C-N-G-S | (SEQ ID NO:17) |
| P-D-D-C-S-I-H-F-S-G-E-L-I-W-C-E-P-L | (SEQ ID NO:18) |
| L-G-E-C-T-V-T-S-Y-G-E-L-I-W-C-G-G-L | (SEQ ID NO:19) |
| W-G-E-C-T-V-T-S-Y-G-E-L-I-W-C-G-G-H | (SEQ ID NO:20) |
| D-H-M-C-V-Y-T-T-W-G-E-L-I-W-C-D-D-H | (SEQ ID NO:21) |
| W-G-E-C-T-V-T-S-Y-G-E-L-I-W-C-G-G-L | (SEQ ID NO:22) |
| C-R-A-C-S-R-D-W-P-G-A-L-V-W-C-A-G-H | (SEQ ID NO:23) |
| R-R-A-C-S-R-D-W-S-G-A-L-V-W-G-A-G-H | (SEQ ID NO:24) |
| L-H-A-C-A-F-D-P-M-G-A-V-I-W-C-T-Y-D | (SEQ ID NO:25) |
| D-H-M-C-V-Y-T-T-W-G-E-L-M-W-C-D-N-H | (SEQ ID NO:26) |
| P-P-T-C-T-W-D-W-Q-G-I-L-V-W-C-S-G-H | (SEQ ID NO:27) |
| S-N-K-C-S-N-T-W-D-G-S-L-I-W-C-S-A-N | (SEQ ID NO:28) |
| F-P-E-C-T-F-D-M-E-G-F-L-I-W-C-S-S-F | (SEQ ID NO:29) |
| H-D-L-C-A-Q-A-P-F-G-D-A-T-W-C-D-L-R | (SEQ ID NO:30) |
| P-N-H-C-S-Y-N-L-K-S-E-L-I-W-C-Q-D-L | (SEQ ID NO:31) |
| P-L-D-C-A-R-D-I-H-N-S-L-I-W-C-S-L-G | (SEQ ID NO:32) |
| G-S-E-C-S-W-T-S-L-N-E-L-I-W-C-A-H-W | (SEQ ID NO:33) |
| W-P-D-C-S-F-T-V-Q-R-D-L-I-W-C-E-A-L | (SEQ ID NO:34) |
| S-H-S-C-A-Y-D-Y-A-H-M-L-V-W-C-T-H-F | (SEQ ID NO:35) |
| D-H-M-C-V-Y-T-T-W-G-E-L-I-W-C-D-N-H | (SEQ ID NO:36) |
| R-P-N-C-T-F-A-N-S-G-E-L-I-W-C-M-H-Y | (SEQ ID NO:37) |
| W-W-G-C-Q-F-D-W-R-G-E-L-V-W-C-P-Y-L | (SEQ ID NO:38) |
| G-G-V-C-S-Y-S-G-M-G-E-I-V-W-C-R-W-F | (SEQ ID NO:39) |
| A-L-M-C-S-H-D-M-W-G-S-L-I-W-C-K-H-F | (SEQ ID NO:40) |
| W-W-N-C-H-N-G-W-T-W-T-G-G-W-C-W-W-F | (SEQ ID NO:41) |
| Y-H-V-C-A-R-D-S-W-D-Q-L-I-W-C-E-A-F | (SEQ ID NO:42) |
| N-Y-W-C-N-F-W-Q-L-P-T-C-D-N-L | (SEQ ID NO:43) |
| Y-W-Y-C-K-W-F-S-E-S-A-S-C-S-S-R | (SEQ ID NO:44) |
| Y-W-Y-C-K-W-F-E-D-K-H-P-C-D-S-S | (SEQ ID NO:45) |
| Y-W-Y-C-S-W-F-P-D-R-P-D-C-P-L-Y | (SEQ ID NO:46) |
| N-Y-W-C-N-V-W-L-L-G-D-V-C-R-S-H | (SEQ ID NO:47) |
| L-Y-W-C-H-V-W-F-G-Q-H-A-W-Q-C-K-Y-P | (SEQ ID NO:48) |
| Y-W-K-C-K-W-M-P-W-M-C-G-F-D | (SEQ ID NO:49) |
| D-D-H-C-Y-W-F-R-E-W-F-N-S-E-C-P-H-G | (SEQ ID NO:50) |
| N-Y-W-C-N-I-W-G-L-H-G-C-N-S-H | (SEQ ID NO:51) |
| Y-W-F-C-Q-W-F-S-Q-N-H-T-G-F-R-D | (SEQ ID NO:52) |
| H-Y-W-C-D-I-W-F-G-A-P-A-C-Q-F-R | (SEQ ID NO:53) |
| S-G-D-C-G-F-W-P-R-I-W-G-L-C-M-D-N | (SEQ ID NO:54) |
| F-W-Y-C-K-W-F-Y-E-D-A-Q-C-S-H-D | (SEQ ID NO:55) |
| Y-Y-W-C-N-Y-W-G-L-C-P-D-Q | (SEQ ID NO:56) |
| S-Y-W-C-K-L-W-D-V-C-P-Q-S | (SEQ ID NO:57) |
| K-Y-W-C-N-L-W-G-V-C-P-A-N | (SEQ ID NO:58) |
| Q-Y-W-C-Y-Q-W-G-L-C-G-A-N | (SEQ ID NO:59) |
| K-Y-W-C-Q-Q-W-G-V-C-N-G-S | (SEQ ID NO:60) |
| K-Y-W-C-V-Q-W-G-V-C-P-E-S | (SEQ ID NO:61) |
| K-Y-W-C-M-Q-W-G-L-C-G-W-E | (SEQ ID NO:62) |
| H-F-W-C-E-V-W-G-L-C-P-S-I | (SEQ ID NO:63) |
| Q-Y-W-C-T-K-W-G-L-C-T-N-V | (SEQ ID NO:64) |
| A-Y-W-C-K-V-W-G-L-C-Q-G-E | (SEQ ID NO:65) |
| K-Y-W-C-N-L-W-G-V-C-P-A-N | (SEQ ID NO:66) |
| Q-Y-W-G-N-V-W-G-V-C-L-P-S | (SEQ ID NO:67) |
| H-Y-W-C-Q-Q-W-G-I-C-E-R-P | (SEQ ID NO:68) |
| R-Y-W-C-N-I-W-D-V-C-P-E-Q | (SEQ ID NO:69) |
| Q-Y-W-C-T-H-W-G-L-C-G-K-Y | (SEQ ID NO:70) |
| T-Y-W-C-T-K-W-G-L-C-P-H-N | (SEQ ID NO:71) |
| F-Y-W-C-G-Q-W-G-L-C-A-P-P | (SEQ ID NO:72) |
| G-Y-W-C-N-V-W-G-L-C-S-T-E | (SEQ ID NO:73) |
| R-Y-W-C-G-V-W-G-V-C-E-L-D | (SEQ ID NO:74) |
| K-F-W-C-T-I-W-G-V-C-H-M-P | (SEQ ID NO:75) |
| H-Y-W-C-Q-Q-W-G-I-C-E-R-P | (SEQ ID NO:76) |
| R-Y-W-C-N-I-W-D-V-C-P-E-Q | (SEQ ID NO:77) |
| F-Y-W-C-S-Q-W-G-L-C-K-Y-D | (SEQ ID NO:78) |
| H-Y-W-C-E-K-W-G-L-C-L-M-S | (SEQ ID NO:79) |
| H-Y-W-C-Q-K-W-G-V-C-P-T-D | (SEQ ID NO:80) |
| H-Y-W-C-S-L-W-G-V-C-D-I-N | (SEQ ID NO:81) |
| R-F-W-C-S-A-W-G-V-C-P-A | (SEQ ID NO:82) |
| S-Y-W-C-K-I-W-D-V-C-P-Q-S | (SEQ ID NO:83) |
| Q-Y-W-C-S-I-W-K-V-C-P-G-R | (SEQ ID NO:84) |
| Y-W-Y-C-E-W-F-G-A-C-L-N-D | (SEQ ID NO:85) |
| E-Y-W-C-K-Y-W-G-L-E-C-V-H-R | (SEQ ID NO:86) |
| K-Y-W-C-T-Q-W-G-L-K-C-D-K-Q | (SEQ ID NO:87) |
| K-Y-W-C-S-F-W-G-L-Q-C-K-T | (SEQ ID NO:88) |
| R-Y-W-C-N-F-W-G-V-N-C-D-A-N | (SEQ ID NO:89) |
| N-Y-W-C-T-H-W-G-V-M-G-L-D-H | (SEQ ID NO:90) |
| Y-W-F-C-K-W-F-P-S-Q-C-Q-F-M | (SEQ ID NO:91) |
| A-Y-W-C-K-Q-W-G-L-K-C-Q-L-G | (SEQ ID NO:92) |
| K-Y-W-C-K-F-W-G-L-E-C-K-V-G | (SEQ ID NO:93) |
| N-Y-W-C-T-E-W-G-L-N-C-N-N-K | (SEQ ID NO:94) |
| S-Y-W-C-E-K-W-G-L-T-C-E-T-H | (SEQ ID NO:95) |
| E-Y-W-C-R-I-W-G-L-Q-C-N-M-V | (SEQ ID NO:96) |
| K-Y-W-C-K-K-W-G-V-N-C-D-F-N | (SEQ ID NO:97) |
| K-Y-W-C-S-V-W-G-V-Q-C-P-H-S | (SEQ ID NO:98) |
| F-Y-W-C-T-K-W-G-L-E-C-I-H-S | (SEQ ID NO:99) |
| H-Y-W-C-Q-Q-W-G-L-M-C-F-E-T | (SEQ ID NO:100) |
| K-Y-W-C-K-R-W-G-L-M-C-N-G-G | (SEQ ID NO:101) |
| A-Y-W-C-M-T-W-G-V-P-C-I-S-W | (SEQ ID NO:102) |
| K-Y-W-C-K-K-W-G-V-N-C-D-F-N | (SEQ ID NO:103) |
| K-Y-W-C-S-V-W-G-V-Q-C-P-D-S | (SEQ ID NO:104) |
| K-Y-W-C-S-V-W-G-V-Q-C-P-H-S | (SEQ ID NO:105) |
| L-Y-W-C-T-K-W-G-V-T-C-Q-K-D | (SEQ ID NO:106) |
| T-Y-W-C-H-K-W-G-V-K-C-A-T-T | (SEQ ID NO:107) |
| T-Y-W-C-T-F-W-E-L-P-C-D-P-A | (SEQ ID NO:108) |
| K-Y-W-C-T-K-W-Q-L-N-C-E-E-V | (SEQ ID NO:109) |
| N-Y-W-C-H-F-W-Q-V-P-C-L-E-Q | (SEQ ID NO:110) |
| T-Y-W-C-V-V-W-N-V-P-C-S-T-D | (SEQ ID NO:111) |
| N-F-W-C-H-T-W-G-L-Q-C-N-D-L | (SEQ ID NO:112) |
| F-W-Y-C-Y-W-F-N-E-K-C-K-T-P | (SEQ ID NO:113) |
| G-F-W-C-T-F-W-G-V-T-C-E-A-G | (SEQ ID NO:114) |
| P-H-N-C-D-D-H-Y-W-Y-C-K-W-F | (SEQ ID NO:115) |
| E-M-T-C-S-S-H-Y-W-Y-C-T-W-M | (SEQ ID NO:116) |
| H-I-D-C-K-T-N-Y-W-W-C-R-W-T | (SEQ ID NO:117) |
| E-M-R-C-G-Q-H-F-W-Y-C-E-W-F | (SEQ ID NO:118) |
| N-Y-W-C-N-F-W-Q-L-P-T-C-D-N-L | (SEQ ID NO:119) |
| Y-W-Y-C-Q-W-F-Q-E-V-N-K-C-F-N-S | (SEQ ID NO:120) |
| Y-Y-W-C-R-H-W-F-P-D-F-D-C-V-H-S | (SEQ ID NO:121) |
| Y-W-Y-C-S-W-F-P-D-R-P-D-G-P-L-Y | (SEQ ID NO:122) |
| Y-W-Y-C-V-W-F-D-N-A-D-Q-C-V-H-H | (SEQ ID NO:123) |
| A-A-T-C-S-T-S-Y-W-Y-Y-Q-W-F-C-T-D-S | (SEQ ID NO:124) |
| Y-W-A-C-V-W-G-L-K-S-C-V-D-R | (SEQ ID NO:125) |
| Y-W-R-C-V-W-F-P-A-S-C-P-T | (SEQ ID NO:126) |
| D-W-Q-C-L-W-W-G-N-S-F-W-P-Y-C-A-N-L | (SEQ ID NO:127) |
| F-W-R-C-H-W-W-P-E-R-C-P-V-D | (SEQ ID NO:128) |
| N-P-M-C-W-K-K-S-W-W-E-D-A-Y-C-I-N-H | (SEQ ID NO:129) |
| S-W-V-C-W-K-A-K-W-W-E-D-K-R-C-A-P-F | (SEQ ID NO:130) |
| S-R-Q-C-W-K-E-L-W-W-T-D-Q-M-C-L-D-L | (SEQ ID NO:131) |
| S-F-R-C-Q-S-S-F-P-S-W-Y-C-D-Y-Y | (SEQ ID NO:132) |
| S-W-H-C-Q-N-T-Y-P-E-W-Y-C-Q-W-Y | (SEQ ID NO:133) |
| G-S-K-C-K-Q-T-G-F-P-R-W-W-C-E-H-Y | (SEQ ID NO:134) |
| D-G-V-C-G-P-R-G-F-G-P-A-W-F-C-M-H-Y | (SEQ ID NO:135) |
| Y-S-H-C-A-T-H-Y-P-T-W-Y-C-L-H-F | (SEQ ID NO:136) |
| N-P-M-C-W-R-A-S-W-W-E-D-A-Y-C-I-N-H | (SEQ ID NO:186) |
| N-P-M-C-W-R-A-H-W-W-E-D-A-Y-C-I-N-H | (SEQ ID NO:187) |
| E-H-M-C-V-Y-T-T-W-G-E-L-I-W-C-D-N-H | (SEQ ID NO:188) |
| A-C-V-Y-T-T-W-G-E-L-I-W-C-D-N-H | (SEQ ID NO:189) |
| T-C-V-Y-T-T-W-G-E-L-I-W-C-D-N-H | (SEQ ID NO:190) |
| E-C-V-Y-T-T-W-G-E-L-I-W-C-D-N-H | (SEQ ID NO:191) |
| V-C-V-Y-T-T-W-G-E-L-I-W-C-D-N-H | (SEQ ID NO:192) |
| S-R-A-C-S-R-D-W-S-G-A-L-V-W-C-A-G-H | (SEQ ID NO:194) |
| E-R-A-C-S-R-D-W-S-G-A-L-V-W-C-A-G-H | (SEQ ID NO:195) |
| A-C-S-R-D-W-S-G-A-L-V-W-C-A-G-H | (SEQ ID NO:196) |
| T-C-S-R-D-W-S-G-A-L-V-W-C-A-G-H | (SEQ ID NO:197) |
| E-C-S-R-D-W-S-G-A-L-V-W-C-A-G-H | (SEQ ID NO:198) |
| V-C-S-R-D-W-S-G-A-L-V-W-C-A-G-H | (SEQ ID NO:199) |
| and | |
| G-C-S-R-D-W-S-G-A-L-V-W-C-A-G-H. | (SEQ ID NO:200) |

4. The polypeptide according to claim 1, wherein said polypeptide comprises an amino acid sequence selected from the group consisting of:

| | |
|---|---|
| RRACSRDWSGALVWCAGH; | (SEQ ID NO:14) |
| DHMCVYTTWGELIWCDNH; | (SEQ ID NO:36) |
| KYWCSFWGLQCKT; | (SEQ ID NO:88) |
| PVDCKHHFWWCYWN; | (SEQ ID NO:141) |
| DDHCYWFREWENSECPHG; | (SEQ ID NO:50) |
| YYWCNYWGLCPDQ; | (SEQ ID NO:56) |
| PHNCDDHYWYCKWF; | (SEQ ID NO:115) |
| SYWCKIWDVCPQS; | (SEQ ID NO:57) |
| KYWCNLWGVCPAN; | (SEQ ID NO:58) |
| AATCSTSYWYYQWFCTDS; | (SEQ ID NO:124) |
| TYWCTFWELPCDPA; | (SEQ ID NO:108) |
| YWYGWFPDRPECPLY; | (SEQ ID NO:143) |
| SWVCWKAKWWEDKRCAPF; | (SEQ ID NO:130) |
| NPMCWKKSWWEDAYCINH; | (SEQ ID NO:129) |
| SWNCAFHHNEMVWCDDG; | (SEQ ID NO:142) |
| Ac-GDDHMCVYTTWGELIWCDNHEPGPEGGGK-NH_{2;} | (SEQ ID NO:144) |
| Ac-AGKYWCSFWGLQCKTGTPGPEGGGK-NH_{2;} | (SEQ ID NO:146) |
| Ac-AGPVDCKHHFWWCYWNGTPGPEGGGK-NH_{2;} | (SEQ ID NO:153) |
| Ac-GDDDHCYWFREWFNSECPHGEPGPEGGGK-NH_{2;} | (SEQ ID NO:154) |
| Ac-GDRRACSRDWSGALVWCAGHEPGPEGGGK-NH_{2;} | (SEQ ID NO:145) |
| Ac-AGYYWCNYWGLCPDQGTPGPEGGGK-NH_{2;} | (SEQ ID NO:155) |
| Ac-AGPHNCDDHYWYCKWFPGPEGGGK-NH_{2;} | (SEQ ID NO:150) |
| Ac-AGSYWCKIWDVCPQSPGPEGGGK-NH_{2;} | (SEQ ID NO:147) |
| Ac-AGKYWCNLWGVCPANPGPEGGGK-NH_{2;} | (SEQ ID NO:148) |
| Ac-AGAATCSTSYWYYQWFCTDSPGPEGGGK-NH_{2;} | (SEQ ID NO:151) |
| Ac-AGTYWCTFWELPCDPAPGPEGGGK-NH_{2;} | (SEQ ID NO:149) |
| Ac-AGYWYCWFPDRPECPLYPGPEGGGK-NH_{2;} | (SEQ ID NO:152) |
| Ac-GDSWVCWKAKWWEDKRCAPFGTPGPEGGGK-NH_{2;} | (SEQ ID NO:156) |
| Ac-GDNPMCWKKSWWEDAYCINHGTPGPEGGGK-NH_{2;} | (SEQ ID NO:157) |
| Ac-GDSWNCAFHHNEMVWCDDGGTPGPEGGGK-NH_{2;} | (SEQ ID NO:158) |
| Ac-GDWGECTVTSYGELIWCGGLEPGPEGGGK-NH_{2;} | (SEQ ID NO:159) |
| Ac-GDNPMCWRASWWEDAYCINHEPGPEGGGK-NH_{2;} | (SEQ ID NO:160) |
| Ac-GDNPMCWRAHWWEDAYCINHEPGPEGGGK-NH_{2;} | (SEQ ID NO:161) |
| Ac-GDDHMCVYTTWGELIWGDNHEPGPEG-X-NH_{2;} | (SEQ ID NO:162) |
| Ac-GDDHMCVYTTWGELIWCDNHEPG-X-Su-X-NH_{2;} | (SEQ ID NO:163) |
| Ac-GDDHMCVYTTWGELIWCDNHEPG-X-Z-X-NH_{2;} | (SEQ ID NO:164) |
| Ac-GDDHMCVYTTWGELIWCDNH-X-NH_{2;} | (SEQ ID NO:165) |
| Ac-GDDHMCVYTTWGELWCDNH-X-Su-X-NH_{2;} | (SEQ ID NO:166) |
| Ac-GDDHMCVYTTWGELIWCDNH-X-Z-X-NH_{2;} | (SEQ ID NO:167) |
| Ac-DHMCVYTTWGELIWCDNHEPGPEGGGK-NH_{2;} | (SEQ ID NO:169) |
| Ac-EHMCVYTTWGELIWCDNHEPGPEGGGK-NH_{2;} | (SEQ ID NO:170) |
| Ac-ACVYTTWGELIWCDNHEPGPEGGGK-NH_{2;} | (SEQ ID NO:171) |
| Ac-TCVYTTWGELIWCDNHEPGPEGGGK-NH_{2;} | (SEQ ID NO:172) |
| Ac-ECVYYTTWGELTWCDNHEPGPEGGGK-NH_{2;} | (SEQ ID NO:173) |
| Ac-VCVYTTWGELIWCDNHEPGPEGGGK-NH_{2;} | (SEQ ID NO:174) |
| Ac-[Nle]CVYTTWGELIWCDNHEPGPEGGGK-NH_{2;} | (SEQ ID NO:175) |
| Ac-CVYTTWGELIWCDNHEPGPEGGGK-NH_{2;} | (SEQ ID NO:176) |
| Ac-SRACSRDWSGALVWCAGHEPGPEGGGK-NH_{2;} | (SEQ ID NO:177) |
| Ac-RRACSRDWSGALVWCAGHEPGPEGGGK-NH_{2;} | (SEQ ID NO:178) |
| Ac-ERACSRDWSGALVWCAGHEPGPEGGGK-NH_{2;} | (SEQ ID NO:179) |
| Ac-ACSRDWSGALVWCAGHEPGPEGGGK-NH_{2;} | (SEQ ID NO:180) |
| Ac-TCSRDWSGALVWGAGHEPGPEGGGK-NH_{2;} | (SEQ ID NO:181) |
| Ac-ECSRDWSGALVWCAGHEPGPEGGGK-NH_{2;} | (SEQ ID NO:182) |
| Ac-VCSRDWSGALVWCAGHEPGPEGGGK-NH_{2;} | (SEQ ID NO:183) |
| Ac-GCSRDWSGALVWCAGHEPGPEGGGK-NH_{2;} | (SEQ ID NO:184) |
| and | |
| Ac-CSRDWSGALVWCAGHEPGPEGGGK-NH_{2,} | (SEQ ID NO:185) |

wherein: Ac- denotes N-terminal acetylation; —NH₂ denotes C-terminal amidation; [Nle] denotes norleucine; —X—NH₂ dentoes the C-terminal group —NH—(CH₂CH₂O)₂—CH₂CH₂—NH₂; —X—Su—X—NH₂ denotes the C-terminal group —NH—(CH₂CH₂O)₂—CH₂CH₂—NH—C:O—CH₂CH₂—C:O—NH—(CH₂CH₂O)₂—CH₂CH₂—NH₂; and —X—Z—X—NH₂ denotes the C-terminal group —NH—(CH₂CH₂O)₂—CH₂CH₂—NH—C:O—CH₂—O—(CH₂CH₂O)₂—CH₂—C:O—NH—(CH₂CH₂O)₂—CH₂CH₂—NH₂.

5. An isolated polypeptide comprising an amino acid sequence selected from the group consisting of:

| | |
|---|---|
| F-C-N-C-W-G-S-H-E-F-T-F-C-V-D-D | (SEQ ID NO:137) |
| P-G-W-C-Y-S-D-I-W-G-F-K-H-F-C-N-L-D | (SEQ ID NO:138) |
| D-S-S-C-I-K-H-H-N-K-V-T-C-F-F-P | (SEQ ID NO:139) |
| R-W-S-C-W-G-V-W-G-C-V-W-V | (SEQ ID NO:140) |
| P-V-D-C-K-H-H-F-W-W-C-Y-W-N | (SEQ ID NO:141) |
| S-W-N-C-A-F-H-H-N-E-M-V-W-C-D-D-G | (SEQ ID NO:142) |
| Y-W-Y-C-W-F-P-D-R-P-E-C-P-L-Y, | (SEQ ID NO:143) |

wherein said polypeptide binds an immunoglobulin Fc region.

6. A method for detecting a polypeptide target that comprises at least one immunoglobulin Fc region amino acid sequence in a solution suspected of containing it, said method comprising:
(a) contacting said solution with a polypeptide according to claim 1 or claim 5; and
(b) determining whether binding has occurred between said polypeptide and said polypeptide target.

7. A method for isolating a polypeptide target that comprises at least one immunoglobulin Fc region amino acid sequence in a solution containing said polypeptide target, said method comprising:
(a) preparing a binding polypeptide according to claim 1 or claim 5 by immobilizing the binding polypeptide on a solid support or conjugating the binding polypeptide with an affinity tag;
(b) contacting the solution containing said polypeptide target with the binding polypeptide of step (a); and
(c) separating the non-binding components of said solution from the binding polypeptide of step (b).

8. A method for removing a polypeptide target that comprises at least one immunoglobulin Fc region amino acid sequence from a solution containing said polypeptide target, said method comprising:
(a) preparing a binding polypeptide according to claim 1 or claim 5 by immobilizing the binding polypeptide on a solid support or conjugating the binding polypeptide with an affinity tag;
(b) contacting the solution containing said polypeptide target with the binding polypeptide prepared according to step (a); and
(c) separating the non-binding components of said solution from the binding polypeptide of step (b).

9. A solid-phase detection assay method for a polypeptide target containing an Fc-region polypeptide, comprising the steps:
(a) contacting a solution suspected of containing a polypeptide target comprising an Fc-region polypeptide with a binding molecule for target polypeptide target, which binding molecule is immobilized on a solid support,
(b) separating the target bound to the solid support from the unbound components of the solution,
(c) contacting the solid support with an Fc binding polypeptide according to claim 1 or claim 5 which is detectably labeled, and
(d) detecting binding of the labeled Fc binding polypeptide on said solid support.

10. The method according to any one of claims 6, 7, 8 or 9, wherein said solution is selected from the group consisting of whole blood, plasma, transgenic milk, eggs of transgenic birds, and conditioned media.

11. The method of claim 10, wherein the solution is transgenic milk obtained from a transgenic mouse, rat, goat, rabbit, sheep, or cow.

12. The method of claim 10, wherein the solution is egg from a transgenic chicken, quail, turkey, ostrich, or goose.

13. The method according to any one of claims 6, 7, 8 or 9, wherein said polypeptide target can be an antibody or an antibody fragment containing all or part of an Fc region.

14. The method according to claim 13, wherein said polypeptide target is an antibody.

15. The method according to claim 14, wherein said antibody is an immunoglobulin is selected from the group consisting of human IgG1, IgG2, IgG3, and IgG4.

16. The method according to any one of claims 7, 8 or 9, wherein said solid support is selected from the group consisting of cellulose, controlled-pore glass, silica, polystyrene, styrene divinyl benzene, agarose, and crosslinked agarose.

17. A recombinant bacteriophage expressing an exogenous polypeptide capable of binding to an immunoglobulin Fc region, said exogenous polypeptide comprising an amino acid sequence of any of the formulae I, II, III or IV:
Z₁—X₁—X₂—X₃—X₄—W—C—Z₂(SEQ ID NO:4); I.
wherein,
Z₁ is a polypeptide of at least 6 amino acids;
X₁ is G, H, N, R, or S;
X₂ is A, D, E, F, I, M, or S;
X₃ is A, I, L, M, or V;
X₄is I, M, T, or V;
Z₂ is a polypeptide of at least one amino acid or is absent; and
Z₁ contains at least one cysteine residue such that formation of a disulfide bond with the invariant cysteine residue forms a cyclic peptide of 12 amino acids;
Z₁—X—W—Z₂—W—Z₃(SEQ ID NO:5) II.
wherein,
Z₁ is a polypeptide of at least one amino acid or is absent;
X is F or Y;
Z₂ is a tripeptide; and
Z₃ is a polypeptide of at least one amino acid; and
wherein at least two of the polypeptides Z₁, Z₂, and Z₃ contain a cysteine residue, such that formation of a disulfide bond between such cysteine residues forms a cyclic peptide of 7-12 amino acids;
Z₁—W—Z₂—W—W—Z₃(SEQ ID NO:6); III.
wherein,
Z₁ is a polypeptide of at least one amino acid;
Z₂ is a tripeptide; and
Z₃ is a polypeptide of at least one amino acid;
wherein at least two of the polypeptides Z₁, Z₂, and Z₃ contain a cysteine residue, such that formation of a disulfide bond between such cysteine residues forms a cyclic peptide of 8-12 amino acids, with the proviso that where Z₁ contains a cysteine, then Z₂ does not contain a cysteine, and where Z₂ contains a cysteine, it is the middle residue of the tripeptide and Z₃ also contains a cysteine;
Z₁—P—X₁—W—X₂—C—X₃—X₄—X₅(SEQ ID NO:7); IV.
wherein,
Z₁ is a polypeptide of at least one arnino acid and includes a cysteine residue;
X₁ is A, E, R, S, or T;
X₂ is F, W, or Y;
X₃ is D, E, L, M, or Q;
X₄ is H, W, or Y;
X₅ is F or Y; and
wherein the cysteine residue in Z₁ and the cysteine residue between X₂ and X₃ form a cyclic peptide of 10-12 amino acids.

18. The recombinant bacteriophage according to claim 17, wherein said exogenous polypeptide comprises an amino acid sequence of the formula:
Z₁—X₁—X₂—X₃—X₄—W—C—Z₂(SEQ ID NO:4);
wherein,
X₁ is G;
X₂ is A or E;
X₃ is L;
X₄ is I or V; or
said exogenous polypeptide comprises an amino acid sequence of the formula:
Z₁—X—W—Z₂—W—Z₃(SEQ ID NO:5)
wherein,
X is F or Y; and wherein Z₂ is a peptide of the formula: X₁—X₂—X₃, wherein,
X₁ is C or Y;
X₂ is C, K, N, or T, with the proviso that X₂ is not C if X₁ is C, and
X₃ is F, I, K, Q, or V.

19. The recombinant bacteriophage according to claim 17, wherein said exogenous polypeptide comprises an amino acid sequence selected from the group consisting of:

| | |
|---|---|
| R-R-A-C-S-R-D-W-S-G-A-L-V-W-C-A-G-H | (SEQ ID NO:14) |
| W-G-E-C-T-V-T-S-Y-G-E-L-I-W-C-G-G-L | (SEQ ID NO:15) |
| S-S-A-C-A-F-D-P-M-G-A-V-I-W-C-T-Y-D | (SEQ ID NO:16) |
| L-L-E-C-A-Y-N-T-S-G-E-L-I-W-C-N-G-S | (SEQ ID NO:17) |
| P-D-D-C-S-I-H-F-S-G-E-L-I-W-C-E-P-L | (SEQ ID NO:18) |
| L-G-E-C-T-V-T-S-Y-G-E-L-I-W-C-G-G-L | (SEQ ID NO:19) |
| W-G-E-C-T-V-T-S-Y-G-E-L-I-W-C-G-G-H | (SEQ ID NO:20) |
| D-H-M-C-V-Y-T-T-W-G-E-L-I-W-C-D-D-H | (SEQ ID NO:21) |
| W-G-E-C-T-V-T-S-Y-G-E-L-I-W-C-G-G-L | (SEQ ID NO:22) |
| C-R-A-C-S-R-D-W-P-G-A-L-V-W-C-A-G-H | (SEQ ID NO:23) |
| R-R-A-C-S-R-D-W-S-G-A-L-V-W-C-A-G-H | (SEQ ID NO:24) |
| L-H-A-C-A-F-D-P-M-G-A-V-I-W-C-T-Y-D | (SEQ ID NO:25) |
| D-H-M-C-V-Y-T-T-W-G-E-L-M-W-C-D-N-H | (SEQ ID NO:26) |
| P-P-T-C-T-W-D-W-Q-G-I-L-V-W-C-S-G-H | (SEQ ID NO:27) |
| S-N-K-C-S-N-T-W-D-G-S-L-I-W-C-S-A-N | (SEQ ID NO:28) |
| F-P-E-C-T-F-D-M-E-G-F-L-I-W-C-S-S-F | (SEQ ID NO:29) |
| H-D-L-C-A-Q-A-P-F-G-D-A-T-W-G-D-L-R | (SEQ ID NO:30) |
| P-N-H-C-S-Y-N-L-K-S-E-L-I-W-C-Q-D-L | (SEQ ID NO:31) |
| P-L-D-C-A-R-D-I-H-N-S-L-I-W-C-S-L-G | (SEQ ID NO:32) |
| G-S-E-C-S-W-T-S-L-N-E-L-I-W-C-A-H-W | (SEQ ID NO:33) |
| W-P-D-C-S-F-T-V-Q-R-D-L-I-W-C-E-A-L | (SEQ ID NO:34) |
| S-H-S-C-A-Y-D-Y-A-H-M-L-V-W-C-T-H-F | (SEQ ID NO:35) |
| D-H-M-C-V-Y-T-T-W-G-E-L-I-W-C-D-N-H | (SEQ ID NO:36) |
| R-P-N-C-T-F-A-A-S-G-E-L-I-W-C-M-H-Y | (SEQ ID NO:37) |
| W-W-G-C-Q-F-D-W-R-G-E-L-V-W-C-P-Y-L | (SEQ ID NO:38) |
| G-G-V-C-S-Y-S-G-M-G-E-I-V-W-C-R-W-F | (SEQ ID NO:39) |
| A-L-M-C-S-H-D-M-W-G-S-L-I-W-C-K-H-F | (SEQ ID NO:40) |
| W-W-N-C-H-N-G-W-T-W-T-G-G-W-C-W-W-F | (SEQ ID NO:41) |
| Y-H-V-C-A-R-D-S-W-D-Q-L-I-W-C-E-A-F | (SEQ ID NO:42) |
| N-Y-W-C-N-F-W-Q-L-P-T-C-D-N-L | (SEQ ID NO:43) |
| Y-W-Y-C-K-W-F-S-E-S-A-S-C-S-S-R | (SEQ ID NO:44) |
| Y-W-Y-C-K-W-F-E-D-K-H-P-C-D-S-S | (SEQ ID NO:45) |
| Y-W-Y-C-S-W-F-P-D-R-P-D-C-P-L-Y | (SEQ ID NO:46) |
| N-Y-W-C-N-V-W-L-L-G-D-V-C-R-S-H | (SEQ ID NO:47) |
| L-Y-W-C-H-V-W-F-G-Q-H-A-W-Q-C-K-Y-P | (SEQ ID NO:48) |
| Y-W-K-C-K-W-M-P-W-M-C-G-F-D | (SEQ ID NO:49) |
| D-D-H-C-Y-W-F-R-E-W-F-N-S-E-C-P-H-G | (SEQ ID NO:50) |
| N-Y-W-C-N-I-W-G-L-H-G-C-N-S-H | (SEQ ID NO:51) |
| Y-W-F-C-Q-W-F-S-Q-N-H-T-C-F-R-D | (SEQ ID NO:52) |
| H-Y-W-C-D-I-W-F-G-A-P-A-C-Q-F-R | (SEQ ID NO:53) |
| S-G-D-C-G-F-W-P-R-I-W-G-L-C-M-D-N | (SEQ ID NO:54) |
| F-W-Y-C-K-W-F-Y-E-D-A-Q-C-S-H-D | (SEQ ID NO:55) |
| Y-Y-W-C-N-Y-W-G-L-C-P-D-Q | (SEQ ID NO:56) |
| S-Y-W-C-K-I-W-D-V-C-P-Q-S | (SEQ ID NO:57) |
| K-Y-W-C-N-L-W-G-W-C-P-A-N | (SEQ ID NO:58) |
| Q-Y-W-C-Y-Q-W-G-L-G-G-A-N | (SEQ ID NO:59) |
| K-Y-W-C-Q-Q-W-G-V-C-N-G-S | (SEQ ID NO:60) |
| K-Y-W-C-V-Q-W-G-V-C-P-E-S | (SEQ ID NO:61) |
| K-Y-W-C-M-Q-W-G-L-C-G-W-E | (SEQ ID NO:62) |
| H-F-W-C-E-V-W-G-L-C-P-S-I | (SEQ ID NO:63) |
| Q-Y-W-C-T-K-W-G-L-C-T-N-V | (SEQ ID NO:64) |
| A-Y-W-C-K-V-W-G-L-C-Q-G-E | (SEQ ID NO:65) |
| K-Y-W-C-N-L-W-G-V-C-P-A-N | (SEQ ID NO:66) |
| Q-Y-W-C-N-V-W-G-V-C-L-P-S | (SEQ ID NO:67) |
| H-Y-W-C-Q-Q-W-G-I-C-E-R-P | (SEQ ID NO:68) |
| R-Y-W-C-N-I-W-D-V-C-P-E-Q | (SEQ ID NO:69) |
| Q-Y-W-C-T-H-W-G-L-C-G-K-Y | (SEQ ID NO:70) |
| T-Y-W-C-T-K-W-G-L-C-P-H-N | (SEQ ID NO:71) |
| F-Y-W-C-G-Q-W-G-L-C-A-P-P | (SEQ ID NO:72) |
| G-Y-W-C-N-V-W-G-L-C-S-T-E | (SEQ ID NO:73) |
| R-Y-W-C-G-V-W-G-V-C-E-I-D | (SEQ ID NO:74) |
| K-F-W-G-T-I-W-G-V-C-H-M-P | (SEQ ID NO:75) |
| H-Y-W-C-Q-Q-W-G-I-C-E-R-P | (SEQ ID NO:76) |
| R-Y-W-C-N-I-W-D-V-C-P-E-Q | (SEQ ID NO:77) |
| F-Y-W-C-S-Q-W-G-L-C-K-Y-D | (SEQ ID NO:78) |
| H-Y-W-C-E-K-W-G-L-C-L-M-S | (SEQ ID NO:79) |
| H-Y-W-C-Q-K-W-G-V-C-P-T-D | (SEQ ID NO:80) |
| H-Y-W-C-S-L-W-G-V-C-D-I-N | (SEQ ID NO:81) |
| R-F-W-C-S-A-W-G-V-C-P-A | (SEQ ID NO:82) |
| S-Y-W-C-K-I-W-D-V-C-P-Q-S | (SEQ ID NO:83) |
| Q-Y-W-C-S-I-W-K-V-C-P-G-R | (SEQ ID NO:84) |
| Y-W-Y-C-E-W-F-G-A-C-I-N-D | (SEQ ID NO:85) |
| E-Y-W-C-K-Y-W-G-L-E-C-V-H-R | (SEQ ID NO:86) |
| K-Y-W-C-T-Q-W-G-L-K-C-D-K-Q | (SEQ ID NO:87) |
| K-Y-W-C-S-F-W-G-L-Q-C-K-T | (SEQ ID NO:88) |
| R-Y-W-C-N-F-W-G-V-N-C-D-A-N | (SEQ ID NO:89) |
| N-Y-W-C-T-H-W-G-V-M-C-L-D-H | (SEQ ID NO:90) |
| Y-W-F-C-K-W-F-P-S-Q-C-Q-F-M | (SEQ ID NO:91) |
| A-Y-W-C-K-Q-W-G-L-K-C-Q-L-G | (SEQ ID NO:92) |
| K-Y-W-C-K-F-W-G-L-E-C-K-V-G | (SEQ ID NO:93) |
| N-Y-W-C-T-E-W-G-L-N-C-N-N-K | (SEQ ID NO:94) |
| S-Y-W-C-E-K-W-G-L-T-C-E-T-H | (SEQ ID NO:95) |
| E-Y-W-C-R-I-W-G-L-Q-C-N-M-V | (SEQ ID NO:96) |
| K-Y-W-C-K-K-W-G-V-N-C-D-F-N | (SEQ ID NO:97) |
| K-Y-W-C-S-V-W-G-V-Q-C-P-H-S | (SEQ ID NO:98) |
| F-Y-W-C-T-K-W-G-L-E-C-I-H-S | (SEQ ID NO:99) |
| H-Y-W-C-Q-Q-W-G-L-M-C-F-E-T | (SEQ ID NO:100) |
| K-Y-W-C-K-R-W-G-L-M-C-N-G-G | (SEQ ID NO:101) |
| A-Y-W-C-M-T-W-G-V-P-C-I-S-W | (SEQ ID NO:102) |
| K-Y-W-C-K-K-W-G-V-N-C-D-F-N | (SEQ ID NO:103) |
| K-Y-W-C-S-V-W-G-V-Q-C-P-D-S | (SEQ ID NO:104) |
| K-Y-W-C-S-V-W-G-V-Q-C-P-H-S | (SEQ ID NO:105) |
| L-Y-W-C-T-K-W-G-V-T-C-Q-K-D | (SEQ ID NO:106) |
| T-Y-W-C-H-K-W-G-V-K-C-A-T-T | (SEQ ID NO:107) |
| T-Y-W-C-T-F-W-E-L-P-C-D-P-A | (SEQ ID NO:108) |
| K-Y-W-C-T-K-W-Q-L-N-C-E-E-V | (SEQ ID NO:109) |
| N-Y-W-C-H-F-W-Q-V-P-C-L-E-Q | (SEQ ID NO:110) |
| T-Y-W-C-V-V-W-N-V-P-C-S-T-D | (SEQ ID NO:111) |
| N-F-W-C-H-T-W-G-L-Q-C-N-D-L | (SEQ ID NO:112) |
| F-W-Y-C-Y-W-F-N-E-K-C-K-T-P | (SEQ ID NO:113) |
| G-F-W-C-T-F-W-G-V-T-C-E-A-G | (SEQ ID NO:114) |
| P-H-N-C-D-D-H-Y-W-Y-C-K-W-F | (SEQ ID NO:115) |
| E-M-T-C-S-S-H-Y-W-Y-C-T-W-M | (SEQ ID NO:116) |
| H-I-D-C-K-T-N-Y-W-W-C-R-W-T | (SEQ ID NO:117) |
| E-M-R-C-G-Q-H-F-W-Y-C-E-W-F | (SEQ ID NO:118) |
| N-Y-W-C-N-F-W-Q-L-P-T-C-D-N-L | (SEQ ID NO:119) |
| Y-W-Y-C-Q-W-F-Q-E-V-N-K-C-F-N-S | (SEQ ID NO:120) |
| Y-Y-W-C-R-H-W-F-P-D-F-D-C-V-H-S | (SEQ ID NO:121) |
| Y-W-Y-C-S-W-F-P-D-R-P-D-C-P-L-Y | (SEQ ID NO:122) |
| Y-W-Y-C-V-W-F-D-N-A-D-Q-C-V-H-H | (SEQ ID NO:123) |
| A-A-T-C-S-T-S-Y-W-Y-Y-Q-W-F-C-T-D-S | (SEQ ID NO:124) |
| Y-W-A-C-V-W-G-L-K-S-C-V-D-R | (SEQ ID NO:125) |
| Y-W-R-C-V-W-F-P-A-S-C-P-T | (SEQ ID NO:126) |
| D-W-Q-C-L-W-W-G-N-S-F-W-P-Y-C-A-N-L | (SEQ ID NO:127) |
| F-W-R-C-H-W-W-P-E-R-C-P-V-D | (SEQ ID NO:128) |
| N-P-M-C-W-K-K-S-W-W-E-D-A-Y-C-I-N-H | (SEQ ID NO:129) |
| S-W-V-C-W-K-A-K-W-W-E-D-K-R-C-A-P-F | (SEQ ID NO:130) |
| S-R-Q-C-W-K-E-L-W-W-T-D-Q-M-C-L-D-L | (SEQ ID NO:131) |
| S-F-R-C-Q-S-S-F-P-S-W-Y-C-D-Y-Y | (SEQ ID NO:132) |
| S-W-H-C-Q-N-T-Y-P-E-W-Y-C-Q-W-Y | (SEQ ID NO:133) |
| G-S-K-C-K-Q-T-G-F-P-R-W-W-C-E-H-Y | (SEQ ID NO:134) |
| D-G-V-C-G-P-R-G-F-G-P-A-W-F-C-M-H-Y | (SEQ ID NO:135) |
| Y-S-H-C-A-T-H-Y-P-T-W-Y-C-L-H-F | (SEQ ID NO:136) |
| N-P-M-C-W-R-A-S-W-W-E-D-A-Y-C-I-N-H | (SEQ ID NO:186) |
| N-P-M-C-W-R-A-H-W-W-E-D-A-Y-C-I-N-H | (SEQ ID NO:187) |
| E-H-M-C-V-Y-T-T-W-G-E-L-I-W-C-D-N-H | (SEQ ID NO:188) |
| A-C-V-Y-T-T-W-G-E-L-I-W-C-D-N-H | (SEQ ID NO:189) |
| T-C-V-Y-T-T-W-G-E-L-I-W-C-D-N-H | (SEQ ID NO:190) |
| E-C-V-Y-T-T-W-G-E-L-I-W-C-D-N-H | (SEQ ID NO:191) |
| V-C-V-Y-T-T-W-G-E-L-I-W-G-D-N-H | (SEQ ID NO:192) |
| S-R-A-C-S-R-D-W-S-G-A-L-V-W-C-A-G-H | (SEQ ID NO:194) |
| E-R-A-C-S-R-D-W-S-G-A-L-V-W-C-A-G-H | (SEQ ID NO:195) |
| A-C-S-R-D-W-S-G-A-L-V-W-C-A-G-H | (SEQ ID NO:196) |
| T-C-S-R-D-W-S-G-A-L-V-W-C-A-G-H | (SEQ ID NO:197) |
| E-C-S-R-D-W-S-G-A-L-V-W-C-A-G-H | (SEQ ID NO:198) |
| V-C-S-R-D-W-S-G-A-L-V-W-C-A-G-H | (SEQ ID NO:199) |
| and | |
| G-C-S-R-D-W-S-G-A-L-V-W-C-A-G-H. | (SEQ ID NO:200) |

20. A recombinant bacteriophage expressing an exogenous polypeptide capable of binding to an immunoglobulin Fc region, said exogenous polypeptide comprising an amino acid sequence selected from the group consisting of:

| | |
|---|---|
| F-C-N-C-W-G-S-H-E-F-T-F-C-V-D-D | (SEQ ID NO:137) |
| P-G-W-C-Y-S-D-I-W-G-F-K-H-F-C-N-L-D | (SEQ ID NO:138) |
| D-S-S-C-I-K-H-H-N-K-V-T-C-F-F-P | (SEQ ID NO:139) |
| R-W-S-C-W-G-V-W-G-C-V-W-V | (SEQ ID NO:140) |
| P-V-D-C-K-H-H-F-W-W-C-Y-W-N | (SEQ ID NO:141) |
| S-W-N-C-A-F-H-H-N-E-M-V-W-C-D-D-G | (SEQ ID NO:142) |
| Y-W-Y-C-W-F-P-D-R-P-E-C-P-L-Y. | (SEQ ID NO:143) |

21. A method for detecting a polypeptide target comprising at least one immunoglobulin Fc region amino acid sequence in a solution, comprising:
(a) contacting said solution with a bacteriophage according to claim 17 or claim 20; and
(b) determining whether binding has occurred between said polypeptide target and said bacteriophage.

22. The method according to claim 21, wherein said solution is selected from the group consisting of whole blood, plasma, transgenic milk, eggs of transgenic birds, and conditioned media.

23. The method according to claim 19, wherein said polypeptide target is an antibody or antibody fragment containing all or part of an Fc region.

24. The method according to claim 23, wherein said polypeptide target is an antibody.

25. The method according to claim 24, wherein said antibody is selected from the group consisting of human IgG1, IgG2, IgG3, and IgG4.

26. The method according to claim 12, wherein said bacteriophage is a phagemid.

27. Separation media comprising a chromatographic matrix material, and, immobilized thereon, a polypeptide according to any one of claims 1, 2, 3, 4 or 5.

28. The separation media of claim 27, wherein said chromatographic matrix material is selected from the group consisting of cellulose, silica gel-type resins or membranes, crosslinked polysaccharides, and agarose.

29. The separation media of claim 28, wherein said chromatographic matrix material is an amine-reactive chromatographic matrix material.

30. The separation media of claim 28, wherein said chromatographic matrix material is an aldehyde-functional methacrylate resin.

31. The separation media of claim 28, wherein said chromatographic matrix material is a formyl methacrylate resin.

32. The separation media of claim 28, wherein said chromatographic matrix material is an NHS-activated agarose resin.

33. Separation media comprising the reaction product of:
(a) an amine-reactive chromatographic matrix material; and
(b) a polypeptide selected from the group consisting of:

| | |
|---|---|
| Ac-GDDHMCVYTTWGELIWCDNHEPGPEGGGK-NH_{2;} | (SEQ ID NO:144) |
| Ac-AGKYWCSFWGLQCKTGTPGPEGGGK-NH_{2;} | (SEQ ID NO:146) |
| Ac-AGPVDCKHHFWWCYWNGTPGPEGGGK-NH_{2;} | (SEQ ID NO:153) |
| Ac-GDDDHCYWFREWFNSECPHGEPGPEGGGK-NH_{2;} | (SEQ ID NO:154) |
| Ac-GDRRACSRDWSGALVWCAGHEPGPEGGGK-NH_{2;} | (SEQ ID NO:145) |
| Ac-AGYYWCNYWGLCPDQGTPGPEGGGK-NH_{2;} | (SEQ ID NO:155) |
| Ac-AGPHNCDDHYWYCKWFPGPEGGGK-NH_{2;} | (SEQ ID NO:150) |
| Ac-AGSYWCKIWDVCPQSPGPEGGGK-NH_{2;} | (SEQ ID NO:147) |
| Ac-AGKYWCNLWGVCPANPGPEGGGK-NH_{2;} | (SEQ ID NO:148) |
| Ac-AGAATCSTSYWYYQWFCTDSPGPEGGGK-NH_{2;} | (SEQ ID NO:151) |
| Ac-AGTYWCTFWELPCDPAPGPEGGGK-NH_{2;} | (SEQ ID NO:149) |
| Ac-AGYWYCWFPDRPECPLYPGPEGGGK-NH_{2;} | (SEQ ID NO:152) |
| Ac-GDSWVCWKAKWWEDKRCAPFGTPGPEGGGK-NH_{2;} | (SEQ ID NO:156) |
| Ac-GDNPMCWKKSWWEDAYCINHGTPGPEGGGK-NH_{2;} | (SEQ ID NO:157) |
| Ac-GDSWNCAFHHNEMVWCDDGGTPGPEGGGK-NH_{2;} | (SEQ ID NO:158) |
| Ac-GDWGECTVTSYGELIWCGGLEPGPEGGGK-NH_{2;} | (SEQ ID NO:159) |
| Ac-GDNPMCWRASWWEDAYCINHEPGPEGGGK-NH_{2;} | (SEQ ID NO:160) |
| Ac-GDNPMCWRAHWWEDAYCINHEPGPEGGGK-NH_{2;} | (SEQ ID NO:161) |
| Ac-GDDHMCVYTTWGELIWCDNHEPGPEG-X-NH_{2;} | (SEQ ID NO:162) |
| Ac-GDDHMCVYTTWGELPWCDNHEPG-X-Su-X-NH_{2;} | (SEQ ID NO:163) |
| Ac-GDDHMCVYTTWGELIWCDNHEPG-X-Z-X-NH_{2;} | (SEQ ID NO:164) |
| Ac-GDDHMCVYTTWGELIWCDNH-X-NH_{2;} | (SEQ ID NO:165) |
| Ac-GDDHMCVYTTWGELIWCDNH-X-Su-X-NH_{2;} | (SEQ ID NO:166) |
| Ac-GDDHMCVYTTWGELIWGDNH-X-Z-X-NH_{2;} | (SEQ ID NO:167) |
| Ac-DHMCVYTTWGELIWCDNHEPGPEGGGK-NH_{2;} | (SEQ ID NO:169) |
| Ac-EHMCVYTTWGELTWCDNHEPGPEGGGK-NH_{2;} | (SEQ ID NO:170) |
| Ac-ACVYTTWGELTWCDNHEPGPEGGGK-NH_{2;} | (SEQ ID NO:171) |
| Ac-TCVYTTWGELIWCDNHEPGPEGGGK-NH_{2;} | (SEQ ID NO:172) |
| Ac-ECVYTTWGELTWCDNHEPGPEGGGK-NH_{2;} | (SEQ ID NO:173) |
| Ac-VCVYTTWGELIWCDNHEPGPEGGGK-NH_{2;} | (SEQ ID NO:174) |
| Ac-[Nle]CVYTTWGELIWCDNHEPGPEGGGK-NH_{2;} | (SEQ ID NO:175) |
| Ac-CVYTTWGELIWCDNHEPGPEGGGK-NH_{2;} | (SEQ ID NO:176) |
| Ac-SRACSRDWSGALVWCAGHEPGPEGGGK-NH_{2;} | (SEQ ID NO:177) |
| Ac-RRACSRDWSGALVWCAGHEPGPEGGGK-NH_{2;} | (SEQ ID NO:178) |
| Ac-ERACSRDWSGALVWCAGHEPGPEGGGK-NH_{2;} | (SEQ ID NO:179) |
| Ac-ACSRDWSGALVWCAGHEPGPEGGGK-NH_{2;} | (SEQ ID NO:180) |
| Ac-TCSRDWSGALVWCAGHEPGPEGGGK-NH_{2;} | (SEQ ID NO:181) |
| Ac-ECSRDWSGALVWCAGHEPGPEGGGK-NH_{2;} | (SEQ ID NO:182) |
| Ac-VCSRDWSGALVWCAGHEPGPEGGGK-NH_{2;} | (SEQ ID NO:183) |
| Ac-GCSRDWSGALVWCAGHEPGPEGGGK-NH_{2;} | (SEQ ID NO:184) |
| and | |
| Ac-CSRDWSGALVWCAGHEPGPEGGGK-NH_{2,} | (SEQ ID NO:185) |

wherein: Ac-denotes N-terminal acetylation; —NH₂ denotes C-terminal amidation; [Nle] denotes norleucine; —X—NH₂ dentoes the C-terminal group —NH—(CH₂CH₂O)₂—CH₂CH₂—NH₂; —X—Su—X—NH₂ denotes the C-terminal group —NH—(CH₂CH₂O)₂—CH₂CH₂—NH—C:O—CH₂CH₂—C:O—NH—(CH₂CH₂O)₂—CH₂CH₂—NH₂; and —X—Z—X—NH₂ denotes the C-terminal group —NH—(CH₂CH₂O)₂—CH₂CH₂—NH—C:O—CH₂—O—(CH₂CH₂O)₂—CH₂—C:O—NH—(CH₂CH₂O)₂—CH₂CH₂13 NH₂.

34. The separation media of claim 33, wherein said matrix material is an aldehyde-functional methacrylate chromatographic resin.

35. The method of claim 33, wherein said matrix material is a formyl-substituted ethylene glycol-methacrylate copolymer support.

36. The method of claim 33, wherein said matrix material is an NHS-activated agarose support.

37. A method for separating a polypeptide target comprising at least one immunoglobulin Fc region amino acid sequence from a solution containing it comprising:
(a) contacting said solution with separation media as defined in claim 33 under binding conditions;
(b) removing unbound material; and
(c) eluting the bound polypeptide target from said separation media.

38. The method according to claim 37, wherein said polypeptide target can be an antibody or an antibody fragment containing all or part of an Fc region.

39. The method according to claim 37, wherein said polypeptide target is an antibody.

40. The method according to claim 39, wherein said antibody is selected from the group consisting of human IgG1, IgG2, IgG3, and IgG4.

41. A method for removing a polypeptide target comprising at least one immunoglobulin Fc region amino acid sequence from a solution containing it comprising:
(a) contacting said solution with separation media as defined in claim 33 under binding conditions; and
(b) removing unbound material.

42. The method according to claim 41, wherein said solution is selected from the group consisting of whole blood, plasma, transgenic milk, eggs of transgenic birds, and conditioned media.
